(19) United States
(12) Patent Application Publication
(10) Pub. No.: US 2026/0256908 A1
(43) Pub. Date: Sep. 3, 2026

(54) RNA Molecule

(71) Applicant: Imperial College Innovations Limited, South Kensington (GB)

(72) Inventors: Robin SHATTOCK, Paddington (GB); Karnyart SAMNUAN, Paddington (GB)

(73) Assignee: Imperial College Innovations Limited, South Kensington (GB)

(21) Appl. No.: 18/842,991

(22) PCT Filed: Mar. 3, 2023
(86) PCT No.: PCT/GB2023/050508

§ 371 Date: Aug. 30, 2024

(30) Foreign Application Priority Data
Mar. 4, 2022 (GB) 2203060.5

(51) Int. Cl. A61K 39/39; A61K 39/00; A61K 47/02; C12N 15/113; C12N 15/67

(52) U.S. Cl. CPC A61K 39/39; A61K 47/02; C12N 15/113; C12N 15/67; A61K 2039/53; C12N 2310/321; C12N 2310/335

(57) ABSTRACT

The invention relates to RNA molecules, to methods for preparing RNA molecules, and to methods for translating RNA molecules into protein. The invention extends to improved methods for forming RNA by in vitro transcription, and to the resultant RNA molecules. Furthermore, the invention relates to novel methods for enhancing the expression and/or translation of RNA, i.e. protein expression, and to methods for improving the stability of an RNA molecule. The invention also involves reducing the activation of innate sensing, interferon generation and/or degradation of an RNA molecule in a host. The invention also incorporates the use of the RNA molecules in vaccines and other therapeutic pharmaceutical compositions, and their use in immunisation and therapy, such as RNAi, gene therapy, gene editing and protein replacement.

The present invention relates to RNA molecules, and particularly, although not exclusively, to methods for preparing RNA molecules, and to methods for translating RNA molecules into protein. The invention extends to improved methods for forming RNA by in vitro transcription, and to the resultant RNA molecules. Furthermore, the invention relates to novel methods for enhancing the expression and/or translation of RNA, i.e. protein expression, and to methods for improving the stability of an RNA molecule. The invention also involves reducing the activation of innate sensing, interferon generation and/or degradation of an RNA molecule in a host. The invention also incorporates the use of the RNA molecules in vaccines and other therapeutic pharmaceutical compositions, and their use in immunisation and therapy, such as RNAi, gene therapy, gene editing and protein replacement.

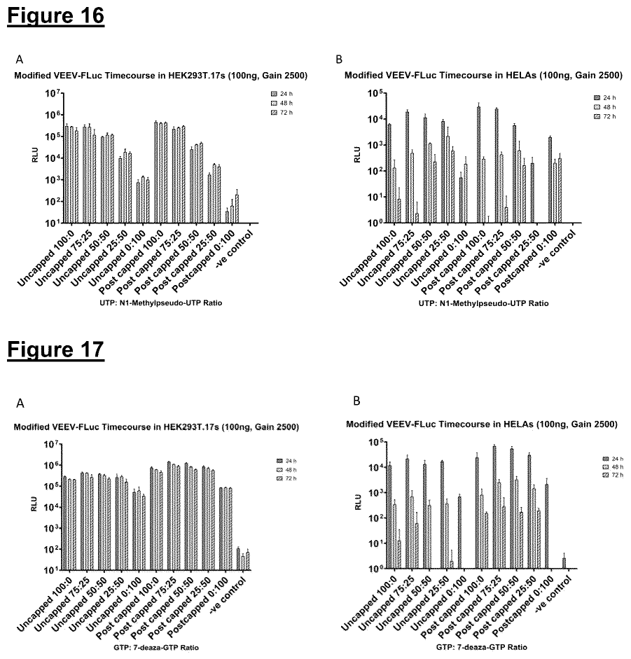

RNA vaccine and biotherapeutics have recently come of age, showing successful utility in the COVID-19 pandemic. The two lead COVID-19 candidates manufactured by Moderna and Pfizer both utilise the incorporation of N1-methyl-pseudouridine, a synthetic modified version of the nucleotide triphosphate (NTP) UTP [1]. This is used to reduce triggering of innate pathways that can restrict the expression of encoded proteins rendering the RNA ineffective with respect to therapeutic or vaccine indications [2,3]. The exact mechanism for improved expression is not fully understood and may be related to the suppression of highly immunogenic double stranded RNA (dsRNA) during in vitro transcription [11]. This approach, however, provides little or no advantage in the context of self-amplifying RNA (FIG. 16). Furthermore, the use of methyl-pseudouridine may not be optimal. Indeed, most cellular receptors detect most double-stranded RNA (dsRNA) structure by contacting the ribose backbone with minimal interaction with the bases. In this respect, ribose base modifications have been predicted to have beneficial effects, e.g. base 2′-methyl-NTPs in relation to innate recognition. However, efficient incorporation of 2′-methyl-NTPs in mRNA or saRNA in sequences over 100 bp has not previously been achievable. Furthermore, while previous studies have indicated that use of 2′-methyl-NTPs can reduce innate recognition, natural incorporation of 2′-methyl-NTPs in coding RNA is generally considered to inhibit or reduce translation [15].

Ribose 2′-OH replacement with a methyl group is a common, naturally occurring post-transcriptional modification, and 2′-O-methyl(2-OMe) modified RNAs can be less immunogenic and demonstrate greater stability. The 2-OMe modification consists of the methylation of the ribose 2′-OH moiety and can therefore occur in all four nucleotides and other non-canonical nucleotides. 2′-O-methyl modified RNA has been generated for short RNA sequences, such as aptamers (<20 bp in length), utilising in vitro transcription methods that maximise the synthesis of modified RNA from RNA polymerases such as T7, T3, SP6, KP34 and Syn5. However, bottlenecks presently exist for generating longer 2′-O-methyl modified RNA (>20 bp). For instance, strict transcription reaction mixture optimisations are required to support high and wide-ranging percentages of nucleotide substitution of wild-type (i.e. non-modified) NTPs. Furthermore, even the generation of 2′-O-methyl modified RNA aptamers by in vitro transcription is currently inefficient and can likely be improved. Overcoming these bottlenecks could improve the in vivo applications and potential of 2′-methyl RNAs intended for use as vaccines or biotherapeutics.

As discussed above, ribose 2′-O-methylation is a common type of RNA modification found in eukaryotic mRNA. Ribose 2′-O-methylation of the first and, sometimes, second cap-proximal nucleotides (N1 and N2, where N corresponds to any nucleotide), resulting in Cap1-(m7GpppNmN) or Cap2-(m7GpppNmNm) mRNA is known to serve as an important molecular signature of “self” to escape detection by the immune innate system [13]. While this process is common to higher eukaryotes, the role of numerous internal 2′-O-methylation sites, recently discovered within the human mRNA coding regions has been less well-characterised. Recent evidence has shown that the presence of a 2′-O-methylated mRNA codon decreases translation efficiency, especially when this modification is present at the second position of a codon [15]. This is further supported by data demonstrating that the presence of 2′-O-methylations in some mRNAs may selectively decrease translation elongation in vitro and in vivo [16, 17]. Thus, natural 2′-O-methylation within mRNA coding regions is understood to negatively regulate RNA translation and protein expression [18]. There is, therefore, a need to overcome the problems in the art, and provide an improved method for preparing a modified RNA molecule (for example, mRNA or self-amplifying RNA) which incorporates modified, non-natural nucleotide bases. There is also a need to provide improved methods for enhancing RNA translation and protein expression.

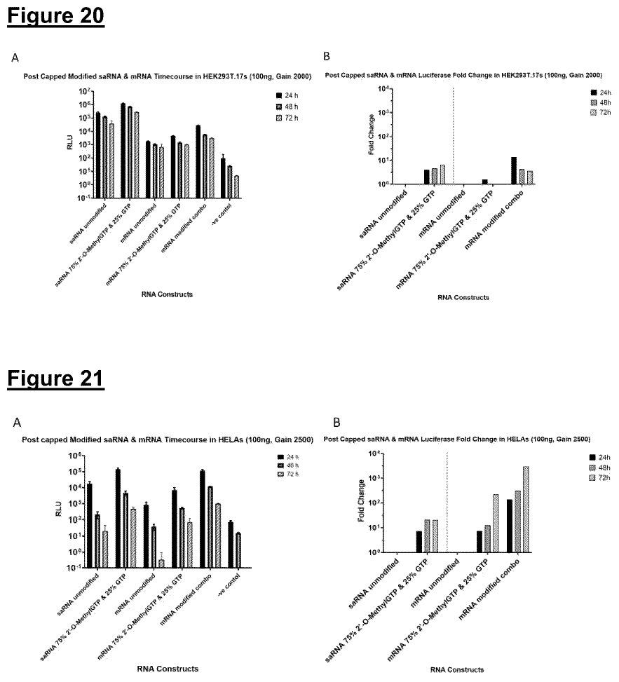

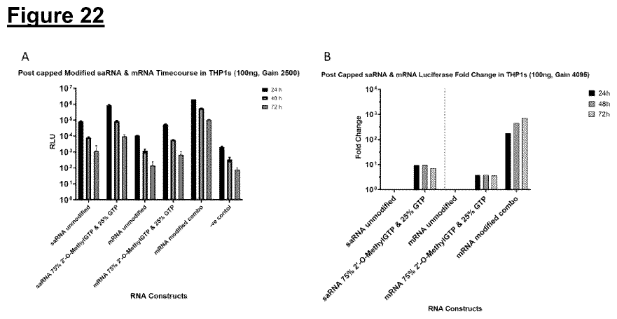

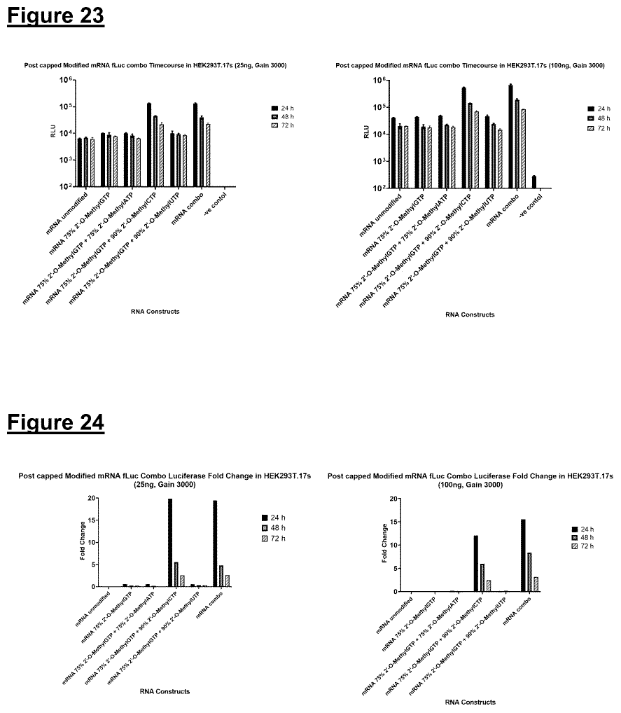

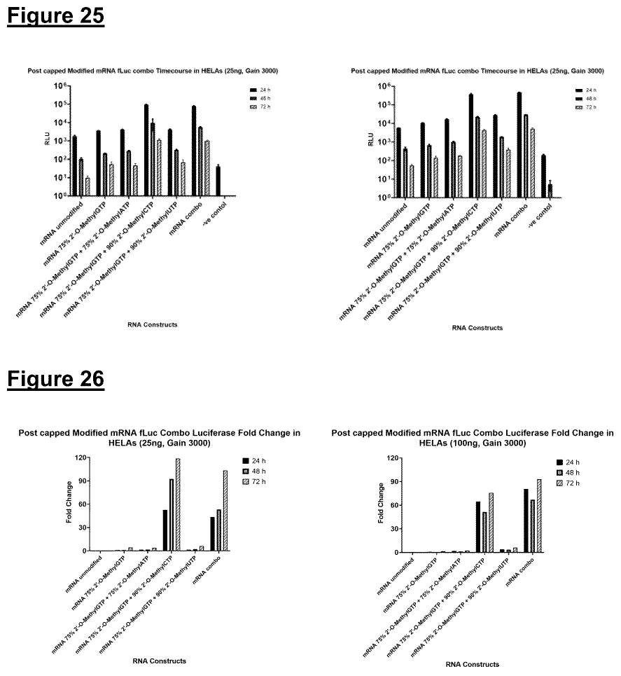

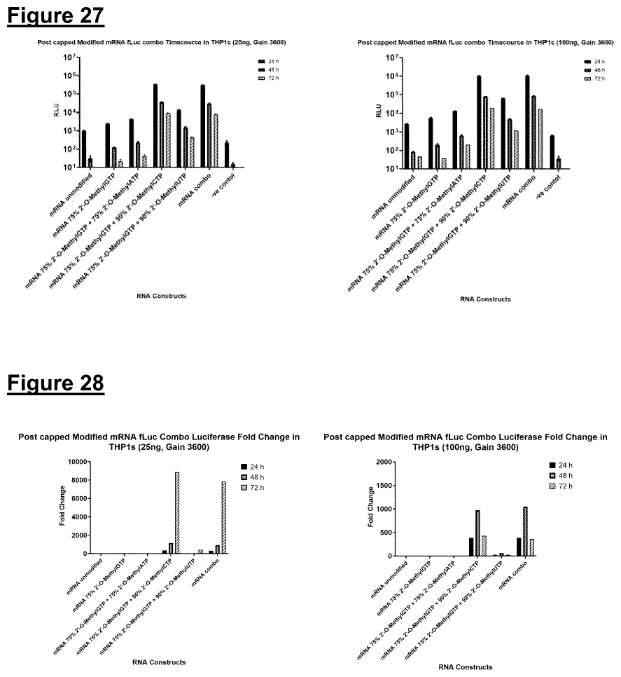

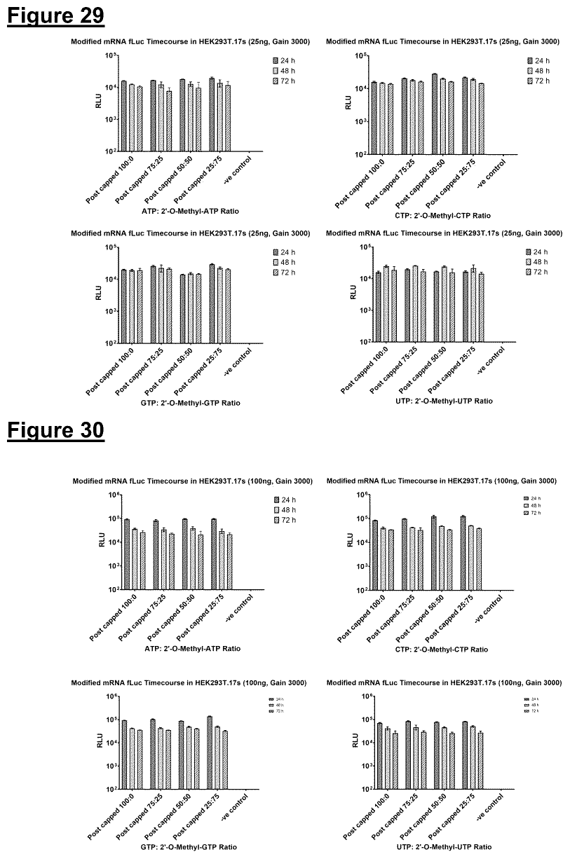

RNA is composed by the nucleotides ATP, CTP, GTP and UTP, however, a wide range of natural and synthetic modified nucleotides exist [4]. As described in the Examples, the inventors have demonstrated that incorporation of the modified base 2′-methyl-NTPs (2-OMe) surprisingly enhances the expression of RNA, both self-amplifying RNA (saRNA) and mRNA, in interferon-responsive cells. Substitution of ≥75% of GTP by 2′-methyl GTP (Gm) enhances saRNA expression 10-fold. Substitution of ≥75% of ATP, UTP or CTP by 2′-methyl modified nucleotides also enhanced saRNA expression in the cell types tested. The inventors believe that combined substitutions also can provide additional advantages. For example, the inventors have observed that substitution of ≥75% of CTP by 2′-methyl CTP (Cm) enhances mRNA expression (up to 10-fold; see FIGS. 33 and 34), while substitution of GTP, ATP, or UTP had little or no effect (see FIGS. 29-32). However, combined substitution of ≥75% or 2′methyl-GTP, and -CTP (ie Gm+Cm) or 2′-methyl-GTP,-ATP,-UTP and -CTP (i.e. Gm+Am+Cm+Um) surprisingly enhances mRNA expression by up to or greater than 100-fold (see FIGS. 23-28, reaching the levels of expression equivalent to saRNA (see FIGS. 21 and 22). This was totally unexpected given the evidence that, in the natural context, incorporation of 2′-O methylated NTP has been shown to reduce translation [16-18, 28-29]. Interestingly, these increases in translation were not observed when using modified nucleotides where the ribose 2′-OH was replaced by a 2′-fluoro group, suggesting that the effects are dependent upon the use of the bulkier alkyl group (e.g. the methyl group) as a replacement for the ribose 2′—OH group (see FIGS. 18 and 19). Furthermore, the level of translation enhancement was significantly greater than that seen with N1-methyl-pseudouridine for mRNA or saRNA in similar model systems (see FIG. 16).

The invention described herein, therefore, overcomes the prior art obstacles in generating RNA by substituting a significant proportion of one or more NTPs for 2′-methyl-NTPs and/or other modified NTPs that have not been amenable to efficient incorporation using conventional in vitro transcription (IVT) conditions.

Hence, in a first aspect of the invention, there is provided a method of preparing a modified RNA molecule, wherein the method comprises contacting, in the presence of at least 20 mM magnesium ions, (i) a template nucleic acid sequence, (ii) an RNA polymerase, and (iii) a plurality of nucleotide triphosphates (NTPs), one or more of which is a modified nucleotide triphosphate (NTP), wherein the RNA polymerase transcribes the template nucleic acid sequence to form an RNA molecule comprising at least 20 nucleotides, and wherein at least 25% of the constituent nucleotides in the RNA molecule are modified.

In a second aspect, there is provided use of 20 mM magnesium ions in a transcription reaction to prepare a modified RNA molecule comprising at least 20 nucleotides, wherein at least 25% of the constituent nucleotides in the RNA molecule are modified.

Advantageously, the inventors have devised novel and innovative in vitro transcription reaction conditions for use in the method of the invention. These reaction conditions include higher than previously used magnesium ions (i.e. >20 mM), and therefore enable significant levels of replacement of wild type (i.e. unmodified) nucleotides in the resultant RNA molecule. The use in the prior art of 2′-methyl-NTPs in short <20 bp RNA sequences (predominantly aptamers [5]) has been shown to prevent or reduce activation of innate immune responses through recognition by membrane and cytoplasmic pattern recognition receptors [6-8]. However, to date, it has not been possible to generate mRNA or saRNA sequences by in vitro transcription, of sizable length (>20 bp, 50 bp, or 100 bp, or more) where significant proportions (i.e. >25%) of single or multiple wild-type unmodified nucleotides are replaced by 2′-methyl-NTPs.

The in vitro transcription reaction mixture in the method of the invention requires the use of >20 mM magnesium ions and is demonstrably higher than some conventional in vitro transcription methods. 2′-methyl modified RNAs, both mRNA and saRNA, synthesised from the method exhibit significantly enhanced expression in interferon responsive cells. The inventors believe that the same reaction conditions may therefore facilitate the incorporation of other modified NTPs that have been refractory to existing IVT conditions.

Accordingly, the methods of the invention advantageously provide improvements to generating both mRNA and saRNA with significant 2′methyl-NTP substitution (>25% and even up to 100%), and demonstrate that such substitutions can significantly enhance expression of mRNA and saRNA in interferon-competent cells. This is highly likely to enhance the in vivo potential of RNA vaccines (for example, expressing microbial antigens for immunizing against infectious micro-organisms, such as viruses and bacteria etc., and also cancer vaccines expressing oncogenic antigens) and RNA biotherapeutics (for example, RNAi, protein replacement, gene editing and gene therapy etc.) whether delivered as mRNA or saRNA. Moreover, the same approach is also highly likely to facilitate the incorporation of other non-natural bases that have, to date, not been possible to incorporate into mRNA or saRNA [4].

Preferably, the method comprises the use of at least 30 mM magnesium ions, more preferably at least 35 mM magnesium ions, preferably at least 40 mM magnesium ions, and even more preferably at least 50 mM magnesium ions. Preferably, the method comprises the use of at least 60 mM magnesium ions, more preferably at least 70 mM magnesium ions, and even more preferably at least 75 mM magnesium ions. Preferably, the method comprises the use of at least 80 mM magnesium ions, more preferably at least 85 mM magnesium ions, more preferably at least 90 mM magnesium ions, and even more preferably at least 100 mM magnesium ions.

Preferably, the method comprises the use of between 50 mM and 100 mM magnesium ions, more preferably between 60 mM and 95 mM magnesium ions, more preferably between 65 mM and 90 mM magnesium ions, and even more preferably between 70 mM and 80 mM magnesium ions. Preferably, the method comprises the use of between 71 mM and 79 mM magnesium ions, more preferably between 73 mM and 77 mM magnesium ions.

The magnesium ions may be provided in the method as Mg²⁺ ions. The magnesium ions may be provided as magnesium acetate, magnesium citrate, magnesium sulphate, magnesium gluconate, magnesium lactate and so on. As described in the Examples, the inventors observed increased RNA expression when the magnesium ions were provided in the form of magnesium acetate but not magnesium chloride, and this was totally unexpected. Hence, preferably the magnesium ions are provided as magnesium acetate. Preferably, the magnesium ions are not provided as magnesium chloride.

The RNA may be single-stranded or double-stranded. The skilled person would appreciate that when the nucleic acid is double-stranded, for example double-stranded RNA, “bases in length” will refer to the length of base pairs. The RNA may be coding. For example, coding RNA may be used for therapeutic applications and vaccine applications.

The RNA may be non-coding. For example, non-coding RNA may be used for RNAi applications. The RNA may be selected from a group consisting of: messenger RNA (mRNA), micro RNA (miRNA); short interfering RNA (siRNA); short hairpin RNA (shRNA); anti-sense RNA; RNA aptamers; self-amplifying RNA (saRNA); interference RNA (RNAi); non-coding RNA; circular RNA; and small RNA [12].

Preferably, the RNA is self-amplifying RNA (saRNA) or messenger RNA (mRNA).

The RNA may be self-amplifying RNA (saRNA). The skilled person would appreciate that self-amplifying RNAs may contain the basic elements of mRNA (a cap, 5′ UTR, 3′UTR, an IRES, a viral polymerase, and poly(A) tail of variable length), but may be considerably longer (for example 9-12 kb).

The RNA may be messenger RNA (mRNA). mRNA may contain the basic elements of a cap, 5′ UTR, 3′UTR, IRES, and poly(A) tail of variable length.

The RNA molecule (which may be RNAi, saRNA or mRNA) may be at least 20, 21, 22 or 23 bases in length. The RNA molecule may be at least 24, 25, 26 or 27 bases in length. The RNA molecule may be at least 28, 29, 30, or 31 bases in length. The RNA molecule may be at least 32, 33, 34 or 35 bases in length. The RNA molecule may be at least 36, 37, 38 or 39 bases in length. The RNA molecule may be at least, at least 40, 41, 42, 43, 44 or 45 bases in length. The RNA molecule may be at least 46, 47, 48 or 49 bases in length.

The RNA molecule (which may be RNAi, saRNA or mRNA) may be at least at least 50 bases in length, at least 60 bases in length, at least 75 bases in length, at least 100 bases in length, at least 200 bases in length, at least 300 bases in length, at least 400 bases in length, at least 500 bases in length, at least 600 bases in length, at least 700 bases in length, at least 800 bases in length, or at least 900 bases in length.

The RNA molecule, most preferably saRNA or mRNA, may be at least 1000 bases in length, at least 2000 bases in length, at least 3000 bases in length, at least 4000 bases in length, at least 5000 bases in length, at least 6000 bases in length, at least 7000 bases in length, at least 8000 bases in length, at least 9000 bases in length, at least 10,000 bases in length, at least 11,000 bases in length or at least 12000 bases in length.

In one embodiment, RNA molecule is at least 6000 bases in length. In one embodiment, the RNA, most preferably saRNA or mRNA, is at least 6000 bases in length. In a preferred embodiment, the saRNA is at least 6000 bases in length.

The RNA, most preferably saRNA or mRNA, may be between 5000 and 20,000 bases in length, between 6000 and 15,000 bases in length, between 7000 and 14,000 bases in length, between 7500 and 13,000 bases in length, between 8000 and 12,000 bases in length, between 8500 and 11,000 bases in length, between 9000 and 10,000 bases in length.

The methods of the invention may be carried out in vivo, in vitro or ex vivo. However, most preferably the methods are carried out in vitro. Preferably, the methods comprise in vitro transcription (IVT).

Preferably, the method comprises the use of an RNA polymerase, which may be selected from a group consisting of: T7; T3; SP6; KP34; Syn5; or other DNA-dependent RNA polymerase; or a mutated variant of any of these RNA polymerases. Each of these RNA polymerases is able to maximise the synthesis of the modified RNA. Preferably, however, the method comprises the use of T7 RNA polymerase or a variant thereof. T7 RNA polymerase variants may include the following mutations (singly or in combination) associated with increased processivity and/or tolerance for incorporation of modified bases, specifically R425C, K631R, S633P, Y639F, Y639V, S641A, H784A, H784S, and/or H784G.

Preferably, the plurality of nucleotide triphosphates (NTPs) are selected from the group consisting of ATP, GTP, CTP and/or UTP. NTPs are the building blocks of RNA. Preferably, the method comprises the use of substantially equal proportions of each of ATP, GTP, CTP and/or UTP. However, in some embodiments, the method may comprise different ratios between each of ATP, GTP, CTP and/or UTP, examples of which are given below. This will depend on the sequence of the template nucleic acid and resultant RNA molecule to be transcribed therefrom.

Preferably, the method comprises the use of the plurality of nucleotide triphosphates at a concentration of at least 1 mM, 2 mM, 3 mM or 4 mM. Preferably, the method comprises the use of the plurality of nucleotide triphosphates at a concentration of at least 5 mM, 6 mM or 7 mM. More preferably, the method comprises the use of the plurality of nucleotide triphosphates at a concentration of at least 8 mM, 9 mM, 10 mM. Suitably, the method comprises the use of the plurality of nucleotide triphosphates at a concentration of greater than 10 mM. For example, the method may comprise the use of the plurality of nucleotide triphosphates at a concentration of at least 12 mM, 14 mM, or 16 mM. The method may also comprise the use of the plurality of nucleotide triphosphates at a concentration of at least 18 mM, 20 mM, or 22 mM. In other embodiments, the method may comprise the use of the plurality of nucleotide triphosphates at a concentration of at least 25 mM, 50 mM, 75 mM or 100 mM.

The one or more modified NTP used in the method may be selected from a group consisting of: a modified adenosine-5′-triphosphate (ATP); a modified cytidine-5′-triphosphate (CTP); a modified guanosine-5′-triphosphate (GTP), a modified uridine-5′-triphosphate (UTP) and/or a modified thymidine-5′-triphosphate (TTP).

Preferably, the method comprises using one or more modified NTP which comprises a modified ATP, a modified CTP, a modified GTP, a modified UTP, and/or a modified TTP. Preferably, the method comprises using at least one modified NTP selected from a group consisting of: a modified ATP, a modified CTP, a modified GTP, a modified UTP, and/or a modified TTP. Preferably, the method comprises using at least two modified NTPs selected from a group consisting of: a modified ATP, a modified CTP, a modified GTP, a modified UTP and/or a modified TTP. Preferably, the method comprises using at least three modified NTPs selected from a group consisting of: a modified ATP, a modified CTP, a modified GTP, a modified UTP, and/or a modified TTP. Preferably, the method comprises using at least four modified NTPs selected from a group consisting of: a modified ATP, a modified CTP, a modified GTP, a modified UTP and/or a modified TTP. Preferably, the method comprises using at least five modified NTPs selected from a group consisting of: a modified ATP, a modified CTP, a modified GTP, a modified UTP, and/or a modified TTP.

In one embodiment, the one or more modified NTP may comprise a 2′-substituted group in which the OH group normally at the 2′ position can be replaced with a halogen, an optionally substituted aromatic group, a NH₂, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.

Alternatively, or additionally, the one or more modified NTP may comprise a substituted nucleobase. The nucleobase may be substituted with a halogen, an optionally substituted aromatic group, a NH₂, a N₃, an OH, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl. Alternatively, or additionally, an oxo group in the nucleobase may be replaced by a=S group.

It may be appreciated, when discussing the optional substituents and the size of the alkyl, alkenyl and/or alkynyl, this applies to both the optionally substituted alkyl, alkenyl and alkynyl groups per se as well as the optionally substituted O-alkyl, O-alkenyl and O-alkynyl groups.

The alkyl, alkenyl or alkynyl may be an optionally substituted C₁-C₂₀ alkyl, alkenyl or alkynyl NTP, wherein the alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl. Preferably, the alkyl, alkenyl or alkynyl may be an optionally substituted C₁-C₁₀ alkyl, alkenyl or alkynyl NTP, wherein the alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.

In some embodiments, the alkyl, alkenyl or alkynyl may be an optionally substituted C₁₋₆ alkyl, a C₂₋₆ alkenyl or a C₂₋₆ alkynyl, wherein the, or each alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl. In some embodiments, the alkyl, alkenyl or alkynyl may be an optionally substituted C₁₋₃ alkyl, a C₂₋₃ alkenyl or a C₂₋₃ alkynyl, wherein the, or each alkyl, alkenyl or alkynyl is substituted with one or more substituents selected from the group consisting of an oxo, OH, OMe, NH₂ and NRH, wherein R is C₁₋₆ alkyl, C₂₋₆ alkenyl or C₂₋₆ alkynyl. More preferably, the alkyl, alkenyl or alkynyl may be an optionally substituted C₁₋₃ alkyl, a C₂₋₃ alkenyl or a C₂₋₃ alkynyl, wherein the, or each alkyl, alkenyl or alkynyl is unsubstituted.

Accordingly, in some embodiments, the alkyl, alkenyl or alkynyl may be methyl, ethyl, propyl, ethenyl, propenyl, ethynyl or propynyl, optionally substituted with one or more substituents selected from the group consisting of an oxo, OH, OMe, NH₂ and NRH, wherein R is C₁₋₆ alkyl, C₂₋₆ alkenyl or C₂₋₆ alkynyl. Accordingly, the alkyl, alkenyl or alkynyl may be methyl, hydromethyl, acetyl, formyl, carbamoylmethyl, methoxycarbonylmethyl, carboxyhydroxymethyl, carboxymethylaminomethyl, oxyacetic acid methyl ester or isopentenylaminomethyl.

The halogen may be a chlorine, or bromine. The halogen may be fluorine. Therefore, the one or more modified NTP may comprise a 2′-substituted fluoro NTP.

The optionally substituted aromatic group may be a C₆-C₁₂ optionally substituted aromatic group. The optionally substituted aromatic group may be optionally substituted phenyl.

Preferably, the one or more modified NTP comprises a 2′-methyl modified NTP.

Most preferably, the one or more modified NTP comprises a 2′-O-methyl modified NTP.

There are at least 28 identified natural 2′-O-methyl modifications (Nm) to ribonucleosides. Accordingly, the one or more modified NTP may be selected from a group consisting of: 2′-O-methyladenosine (Am); 1,2′-dimethyl-adenosine (m¹Am); N6,2′-O-dimethyl-adenosine (m⁶Am); N⁶,N⁶,2-O-trimethyl-adenosine (m^{6,6}Am); 2′-O-methylinosine (Im); 1,2′-O-dimethylinosine (m¹Im); 2′-O-methylguanosine (Gm); 1,2′-O-dimethylguanosine (m¹Gm); N2,2′-O-dimethylguanosine (m²Gm); N2,N2, 2′-O-trimethylguanosine (m^{2,2}Gm); N2,7,2′-O-trimethyl-guanosine (m^{2,7}Gm); 2′-O-methylcytidine; N⁴,2′-O-dimethyl cytidine (m⁴Cm); N⁴,N⁴,2-O-trimethyl-cytidine (m^{4,4} Cm); 5,2′-O-dimethyl-cytidine (m⁵Cm); N⁴-acetyl-2′-O-methyl-cytidine (ac⁴Cm); 2′-O-methyl-5-hydromethyl-cytidine (hm⁵Cm); 5-formyl-2′-O-methyl-cytidine (f⁵Cm); 2′-O-methyluridine (Um); 3,2′-O-dimethyluridine (m³Um); 5,2′-O-dimethyluridine (m⁵Um or Tm); 2-thio-2′-O-methyl-uridine (s²Um); 2′-O-methyl-pseudouridine (Ym); 5-carbamoylmethyl-2′-O-methyluridine (ncm⁵Um); 5-methoxycarbonylmethyl-2′-O-methyluridine (mcm⁵Um); 5-(carboxyhydroxymethyl)-2′-O-methyluridine methyl ester (mchm⁵Um); 5-carboxymethylaminomethyl-2′-O-methyluridine (cmbm⁵Um); 2′-O-methyluridine 5-oxyacetic acid methyl ester (mcmo⁵Um); 5-(isopentenylaminomethyl)-3′O-methyluridine (inm⁵Um); and any other synthetic NTP that contains a 2′-O-methylated ribose.

Preferably, therefore, the one or more modified NTP comprises a 2′-O-methyl modified ATP (2′-O-methyl-ATP; also known as “2-OMe ATP” or “Am”), a 2′-O-methyl modified CTP (2′-O-methyl-CTP; also known as “2-OMe CTP” or “Cm”), a 2′-O-methyl modified GTP (2′-O-methyl-GTP; also known as “2-OMe GTP” or “Gm”), a 2′-O-methyl modified UTP (2′-O-methyl-UTP; also known as “2-OMe UTP” or “Um”), and/or a 2′-O-methyl modified TTP (2′-O-methyl-TTP; also known as “2-OMe TTP” or “Tm”).

Preferably, the method comprises using one or more modified NTP which comprises a 2′-O-methyl-ATP, a 2′-O-methyl-CTP, a 2′-O-methyl-GTP, a 2′-O-methyl-UTP, and/or 2′-O-methyl-TTP. Preferably, the method comprises using at least one modified NTP selected from a group consisting of: 2′-O-methyl-ATP, a 2′-O-methyl-CTP, a 2′-O-methyl-GTP, a 2′-O-methyl-UTP, and 2′-O-methyl-TTP. Preferably, the method comprises using at least two modified NTPs selected from a group consisting of: 2′-O-methyl-ATP, a 2′-O-methyl-CTP, a 2′-O-methyl-GTP, a 2′-O-methyl-UTP, and 2′-O-methyl-TTP. Preferably, the method comprises using at least three modified NTPs selected from a group consisting of: 2′-O-methyl-ATP, a 2′-O-methyl-CTP, a 2′-O-methyl-GTP, a 2′-O-methyl-UTP, and 2′-O-methyl-TTP. Preferably, the method comprises using at least four modified NTPs selected from a group consisting of: 2′-O-methyl-ATP, a 2′-O-methyl-CTP, a 2′-O-methyl-GTP, a 2′-O-methyl-UTP, and 2′-O-methyl-TTP. Preferably, the method comprises using at least five modified NTPs selected from a group consisting of: 2′-O-methyl-ATP, a 2′-O-methyl-CTP, a 2′-O-methyl-GTP, a 2′-O-methyl-UTP, and 2′-O-methyl-TTP.

Most preferably, however, the method comprises using at least four modified NTPs selected from a group consisting of: 2′-O-methyl-ATP, a 2′-O-methyl-CTP, a 2′-O-methyl-GTP, and a 2′-O-methyl-UTP.

The inventors attempted to achieve maximum incorporation of the modified base(s) in the modified RNA molecule. For GTP and ATP, about 75% replacement has been achieved, and for UTP and CTP about 90% replacement has been achieved. The inventors have observed that, for saRNA, most preferentially a modified GTP is incorporated into the modified RNA molecule and, for mRNA, most preferentially modified GTP and modified CTP are incorporated.

Thus, in embodiments in which the modified RNA molecule is saRNA, preferably the RNA molecule comprises modified 2′-O-methyl-GTP, and unmodified ATP, CTP and/or UTP. Furthermore, in embodiments in which the modified RNA molecule is mRNA, preferably the RNA molecule comprises modified 2′-O-methyl-GTP and modified 2′-O-methyl CTP, and unmodified ATP, and/or UTP.

In one embodiment, the one or more modified NTP may comprise 2′-O-methyl ATP, represented herein as formula [I]:

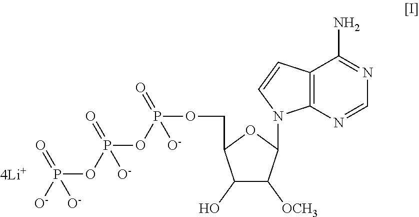 [SMILES: COC1C(O)C(COP(=O)([O-])OP(=O)([O-])OP(=O)([O-])[O-])OC1n1ccc2c(N)ncnc21]

In another embodiment, the one or more modified NTP may comprise 2′-O-methyl CTP, represented herein as formula [II]:

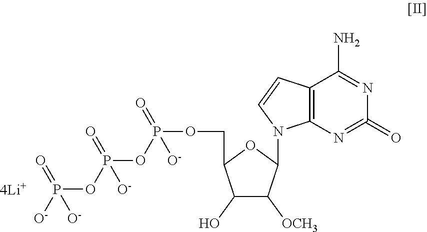

In another embodiment, the one or more modified NTP may comprise 2′-O-methyl GTP, represented herein as formula [III]:

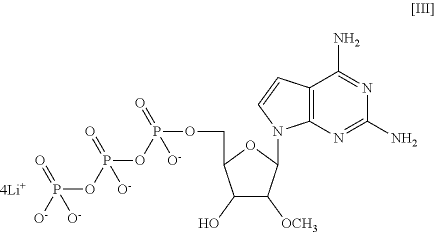 [SMILES: COC1C(O)C(COP(=O)([O-])OP(=O)([O-])OP(=O)([O-])[O-])OC1n1ccc2c(N)nc(N)nc21]

In another embodiment, the one or more modified NTP may comprise 2′-O-methyl UTP, represented herein as formula [IV]:

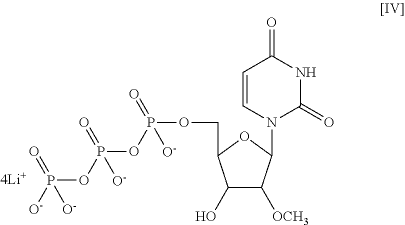 [SMILES: COC1C(O)C(COP(=O)([O-])OP(=O)([O-])OP(=O)([O-])[O-])OC1n1ccc(=O)[nH]c1=O]

As described in the Examples, the inventors observed surprisingly increased saRNA expression levels when the one or more modified NTP comprises modified GTP. Thus, preferably, the one or more modified NTP comprises modified GTP, more preferably 2′-O-methyl modified GTP. Furthermore, for mRNA expression levels when the one or more modified NTP comprises modified CTP, preferably a combination of CTP plus GTP. Thus, preferably for mRNA, the one or more modified NTP comprises modified CTP plus GTP, more preferably 2′-O-methyl modified CTP plus 2′-O-methyl modified GTP.

Preferably, one or more of the NTPs used in the invention are modified, in that they are not naturally occurring. Preferably, at least 30%, 35% or 40% of the constituent nucleotides in the resultant RNA molecule are modified, wherein the modified nucleotides may comprise adenine, cytosine, guanine and/or uracil. More preferably, at least 45%, 50% or 55% of the constituent nucleotides in the resultant RNA molecule are modified, wherein the modified nucleotides may comprise adenine, cytosine, guanine and/or uracil. Even more preferably, at least 60%, 65% or 70% of the constituent nucleotides in the RNA molecule are modified, wherein the modified nucleotides may comprise adenine, cytosine, guanine and/or uracil. Still more preferably, at least 75%, 80% or 85% of the constituent nucleotides in the RNA molecule are modified, wherein the modified nucleotides may comprise adenine, cytosine, guanine and/or uracil. Most preferably, at least 90%, 95%, 96%, 97%, 98%, 99% or 100% of the constituent nucleotides in the RNA molecule are modified, wherein the modified nucleotides may comprise adenine, cytosine, guanine and/or uracil.

As described in the examples, in an embodiment in which the RNA molecule being produced is mRNA, the inventors surprisingly observed that substituting ≥75% of CTP enhanced expression by up to 10-fold. Furthermore, combined substitution of ≥75% of wild-type nucleotides with 2′-methyl-CTP plus-GTP, or -GTP, -ATP, -CTP and/or -UTP enhanced mRNA expression by up to 100-fold, similar to levels of expression demonstrated by saRNA with 75% Gm substitution. This same level of enhancement was also surprisingly observed when ≥75% of wild-type nucleotides were replaced with just 2′-O-methyl-GTP and 2′-O-methyl-CTP, suggesting that replacement of both GTP and CTP provides the dominant effect.

Furthermore, as described in the examples, in an embodiment in which the RNA molecule being produced by the method is saRNA, the inventors found that substituting ≥75% of GTP with 2′-methyl GTP (Gm) in saRNA led to surprisingly enhanced expression. In addition, ≥75% Gm modifications showed the largest effect, enhancing expression by 10-fold or more in different cell lines. Substitution with 2′-O-methyl-ATP, UTP or CTP improved expression of saRNA in difference cell types although to a lesser extent than GTP. Further increases in expression are also achieved with combined substitutions Gm plus Cm or combinations of Gm, Cm, Am and Um. For example, preferably the method comprises replacing different proportions of the different bases in the modified RNA molecule.

As described herein, for mRNA, the inventors have been able to substitute all four bases at about 75% substitutions. However, the more complex architecture of saRNA means that it is much more challenging to achieve such high rates of modified bases compared to those seen with mRNA.

Accordingly, preferably the method comprises using a combination of two, three or four different modified NTPs selected from a group consisting of: 2′-O-methyl-ATP, a 2′-O-methyl-CTP, a 2′-O-methyl-GTP, and a 2′-O-methyl-UTP. The ratio between the two, three or four different modified NTPs may be varied.

Accordingly, the inventors have assessed different combinations of modified bases, as follows:—

For example, for saRNA, combinations of modified bases may be:—
1. 75% Gm;
2, 75% Gm plus 90% Am and 90% Um;
3. 75% Gm plus 75% Cm (with or without 90% Am+Um);
4. 75% Gm plus 50% Cm (with or without 90% Am+Um); or
5. 50% Gm plus 50% Cm (with or without 90% Am+Um).

Preferably, the method comprises using a combination of four different modified NTPs selected from a group consisting of: 2′-O-methyl-ATP, a 2′-O-methyl-CTP, a 2′-O-methyl-GTP, and a 2′-O-methyl-UTP. The ratio between the four different modified NTPs may be varied.

Alternatively, for saRNA, combinations of modified bases may be:—
1. 50% Gm+30% Cm+50% Am+30% Um;
2. 50% Gm+90% Cm+50% Am+30% Um;
3. 50% Gm+30% Cm+50% Am+90% Um;
4. 50% Gm+90% Cm+50% Am+90% Um;
5. 25% Gm+30% Cm+25% Am+60% Um;
6. 25% Gm+90% Cm+50% Am+60% Um;
7. 75% Gm+30% Cm+50% Am+60% Um;
8. 75% Gm+60% Cm+50% Am+75% Um;
9. 75% Gm+90% Cm+50% Am+60% Um;
10. 50% Gm+30% Cm+25% Am+60% Um;
11. 50% Gm+90% Cm+25% Am+60% Um;
12. 50% Gm+30% Cm+75% Am+60% Um;
13. 50% Gm+90% Cm+75% Am+60% Um;
14. 25% Gm+60% Cm+50% Am+30% Um;
15. 25% Gm+60% Cm+50% Am+90% Um;
16. 75% Gm+60% Cm+50% Am+30% Um;
17. 75% Gm+60% Cm+50% Am+90% Um;
18. 50% Gm+60% Cm+25% Am+30% Um;
19. 50% Gm+60% Cm+25% Am+90% Um;
20. 50% Gm+60% Cm+75% Am+30% Um;
21. 50% Gm+60% Cm+75% Am+90% Um;
22. 25% Gm+60% Cm+25% Am+60% Um;
23. 75% Gm+60% Cm+25% Am+60% Um;
24. 25% Gm+60% Cm+75% Am+60% Um;
25. 75% Gm+60% Cm+75% Am+60% Um;
26. 50% Gm+60% Cm+50% Am+60% Um;
27. 50% Gm+60% Cm+75% Am+75% Um;
28. 50% Gm+60% Cm+60% Am+60% Um; or
29. 50% Gm+50% Cm+50% Am+50% Um; or any other percentage ratio.

For mRNA, combinations of modified bases may be:—
1. 75% Gm+90% Cm (with or without 90% Am+Um);
2. 75% Gm+75% Cm (with or without 90% Am+Um);
3. 50% Gm+50% Cm (with or without 90% Am+Um); or
4. 25% Gm+25% Cm (with or without 90% Am+Um).

Alternatively, for mRNA, combinations of modified bases may be:—
1. 50% Gm+30% Cm+50% Am+30% Um;
2. 50% Gm+90% Cm+50% Am+30% Um;
3. 50% Gm+30% Cm+50% Am+90% Um;
4. 50% Gm+90% Cm+50% Am+90% Um;
5. 25% Gm+30% Cm+25% Am+60% Um;
6. 25% Gm+90% Cm+50% Am+60% Um;
7. 75% Gm+30% Cm+50% Am+60% Um;
8. 75% Gm+60% Cm+50% Am+75% Um;
9. 75% Gm+90% Cm+50% Am+60% Um;
10. 50% Gm+30% Cm+25% Am+60% Um;
11. 50% Gm+90% Cm+25% Am+60% Um;
12. 50% Gm+30% Cm+75% Am+60% Um;
13. 50% Gm+90% Cm+75% Am+60% Um;
14. 25% Gm+60% Cm+50% Am+30% Um;
15. 25% Gm+60% Cm+50% Am+90% Um;
16. 75% Gm+60% Cm+50% Am+30% Um;
17. 75% Gm+60% Cm+50% Am+90% Um;
18. 50% Gm+60% Cm+25% Am+30% Um;
19. 50% Gm+60% Cm+25% Am+90% Um;
20. 50% Gm+60% Cm+75% Am+30% Um;
21. 50% Gm+60% Cm+75% Am+90% Um;
22. 25% Gm+60% Cm+25% Am+60% Um;
23. 75% Gm+60% Cm+25% Am+60% Um;
24. 25% Gm+60% Cm+75% Am+60% Um;
25. 75% Gm+60% Cm+75% Am+60% Um;
26. 50% Gm+60% Cm+50% Am+60% Um;
27. 50% Gm+60% Cm+75% Am+75% Um;
28. 50% Gm+60% Cm+60% Am+60% Um; or
29. 50% Gm+50% Cm+50% Am+50% Um; or any other percentage ratio.

In another embodiment, the one or more modified NTP may comprise 7-deazaguanosine-5-triphosphate (7-deaza-GTP), which may be represented herein as formula [V]:

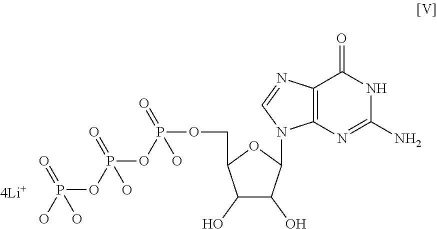 [SMILES: Nc1nc2c(ncn2C2OC(COP(=O)(O)OP(=O)(O)OP(=O)(O)O)C(O)C2O)c(=O)[nH]1]

In another embodiment, the one or more modified NTP may comprise 7-deazaadenosine-5-triphosphate (7-deaza-ATP), which may be represented herein as formula [VI]:

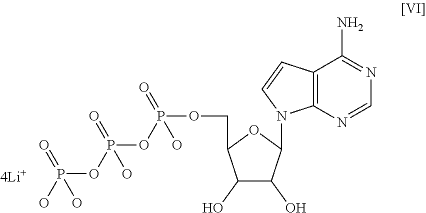 [SMILES: Nc1ncnc2c1ccn2C1OC(COP(=O)(O)OP(=O)(O)OP(=O)(O)O)C(O)C1O]

In another embodiment, the one or more modified NTP may comprise N1-methyl-pseudouridine, which may be represented herein as formula [VII]:

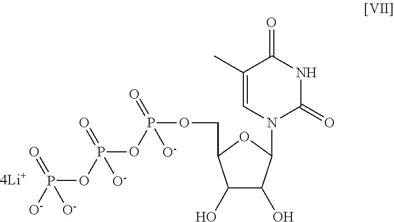 [SMILES: Cc1cn(C2OC(COP(=O)([O-])OP(=O)([O-])OP(=O)([O-])[O-])C(O)C2O)c(=O)[nH]c1=O]

In some embodiments, the method may comprise combining one or more 2-O-methyl modified NTP, as defined herein, with one or more other modified NTP bases, which may not be a 2-O-methyl modified NTP.

By way of example, in an embodiment in which the RNA molecule is mRNA, in which one preferred combination may be Gm+Cm, UTP may additionally be replaced with N′ methyl-pseudouridine or fluorinated UTP, and so on. Similarly, in an embodiment in which the RNA molecule is saRNA, in which one preferred modified NTP is Gm, UTP may additionally be replaced with N′ methyl-pseudouridine or fluorinated UTP.

Thus, the method may comprise combining:
(i) one or more modified NTP comprising a 2′-substituted group in which the OH group normally at the 2′ position is replaced with a halogen, an optionally substituted aromatic group, a NH₂, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl; and
(ii) one or more modified NTP, which is not a 2′-O-methyl modified NTP.

The halogen may be a chlorine, fluorine or bromine. The halogen may be fluorine.

Preferably, the method comprises combining:
(i) one or more 2-O-methyl modified NTP selected from a group consisting of a 2′-O-methyl modified ATP (2′-O-methyl-ATP): a 2′-O-methyl modified CTP (2′-O-methyl-CTP); a 2′-O-methyl modified GTP (2′-O-methyl-GTP); and a 2′-O-methyl modified UTP (2′-O-methyl-UTP); and
(ii) one or more other modified NTP bases, which is not a 2-O-methyl modified NTP.

For example, the one or more other modified NTP bases, which is not a 2-O-methyl modified NTP, may be N′ methyl-pseudouridine, fluorinated UTP, 2′-chloro NTP, 2′-ethyl, 2′-bromo NTP, 2′-amino NTP, 2′-fluoro NTP, or 2′-deoxy NTP, etc. Preferably, therefore, the one or more other modified NTP bases, which is not a 2-O-methyl modified NTP, is an alternative 2′-modified nucleotide.

For example, the 2′-chloro NTP may be 2′-chloro ATP, 2′-chloro CTP, 2′-chloro GTP, 2′-chloro TTP and/or 2′-chloro UTP.

For example, the 2′-ethyl NTP may be 2′-ethyl ATP, 2′-ethyl CTP, 2′-ethyl GTP, 2′-ethyl TTP and/or 2′-ethyl UTP.

For example, the 2′-bromo NTP may be 2′-bromo ATP, 2′-bromo CTP, 2′-bromo GTP, 2′-bromo TTP and/or 2′-bromo UTP.

For example, the 2′-amino NTP may be 2′-amino ATP, 2′-amino CTP, 2′-amino GTP, 2′-amino TTP and/or 2′-amino UTP.

For example, the 2′-fluoro NTP may be 2′-fluoro ATP, 2′-fluoro CTP, 2′-fluoro GTP, 2′-fluoro TTP and/or 2′-fluoro UTP.

The 2′-deoxy NTP may be 2′-deoxy ATP, 2′-deoxy CTP, 2′-deoxy GTP, 2′-deoxy TTP and/or 2′-deoxy UTP.

Preferably, the method comprises combining one or more 2-O-methyl modified NTP with one or more alternative 2′-modified nucleotide, which may be selected from the alternative 2′-modified nucleotides listed in Table 7 below.

TABLE 7
Combinations of 2-O-methyl modified NTP with
an alternative 2′-modified nucleotide

| Modified | |
|---|---|
| Nucleotides | Modified Nucleotide Combination |
| Gm | 2′-deoxy GTP |
| Gm | 2′-Fluoro GTP |
| Cm | 2′-deoxy CTP |
| Cm | 2′-Fluoro CTP |
| Am | 2′-deoxy ATP |
| Am | 2′-Fluoro ATP |
| Um | 2′-deoxy UTP |
| Um | 2′-Fluoro UTP |
| Tm | 2′-deoxy TTP |
| Tm | 2′-Fluoro TTP |
| Gm + Cm | 2′-deoxy GTP + 2′-deoxy CTP |
| Gm + Cm | 2′-Fluoro GTP + 2′-Fluro CTP |
| Am + Um | 2′-deoxy ATP + 2′-deoxy UTP |
| Am + Um | 2′-Fluoro ATP + 2′-Fluoro UTP |
| Am + Tm | 2′-deoxy ATP + 2′-deoxy TTP |
| Am + Tm | 2′-Fluoro ATP + 2′-Fluoro TTP |
| Gm + Am | 2′-deoxy GTP + 2′-deoxy ATP |
| Gm + Am | 2′-Fluoro GTP + 2′-Fluoro ATP |
| Gm + Um | 2′-deoxy GTP + 2′-deoxy UTP |
| Gm + Um | 2′-Fluoro GTP + 2′-Fluoro UTP |
| Gm + Tm | 2′-deoxy GTP + 2′-deoxy TTP |
| Gm + Tm | 2′-Fluoro GTP + 2′-Fluoro TTP |
| Cm + Am | 2′-deoxy CTP + 2′-deoxy ATP |
| Cm + Am | 2′-Fluoro CTP + 2′-Fluoro ATP |
| Cm + Um | 2′-deoxy CTP + 2′-deoxy UTP |
| Cm + Um | 2′-Fluoro CTP + 2′-Fluoro UTP |
| Cm + Tm | 2′-deoxy CTP + 2′-deoxy TTP |
| Cm + Tm | 2′-Fluoro CTP + 2′-Fluoro TTP |
| Gm + Cm + Am | 2′-deoxy GTP + 2′-deoxy CTP + 2′- |
| | deoxy ATP |
| Gm + Cm + Am | 2′-Fluoro GTP + 2′-Fluoro CTP + |
| | 2′-Fluoro ATP |
| Gm + Cm + Um | 2′-deoxy GTP + 2′-deoxy CTP + 2′- |
| | deoxy UTP |
| Gm + Cm + Um | 2′-Fluoro GTP + 2′-Fluoro CTP + 2′- |
| | Fluoro UTP |
| Gm + Cm + Tm | 2′-deoxy GTP + 2′-deoxy CTP + 2′- |
| | deoxy TTP |
| Gm + Cm + Tm | 2′-Fluoro GTP + 2′-Fluoro CTP + 2′- |
| | Fluoro TTP |
| Gm + Cm + Am + | 2′-deoxy GTP + 2′-deoxy CTP + 2′- |
| Um | deoxy ATP + 2′-deoxy UTP |
| Gm + Cm + Am + | 2′-Fluoro GTP + 2′-Fluoro CTP + 2′- |
| Um | Fluoro ATP + 2′Fluoro UTP |
| Gm + Cm + Am + | 2′-deoxy GTP + 2′-deoxy CTP + 2′- |
| Tm | deoxy ATP + 2′-deoxy UTP |
| Gm + Cm + Am + | 2′-Fluoro GTP + 2′-Fluoro CTP + 2′- |
| Tm | Fluoro ATP + 2′Fluoro UTP |
| Um | Pseudouridine or N1-methyl pseudouridine |
| Am + Um | Pseudouridine or N1-methyl pseudouridine |
| Gm + Cm + Um | Pseudouridine or N1-methyl pseudouridine |
| Gm + Cm + Am + | Pseudouridine or N1-methyl pseudouridine |
| Um | |
| Cm | 5-Methylcytosine |
| Gm + Cm | 5-Methylcytosine |
| Gm + Cm + Am + | 5-Methylcytosine |
| Um | |
| Gm + Cm + Am + | 5-Methylcytosine |
| Tm | |

Such combinations can be used in any ratio to prepare the nucleic acids or modified RNA of the invention. For example, about 0.1%, 1%, 5%, 10%, 15%, 20%, 25%, 30%, 35%, 40%, 45%, 50%, 60%, 65%, 70%, 75%, 80%, 85%, 90%, 95%, 99% or 99.9% of the one or more 2-O-methyl modified NTP may be combined with about 99.9%, 99%, 95%, 90%, 85%, 80%, 75%, 70%, 65%, 60%, 55%, 50%, 45%, 40%, 35%, 30%, 25%, 20%, 15%, 10%, 5%, 1% or 0.1%, respectively, of the one or more other modified NTP bases, which is not a 2-O-methyl modified NTP.

Unless otherwise noted, the modified nucleotides may be completely substituted for natural nucleotides of nucleic acid or modified RNA of the invention. As a non-limiting example, the natural nucleotide guanidine, cytosine, adenine, thymine or uracil may be partially substituted (e.g., about 1%, 5%, 10%, 15%, 20%, 25%, 30%, 35%, 40%, 45%, 50%, 60%, 65%, 70%, 75%, 80%, 85%, 90%, 95% or 99.9%) with at least one of the modified nucleotides disclosed herein.

As a non-limiting example, 2-O-methyl modified NTP may be partially substituted (e.g., about 1%, 5%, 10%, 15%, 20%, 25%, 30%, 35%, 40%, 45%, 50%, 55% 60%, 65%, 70%, 75%, 80%, 85%, 90%, 95% or 99.9%) with at least one alternative 2′-modified nucleotide (e.g. 2′-deoxy GTP, 2′-Fluoro GTP). As a non-limiting example, 2-O-methyl modified GTP (Gm) may be partially substituted (e.g., about 1%, 5%, 10%, 15%, 20%, 25%, 30%, 35%, 40%, 45%, 50%, 55% 60%, 65%, 70%, 75%, 80%, 85%, 90%, 95% or 99.9%) with at least one alternative 2′ modified nucleotide (e.g. 2′-deoxy GTP, 2′-Fluoro GTP).

Accordingly, as an example, if Gm is being used at 90%, it could be used in combination with 2′-deoxy GTP (dGTP) with the following ratios (89:1%, 85:5%, 80:10%, 75:15%, 70:20%, 76:25%, 60:30%, 55:35%, 50:40%, 45:45%, 40:50%, 35:55% 30:60%, 25:65%, 20:70%, 15:75%, 10:80%, 5:85%).

Also, additional 2′-modified NTPs, including 2′-chloro, 2′-ethyl, 2′-bromo, 2′-amino etc. may be used.

The method may further comprise use of DTT. This is thought to enhance RNA yield and RNA polymerase activity.

The method may further comprise use of a pH buffer, such as HEPES, though the skilled person would appreciate that other buffers are available.

The method may further comprise the use of a crowding agent, for example Poly (ethylene glycol) (PEG) of various molecular weights. For example, the crowding agent may be PEG200 or any additional crowding agent [32].

The method may further comprise use of an RNase inhibitor. This prevents RNA degradation if any RNase is present in the reaction.

The method may further comprise use of spermidine. This is thought to improve the activity of the RNA polymerase and therefore the RNA yield.

The template nucleic acid sequence is preferably transcribed by the RNA polymerase to produce the modified RNA molecule. Preferably, therefore, the template nucleic acid sequence comprises DNA. The template nucleic acid may be made synthetically, for example doggybone DNA, or by PCR, rolling circle amplification, or synthetic amplification. The template nucleic acid may comprise a vector, and is preferably a plasmid.

In one embodiment, the template nucleic acid may be transcribed to create the resultant modified RNA molecule, which can be described as being a biotherapeutic RNA molecule, i.e. one which is used therapeutically to treat, prevent or ameliorate a disease in a patient. The template nucleic acid sequence may encode a therapeutic protein, which is derived from an animal or a human, and which treats, prevents or ameliorates disease in a subject, preferably a human or animal subject. For example, therapeutic uses of the encoded therapeutic protein may include protein replacement, gene editing (e.g. CRISPR-Cas9), gene therapy, or RNAi, and so on.

The biotherapeutic RNA molecule may be selected from a group consisting of: messenger RNA (mRNA), micro RNA (miRNA); short interfering RNA (siRNA); short hairpin RNA (shRNA); anti-sense RNA; RNA aptamer; self-amplifying RNA (saRNA); interference RNA (RNAi); non-coding RNA; circular RNA; and small RNA.

In one embodiment, the template nucleic acid may encode a protein and peptide derived from a plant. Preferably, the protein and peptide is a plant antigen. The plant antigen may be derived from Ricinus communis.

In another embodiment, the template nucleic acid encodes an antigen, in which case the resultant modified RNA molecule can be described as being a vaccine, i.e. one which is used prophylactically to prevent a disease in a patient, or one which is used therapeutically to ameliorate disease, such as cancer. For example, the template nucleic acid sequence may encode an antigen which is derived from a virus, a bacteria, a mycoplasma, a fungus, an animal, a plant, an alga, a parasite, or a protozoan, or other organism which causes a disease in a subject, preferably a human or animal Preferably, the template nucleic acid sequence is from a virus or a bacterium. For example, the template nucleic acid may encode at least a portion of a viral spike protein.

In another embodiment, the template nucleic acid may encode a tumour immunogen or antigen, or cancer immunogen or antigen. The tumour immunogen and antigen may be a peptide-containing tumour antigen, such as a polypeptide tumour antigen or glycoprotein tumour antigens.

The tumour antigen may be (a) a full length molecule associated with cancer cells, (b) a homolog and modified form of the same, including molecules with deleted, added and/or substituted portions, and (c) a fragment of the same.

Suitable tumour immunogens include: class I-restricted antigens recognized by CD 8+ lymphocytes or class II-restricted antigens recognized by CD4+ lymphocytes.

The tumour antigen may be an antigen that is associated with a cancer selected from the group consisting of: a testis cancer, melanoma, lung cancer, head and neck cancer, NSCLC, breast cancer, gastrointestinal cancer, bladder cancer, colorectal cancer, pancreatic cancer, lymphoma, leukaemia, renal cancer, hepatoma, ovarian cancer, gastric cancer and prostate cancer.

The tumour antigen may be selected from:
(a) cancer-testis antigens such as NY-ESO-I, SSX2, SCPI as well as RAGE, BAGE, GAGE and MAGE family polypeptides, for example, GAGE-I, GAGE-2, MAGE-I, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours);
(b) mutated antigens, for example, p53 (associated with various solid tumours, e.g., colorectal, lung, head and neck cancer), p21/Ras (associated with, e.g., melanoma, pancreatic cancer and colorectal cancer), CDK4 (associated with, e.g., melanoma), MUMI (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukaemia), triosephosphate isomerase, KIA 0205, CDC-27, and LDLR-FUT;
(c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukaemia), WT 1 (associated with, e.g., various leukaemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-I (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer);
(d) shared antigens, for example, melanoma-melanocyte differentiation antigens such as MART-1/Melan A, gplOO, MCIR, melanocyte-stimulating hormone receptor, tyrosinase, tyrosinase related protein-1/TRPI and tyrosinase related protein-2/TRP2 (associated with, e.g., melanoma);
(e) prostate-associated antigens, such as PAP, PSA, PSMA, PSH-PI, PSM-Pl, PSM-P2, associated with e.g., prostate cancer; and/or
(f) immunoglobulin idiotypes (associated with myeloma and B cell lymphomas, for example).

The template nucleic acid may encode a eukaryotic polypeptide. In one embodiment the eukaryotic polypeptide is a mammalian polypeptide. The mammalian polypeptide may be selected from the group consisting of: an enzyme; an enzyme inhibitor; a hormone; an immune system protein; a receptor; a binding protein; a transcription or translation factor; tumour growth suppressing protein; a structural protein and a blood protein.

The enzyme may be selected from the group consisting of: chymosin; gastric lipase; tissue plasminogen activator; streptokinase; a cholesterol biosynthetic or degradative steriodogenic enzyme; kinases; phosphodiesterases; methylases; de-methylases; dehydrogenases; cellulases; proteases; lipases; phospholipases; aromatases; cytochromes; adenylate or guanylaste cyclases and neuramidases.

The enzyme inhibitor may be tissue inhibitor of metalloproteinase (TIMP). The hormone may be growth hormone.

The immune system protein may be selected from the group consisting of: a cytokine; a chemokine; a lymphokine; erythropoietin; an integrin; addressin; selectin; homing receptors; T cell receptors and immunoglobulins.

The cytokine may be an interleukin, for example IL-2, IL-4 and/or IL-6, colony stimulating factor (CSF), granulocyte colony stimulating factor (G-CSF), granulocyte-macrophage colony stimulating factor (GM-CSF) or tumour necrosis factor (TNF).

The chemokine may be a macrophage inflammatory protein-2 and/or a plasminogen activator.

The lymphokine may be an interferon.

The immunoglobulin may be a natural, modified or chimeric immunoglobulin or a fragment thereof. Preferably, the immunoglobulin is a chimeric immunoglobulin having dual activity such as antibody enzyme or antibody-toxin chimera.

The hormone may be selected from the group consisting of: insulin, thyroid hormone, catecholamines, gonadotrophines, trophic hormones, prolactin, oxytocin, dopamine, bovine somatotropin, leptins; growth hormones (e.g., human grown hormone), growth factors (e.g., epidermal growth factor, nerve growth factor, insulin-like growth factor and the like).

The receptor may be a steroid hormone receptor or a peptide receptor. Preferably, the receptor is a growth factor receptor.

The binding protein may be a growth factor binding protein.

The tumour growth suppressing protein may be a protein that inhibits angiogenesis.

The structural protein may be selected from the group consisting of: collagen; fibroin; fibrinogen; elastin; tubulin; actin; and myosin.

The blood protein may be selected from the group consisting of thrombin; serum albumin; Factor VII; Factor VIII; insulin; Factor IX; Factor X; tissue plasminogen activator; protein C; von Wilebrand factor; antithrombin III; glucocerebrosidase; erythropoietin granulocyte colony stimulating factor (GCSF) or modified Factor VIII; and anticoagulants.

In one preferred embodiment, the template nucleic acid may encode a cytokine which is capable of regulating lymphoid homeostasis, preferably a cytokine which is involved in and preferably induces or enhances development, priming, expansion, differentiation and/or survival of T cells. Thus, preferably, the cytokine is an interleukin. Most preferably, IL-2, IL-7, IL-12, IL-15, or IL-21.

The template nucleic acid may encode a protein that is capable of enhancing reprogramming of somatic cells to cells having stem cell characteristics.

The protein that is capable of enhancing reprogramming of somatic cells to cells having stem cell characteristics may be selected from the group consisting of: OCT4, SOX2, NANOG, LIN28, P53, ART-4, BAGE, ss-catenin/m, Bcr-abL CAMEL, CAP-1, CASP-8, CDC27/m, CD 4/m, CEA, CLAUDIN-12, c-MYC, CT, Cyp-B, DAM, ELF2M, ETV6-AML1, G250, GAGE, GnT-V, GaplOO, HAGE, HER-2/neu, HPV-E7, HPV-E6, HAST-2, hTERT (or hTRT), LAGE, LDLR/FUT, MAGE-A, MAGE-B, MAGE-C, MART-1/Melan-A, MC1R, Myosin/m, MUC1, MUM-1, -2, -3, NA88-A, NF1, NY-ESO-1, NY-BR-1, pl90 minor BCR-abL, Plac-1, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT, preferably WT-1.

Preferably, MAGE-A is selected from the group consisting of: MAGE-A 1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A 12

Preferably, the protein that is capable of enhancing reprogramming of somatic cells to cells having stem cell characteristics is OCT4, SOX2, LF4; c-MYC; NANOG; LIN28.

The template nucleic acid may encode a biomolecule that is utilised for the modification of cells ex vivo for cell-therapy indications. Thus, preferably the template nucleic acid may encode a protein selected from the group consisting of an immunoglobulin, a T-cell receptor and NK receptor.

The template nucleic acid may encode a protein that is capable of regulating expression of endogenous host genes, for example an interfering RNA, such as small RNAs, siRNA or microRNAs.

In an embodiment, the template nucleic acid may encode an innate inhibitor protein, as described in WO2020/254804, PCT/GB2021/053362 or PCT/GB2021/053361, which counteracts the innate immune response in a subject administered with a vaccine comprising the resultant RNA molecule.

In accordance with a third aspect, there is provided an RNA molecule obtained or obtainable by the method according to the first aspect.

In a fourth aspect, there is provided a pharmaceutical composition comprising the RNA molecule according to the third aspect and a pharmaceutically acceptable vehicle.

In a fifth aspect, there is provided a method of preparing the pharmaceutical composition according to the fourth aspect, the method comprising contacting the RNA molecule according to the third aspect and a pharmaceutically acceptable vehicle.

In a sixth aspect, there is provided the RNA molecule according to the third aspect, or the pharmaceutical composition according to the fourth aspect, for use as a medicament.

In a seventh aspect, there is provided the RNA molecule according to the third aspect, or the pharmaceutical composition according to the fourth aspect, for use in treating, preventing or ameliorating a disease in a subject, such as cancer.

In an eighth aspect, there is provided a method of treating, preventing or ameliorating a disease in a subject, such as cancer, the method comprising administering, or having administered, to a subject in need thereof, a therapeutically effective amount of the RNA molecule according to the third aspect, or the pharmaceutical composition according to the fourth aspect.

In a ninth aspect, there is provided a vaccine composition comprising the RNA molecule according to the third aspect, or the pharmaceutical composition according to the fourth aspect.

The vaccine may comprise a suitable adjuvant. Examples of adjuvants may include an aluminium salt, a synthetic form of DNA, a carbohydrate, a tablet binder, an ion exchange resin, a preservative, a polymer, an emulsion and/or a lipid. Examples of adjuvants may include monosodium glutamate, sucrose, dextrose, aluminum bovine, human serum albumin, cytosine phosphoguanine, potassium phosphate, plasdone C, anhydrous lactose, cellulose, polacrilin potassium, glycerine, asparagine, citric acid, potassium phosphate magnesium sulfate, iron ammonium citrate, 2-phenoxyethanol, aluminium, beta-propiolactone, bovine extract, DOPC, EDTA, formaldehyde, Cytosine phosphoguanine (CpG), QS21, saponin, Monophosphoryl lipid A (MPLA), squalene, thimerosal, phenol, potassium aluminum sulfate, potassium glutamate, sodium borate, sodium metabisulphite, urea, PLGA, PVA, PLA, PVP, cyclodextrin-based stabilisers, oil in water emulsion adjuvants and/or lipid-based adjuvants.

In a tenth aspect, there is provided the RNA molecule according to the third aspect, the pharmaceutical composition according to the fourth aspect or the vaccine according to the ninth aspect, for use in stimulating an immune response in a subject.

The immune response may be stimulated against a protozoa, bacterium, virus, fungus or cancer. Thus, the vaccine may be used to express a microbial or pathogenic antigen for immunizing against infectious micro-organisms, such as viruses and bacteria etc. However, the vaccine may also be used to express an oncogenic antigen, for preventing cancer.

In an eleventh aspect of the invention, there is provided a method of vaccinating a subject, the method comprising administering, or having administered, to a subject in need thereof, a therapeutically effective amount of the RNA molecule according to the third aspect, the pharmaceutical composition according to the fourth aspect or the vaccine according to the ninth aspect.

It will be appreciated that RNA molecules can have significant utility in a wide range of therapeutic applications, for example RNAi, inhibitory RNA, RNA aptamers, and so on, in which translation of the RNA molecule does not necessarily need to occur. However, applications in which the RNA molecule does need to be translated include various vaccine and biotherapeutic approaches, such as the use of mRNA, saRNA, circular RNA, or any RNA sequence that is able to undergo translation and generate a peptide or protein in vivo.

For example, the peptide or protein that is encoded by the RNA molecule may be involved in protein replacement, gene editing and gene therapy etc.

It will also be appreciated that there is a growing trend for the use, in particular, of mRNA and saRNA vaccines, in which the RNA molecule encodes a pathogenic such as a viral coat protein or region thereof, or an oncogenic antigen, and it is this antigen which is expressed in an immunised host organism and which triggers an immune response to the pathogen or the tumour.

As described in the examples, not only have the inventors shown that the method of the first aspect can be used to effectively prepare modified RNA molecules comprising modified NTPs, they have also surprisingly demonstrated significant elevations in RNA translation (i.e. protein expression), particularly when the modified RNA molecule is either mRNA or saRNA. For example, substituting ≥75% of CTP in mRNA enhanced expression by up to 10-fold, and a combined substitution of ≥75% of wild-type nucleotides with 2′-methyl-CTP and -GTP, or -GTP-ATP,-CTP and/or -UTP enhanced mRNA expression by up to 100-fold, which are similar to levels of elevated expression also demonstrated by saRNA with 75% Gm substitution. This same level of enhanced expression was also surprisingly observed in mRNA when ≥75% of wild-type nucleotides were replaced with just 2′-O-methyl-GTP and 2′-O-methyl-CTP.

As described in the Examples, rather than directly measuring the amount of luciferase protein produced, the inventors have instead measured the enhancement of expression in terms of luciferase activity. Luciferase activity can, therefore, be considered as a surrogate of RNA translation, because it should directly relate to the amount of translation. Moreover, the luciferase data described in the examples can be used a surrogate of a therapeutic, essentially mirroring the expression of a therapeutic protein. However, as described herein, when the term “10-fold” or the like is used, it means that there is in fact a 10-fold increase in luciferase activity. It will be appreciated that the increase in expression or RNA translation may be related to the RNA being more stable, and so more protein is made before it is degraded. Accordingly, there may not be a change in translation efficiency (i.e. ribosomal speed), but an increase in the overall time that ribosomes have for making more protein.

In summary, these data, when taken together, demonstrate that modified NTPs, in particular 2′-O-methyl modified bases, can be used to increase the level of RNA translation for protein production in vitro and in vivo, the latter being especially advantageous when the RNA molecule is used as a vaccine or a therapeutic biomolecule. Based on the unexpected findings that incorporation of 2′-O modified bases enhances, rather than reduces, expression as seen with natural incorporation of 2′-O methylated bases in coding RNA, the inventors believe that their data also support the finding that modified bases can be used to enhance the stability of RNA, as well as reduce the activation of innate sensing, interferon generation and/or degradation of an RNA molecule. These effects of 2′-O modified bases on RNA are particularly unexpected in view of the current understanding in the art that natural incorporation of 2-O-methyl bases actually inhibits RNA translation, i.e. protein production.

Accordingly, the inventors believe that any 2′-OH substituted modified NTPs can be used to:
(i) enhance the expression and/or translation of an RNA molecule (in particular, mRNA or saRNA, and ideally using 2′-O-methyl NTPs);
(ii) enhance the stability of an RNA molecule (any type of RNA irrespective of its length and with any 2′-OH substitution described herein); and/or
(iii) reduce activation of innate sensing, interferon generation and/or degradation of an RNA molecule (any type of RNA irrespective of its length and with any 2′-OH substitution described herein).

Thus, in a twelfth aspect of the invention, there is provided one or more modified nucleotide triphosphate (NTP), for use in:
(i) enhancing the expression and/or translation of an RNA molecule comprising the one or more modified NTP;
(ii) enhancing the stability of an RNA molecule comprising the one or more modified NTP; and/or
(iii) reducing the activation of innate sensing, interferon generation and/or degradation of an RNA molecule comprising the one or more modified NTP,
wherein the one or more modified NTP comprises a 2′-substituted group in which the OH group normally at the 2′ position is replaced with a halogen, an optionally substituted aromatic group, a NH₂, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.

Also, in a thirteenth aspect, there is provided a method of:
(i) enhancing the expression and/or translation of an RNA molecule comprising one or more modified NTP;
(ii) enhancing the stability of an RNA molecule comprising one or more modified NTP; and/or
(iii) reducing the activation of innate sensing, interferon generation and/or degradation of an RNA molecule comprising one or more modified NTP,
wherein the method comprises incorporating one or more modified nucleotide triphosphate (NTP) into the RNA molecule, and
wherein the one or more modified NTP comprises a 2′-substituted group in which the OH group normally at the 2′ position is replaced with a halogen, an optionally substituted aromatic group, a NH₂, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl, and
thereby enhancing the expression and/or translation of the RNA molecule comprising the one or more modified NTP, enhancing the stability of the RNA molecule comprising the one or more modified NTP, and/or reducing the activation of innate sensing, interferon generation and/or degradation of the RNA molecule comprising the one or more modified NTP.

Enhancing expression of translation of a modified RNA molecule is an important aspect of the invention.

Accordingly, in a fourteenth aspect, there is provided a method of enhancing the translation of an RNA molecule, the method comprising translating an RNA molecule comprising one or more 2′-O-methyl modified nucleotide triphosphate (NTP), wherein the level of translation in the presence of the one or more 2′-O-methyl modified NTP is greater than the level of translation in the absence of the one or more 2′-O-methyl modified NTP.

In a fifteenth aspect, there is provided the use of one or more 2′-O-methyl modified nucleotide triphosphate (NTP) in an RNA molecule, for enhancing the translation of the RNA molecule.

In a sixteenth aspect, there is provided one or more 2′-O-methyl modified nucleotide triphosphate (NTP) in an RNA molecule, for use in enhancing the translation of the RNA molecule.

The methods or uses may be carried out in vivo, in vitro or ex vivo. However, most preferably the methods are carried out in vivo.

In some embodiments, the one or more modified NTP may be incorporated into the RNA molecule using the method of the first aspect. Thus, the method may comprise contacting, in the presence of at least 20 mM magnesium ions, (i) a template nucleic acid sequence, (ii) an RNA polymerase, and (iii) a plurality of nucleotide triphosphates (NTPs), one or more of which is a modified nucleotide triphosphate (NTP), wherein the RNA polymerase transcribes the template nucleic acid sequence to form the RNA molecule.

The one or more modified NTP may be as defined in relation to the first aspect of the invention. Preferably, the one or more modified NTP comprises a 2′-methyl modified NTP. Most preferably, the one or more modified NTP comprises a 2′-O-methyl modified NTP. Accordingly, the one or more modified NTP may be selected from a group consisting of: 2′-O-methyladenosine (Am); 1,2′-dimethyl-adenosine (m¹Am); N6,2′-O-dimethyl-adenosine (m⁶Am); N⁶,N⁶,2-O-trimethyl-adenosine (m^{6,6}Am); 2′-O-methylinosine (Im); 1,2′-O-dimethylinosine (m¹Im); 2′-O-methylguanosine (Gm); 1,2′-O-dimethylguanosine (m¹Gm); N2,2′-O-dimethylguanosine (m²Gm); N2,N2, 2′-O-trimethylguanosine (m^{2,2}Gm); N2,7,2′-O-trimethyl-guanosine (m^{2,7}Gm); 2′-O-methylcytidine; N⁴,2′-O-dimethyl cytidine (m⁴Cm); N⁴,N⁴,2-O-trimethyl-cytidine (m^{4,4} Cm); 5,2′-O-dimethyl-cytidine (m⁵Cm); N⁴-acetyl-2′-O-methyl-cytidine (ac⁴Cm); 2′-O-methyl-5-hydromethyl-cytidine (hm⁵Cm); 5-formyl-2′-O-methyl-cytidine (f⁵Cm); 2′-O-methyluridine (Um); 3,2′-O-dimethyluridine (m³Um); 5,2′-O-dimethyluridine (m⁵Um or Tm); 2-thio-2′-O-methyl-uridine (s²Um); 2′-O-methyl-pseudouridine (Ym); 5-carbamoylmethyl-2′-O-methyluridine (ncm⁵Um); 5-methoxycarbonylmethyl-2′-O-methyluridine (mcm⁵Um); 5-(carboxyhydroxymethyl)-2′-O-methyluridine methyl ester (mchm⁵Um); 5-carboxymethylaminomethyl-2′-O-methyluridine (cmbm⁵Um); 2′-O-methyluridine 5-oxyacetic acid methyl ester (mcmo⁵Um); 5-(isopentenylaminomethyl)-3′O-methyluridine (inm⁵Um); and any other synthetic NTP that contains a 2′-O-methylated ribose.

Preferably, therefore, the one or more modified NTP comprises a 2′-O-methyl modified ATP, a 2′-O-methyl modified CTP, a 2′-O-methyl modified GTP, a 2′-O-methyl modified UTP, and/or a 2′-O-methyl modified TTP.

Preferably, the method comprises using one or more modified NTP which comprises a 2′-O-methyl-ATP, a 2′-O-methyl-CTP, a 2′-O-methyl-GTP, a 2′-O-methyl-UTP and/or a 2′-O-methyl modified TTP. Preferably, the method comprises using at least one modified NTP selected from a group consisting of: 2′-O-methyl-ATP, a 2′-O-methyl-CTP, a 2′-O-methyl-GTP, a 2′-O-methyl-UTP, and a 2′-O-methyl modified TTP. Preferably, the method comprises using at least two modified NTPs selected from a group consisting of: 2′-O-methyl-ATP, a 2′-O-methyl-CTP, a 2′-O-methyl-GTP, a 2′-O-methyl-UTP, and a 2′-O-methyl modified TTP. Preferably, the method comprises using at least three modified NTPs selected from a group consisting of: 2′-O-methyl-ATP, a 2′-O-methyl-CTP, a 2′-O-methyl-GTP, a 2′-O-methyl-UTP, and a 2′-O-methyl modified TTP. Preferably, the method comprises using at least four modified NTPs selected from a group consisting of: 2′-O-methyl-ATP, a 2′-O-methyl-CTP, a 2′-O-methyl-GTP, a 2′-O-methyl-UTP, and a 2′-O-methyl modified TTP. Preferably, the method comprises using at least five modified NTPs selected from a group consisting of: 2′-O-methyl-ATP, a 2′-O-methyl-CTP, a 2′-O-methyl-GTP, a 2′-O-methyl-UTP, and a 2′-O-methyl modified TTP. Preferably, however, the method comprises using at least four modified NTPs selected from a group consisting of: 2′-O-methyl-ATP, a 2′-O-methyl-CTP, a 2′-O-methyl-GTP, and a 2′-O-methyl-UTP.

Preferably, the one or more modified NTP comprises 2′-O-methyl modified GTP. In embodiments in which the modified RNA molecule is saRNA, preferably the RNA molecule comprises modified 2′-O-methyl-GTP, and unmodified ATP, CTP and/or UTP. Furthermore, in embodiments in which the modified RNA molecule is mRNA, preferably the RNA molecule comprises modified 2′-O-methyl-GTP and modified 2′-O-methyl CTP, and unmodified ATP, and/or UTP.

The type of RNA molecule may be as described in relation to the first aspect. Thus, the RNA may be single-stranded or double-stranded. The RNA may be coding. For example, coding RNA may be used for therapeutic applications and vaccine applications. The RNA may be non-coding. For example, non-coding RNA may be used for RNAi applications. The RNA may be selected from a group consisting of: messenger RNA (mRNA); self-amplifying RNA (saRNA); micro RNA (miRNA); short interfering RNA (siRNA); short hairpin RNA (shRNA); anti-sense RNA; RNA aptamers; interference RNA (RNAi); non-coding RNA; circular RNA; and small RNA.

Advantageously, incorporation of a modified NTP in a non-translated RNA molecule (e.g. miRNA, siRNA, shRNA, anti-sense RNA, RNA aptamer, RNAi, non-coding RNA, circular RNA or small RNA) increases its stability and reduces its detection by innate immune receptors.

Preferably, the RNA is self-amplifying RNA (saRNA) or messenger RNA (mRNA).

The length of the RNA molecule may be as described in relation to the first aspect.

The RNA molecule (which may be RNAi, saRNA or mRNA) may be at least 20, 21, 22 or 23 bases in length. The RNA molecule may be at least 24, 25, 26 or 27 bases in length. The RNA molecule may be at least 28, 29, 30, or 31 bases in length. The RNA molecule may be at least 32, 33, 34 or 35 bases in length. The RNA molecule may be at least 36, 37, 38 or 39 bases in length. The RNA molecule may be at least, at least 40, 41, 42, 43, 44 or 45 bases in length. The RNA molecule may be at least 46, 47, 48 or 49 bases in length.

The RNA molecule (which may be RNAi, saRNA or mRNA) may be at least at least 50 bases in length, at least 60 bases in length, at least 75 bases in length, at least 100 bases in length, at least 200 bases in length, at least 300 bases in length, at least 400 bases in length, at least 500 bases in length, at least 600 bases in length, at least 700 bases in length, at least 800 bases in length, or at least 900 bases in length.

For example, the RNA molecule (which may be saRNA or mRNA), may be at least 1000 bases in length, at least 2000 bases in length, at least 3000 bases in length, at least 4000 bases in length, at least 5000 bases in length, at least 6000 bases in length, at least 7000 bases in length, at least 8000 bases in length, at least 9000 bases in length, at least 10,000 bases in length, at least 11,000 bases in length or at least 12000 bases in length. In one embodiment, RNA molecule is at least 6000 bases in length. In one embodiment, the RNA, most preferably saRNA or mRNA, is at least 6000 bases in length. In a preferred embodiment, the saRNA is at least 6000 bases in length. The RNA, most preferably saRNA or mRNA, may be between 5000 and 20,000 bases in length, between 6000 and 15,000 bases in length, between 7000 and 14,000 bases in length, between 7500 and 13,000 bases in length, between 8000 and 12,000 bases in length, between 8500 and 11,000 bases in length, between 9000 and 10,000 bases in length.

Preferably, one or more of the NTPs used in the methods or uses are modified, in that they are not naturally occurring. Preferably, at least 30%, 35% or 40% of the constituent nucleotides in the RNA molecule are modified, wherein the modified nucleotides may comprise adenine, cytosine, guanine and/or uracil. More preferably, at least 45%, 50% or 55% of the constituent nucleotides in the RNA molecule are modified, wherein the modified nucleotides may comprise adenine, cytosine, guanine and/or uracil. Even more preferably, at least 60%, 65% or 70% of the constituent nucleotides in the RNA molecule are modified, wherein the modified nucleotides may comprise adenine, cytosine, guanine and/or uracil. Still more preferably, at least 75%, 80% or 85% of the constituent nucleotides in the RNA molecule are modified, wherein the modified nucleotides may comprise adenine, cytosine, guanine and/or uracil. Most preferably, at least 90%, 95%, 96%, 97%, 98%, 99% or 100% of the constituent nucleotides in the RNA molecule are modified, wherein the modified nucleotides may comprise adenine, cytosine, guanine and/or uracil.

As mentioned above, the inventors have measured the enhancement of RNA translation in terms of luciferase activity instead of directly measuring the amount of luciferase protein that is produced. As such, luciferase activity may be considered as a surrogate of RNA translation.

Suitably, the one or more modified NTP (preferably, 2′-O-methyl NTP) enhances the level of expression and/or translation of the RNA molecule by at least 5-fold, 10-fold or 2-fold increase compared to the level of expression and/or translation that occurs in the absence of the one or more modified NTP. More suitably, the one or more modified NTP (preferably, 2′-O-methyl NTP) enhances the level of expression and/or translation of the RNA molecule by at least 5-fold, 10-fold or 50-fold compared to the level of expression and/or translation that occurs in the absence of the one or more modified NTP. Even more suitably, the one or more modified NTP (preferably, 2′-O-methyl NTP) enhances the level of expression and/or translation of the RNA molecule by at least 75-fold, 100-fold or 250-fold compared to the level of expression and/or translation that occurs in the absence of the one or more modified NTP. Preferably, the one or more modified NTP (preferably, 2′-O-methyl NTP) enhances the level of expression and/or translation of the RNA molecule by at least 500-fold, 750-fold or 1000-fold compared to the level of expression and/or translation that occurs in the absence of the one or more modified NTP. Preferably, the one or more modified NTP (preferably, 2′-O-methyl NTP) enhances the level of expression and/or translation of the RNA molecule by at least 2000-fold, 5000-fold or 8000-fold increase compared to the level of expression and/or translation that occurs in the absence of the one or more modified NTP. As shown in FIG. 28, in some embodiments (e.g. Gm and Cm for mRNA), some cells are surprisingly exhibiting a 1000-8000-fold increase in expression.

The level of expression and/or translation may be measured by the expression of a fluorescent or luminescent protein encoded as a gene of interest in the RNA molecule (e.g. in either mRNA or saRNA). For example, the luminescent protein may be a bioluminescent protein, such as a luciferase (e.g. firefly luciferase protein encoded as a gene of interest). Thus, expression of luciferase can be determined by measurement of its enzymatic activity, where luciferin is converted to oxyluciferin emitting light that can be quantitated as relative light units (RLUs). The presence of light is proportional to the amount of translated protein. This approach was previously used to assess the impact of pseudouridine incorporation on translational efficiency [19].

The enzymatic incorporation of modified NTPs (preferably, 2′-O-methyl NTP) in RNA has many potential outcomes and advantages selected from a group consisting of: changing mRNA stability; preventing innate recognition; reducing RNA degradation; increasing RNA half-life; enhancing recruitment of translational factors and/or ribosomes; and increasing translation.

Preferably, the methods or uses enhance protein expression from mRNA, saRNA, circular RNA or any RNA sequence capable to being translated to generate a recombinant protein or peptide.

In another embodiment, the methods or uses enhance the stability of RNA (be it mRNA, miRNA; siRNA; shRNA; anti-sense RNA; RNA aptamers; self-amplifying RNA (saRNA); interference RNA (RNAi); non-coding RNA; circular RNA; or small RNA). Improved stability provides a significant advantage to both coding and non-coding biotherapeutic or prophylatic uses of RNA molecules. Previous studies have shown that incorporation of 2′-O-methylated NTPs enhances the stability of short RNA oligonucleotides, aptamers and iRNA [20, 21]. In this respect, 2′-O-methylation increases the RNA resistance to degradation due to the abolishment of the hydrogen bonding and nucleophilic characters of 2′-OH. This leads to highly increased stability of 2′-O-methylated RNA against alkaline (and even enzymatic) hydrolysis. Furthermore, 2′-O methylation increases base pairing strength and stability of alternative RNA conformations [22]. Although evaluated for short RNAi and aptamers, this has never been evaluated for mRNA or saRNA as a methodology for incorporation of 2′-O-methylated NTPs in RNA sequences of greater than >100 bp did not exist.

Incorporation of 2′-O-methylated NTPs in short RNA sequences was achieved either by chemical synthesis or in vitro transcription, the latter being very inefficient. The methods described herein, however, allow the incorporation of modified NTPs (preferably, 2′-O-methyl NTP) of any length (be they coding or non-coding) providing enhanced stability of the resultant RNA molecule.

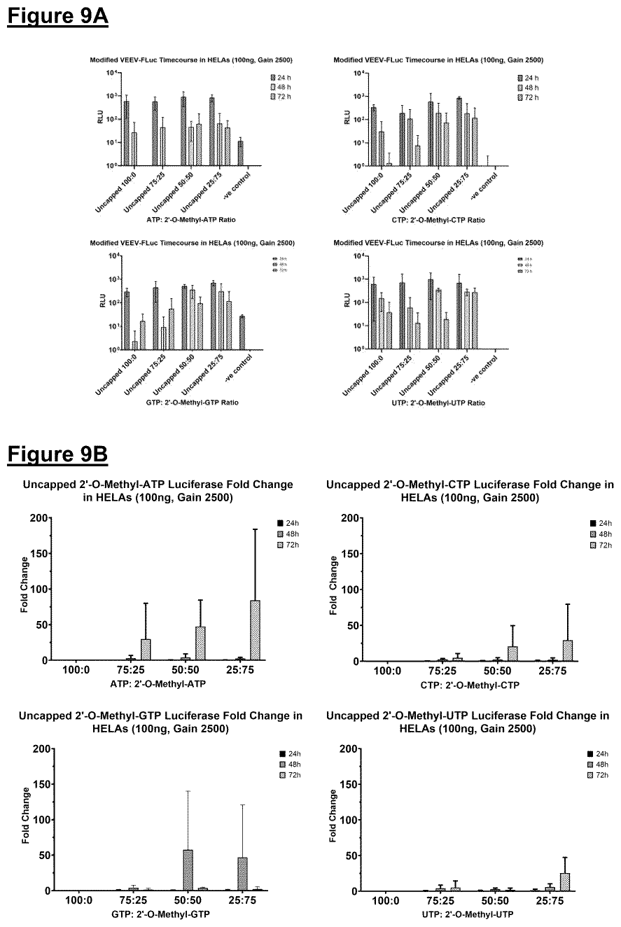

Stability of RNA molecules can be measured by quantitative PCR, but is also reflected by the duration of expression, where faster degradation equates to shorter expression. The inventors observe a prolonged duration of expression for saRNA containing 75% 2-O-methyl GTP over unmodified GTP out to 72 h (see FIGS. 9-13) and for mRNA containing 2-O-methyl modified GTP (Gm) plus CTP (Cm) (see FIGS. 25-28).

In another embodiment, the methods or uses result in reducing or eliminating innate sensing of RNA by membrane, endosomal and cellular RNA binding proteins that recognise unmodified RNA structures that trigger interferon signalling pathways and activation of cytoplasmic nucleases that degrade RNA such as RNase L [23]. Reduced innate sensing of RNA (be it mRNA, miRNA; siRNA; shRNA; anti-sense RNA; RNA aptamers; self-amplifying RNA (saRNA); interference RNA (RNAi); non-coding RNA; circular RNA; and small RNA) would provide significant advantages to both coding and non-coding biotherapeutic or prophylatic uses of RNA. Previous studies have shown that incorporation of 2′-O-methylated NTPs affects innate recognition, interferon induction and associated RNA degradation of short RNA oligonucleotides, aptamers and iRNA [24, 25, 26, 27]. This, however, has not been evaluated for mRNA or saRNA as a methodology for incorporation of 2′-O-methylated NTPs in RNA sequences of greater than >100 bp. Incorporation of 2′-O-methylated NTPs in short RNA sequences was achieved either by chemical synthesis or in vitro transcription, the latter being very inefficient. The methods presented here allow incorporation of 2′-O-methylated NTPs of any length to prevent innate recognition of RNA, interferon induction and RNA degradation.

Reducing the activation of innate sensing, leading to degradation of an RNA molecule is inferred by improved expression of modified saRNA and mRNA in interferon competent cells (HeLa and THP-1 cells). Although the incorporation of 2-O-methyl modified bases provides some improvement in expression in HEK293T cells, these cells have impaired innate sensing mechanisms, therefore, any improvement in expression directly relates to improved RNA half-life in the absence of innate recognition. Expression of unmodified saRNA and mRNA, if greatly diminished in both HeLa and THP-1 cells, reflects enhanced degradation caused by the triggering of innate sensing mechanisms. The incorporation of 2′-O-methylated NTPs in saRNA and mRNA provides more pronounced increase of expression in these two interferon-competent cell types (fold change) than that observed in HEK293T cells. Induction of type I interferon expression can be measured by ELISA as a surrogate of innate activation.

It will be appreciated that the composition, the pharmaceutical composition, the vaccine or the therapeutic RNA molecule, may be used in a medicament, which may be used as a monotherapy (i.e. use of the composition alone). Alternatively, the composition according to the invention may be used as an adjunct to, or in combination with, known therapies for treating, ameliorating, or preventing an infection or disease.

The composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, polyplex, emulsion, liposome, lipid nanoparticles, functionalised liposomes or lipid nanoparticles (e.g. with peptide, antibody, antibody fragment, glycan, glyco-conjugate, DNA or RNA on the surface or encapsulated) or any other suitable form that may be administered to a person or animal. The lipid nanoparticle may comprise one or more components selected from a group consisting of: a cationic lipid (which is preferably ionisable); phosphatidylcholine; cholesterol; and polyethylene glycol (PEG)-lipid. It will be appreciated that the vehicle of medicaments according to the invention should be one which is well-tolerated by the subject to whom it is given.

Medicaments comprising the composition, the pharmaceutical composition or the vaccine of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin. Formulations may be provided in a lyophilised format for reconstitution or as a solid dosage form for topical, inserted or injected delivery.

The composition, the pharmaceutical composition, vaccine or therapeutic RNA molecule of the invention may also be incorporated within a slow- or delayed-release device. Such devices may, for example, be inserted on or under the skin, and the medicament may be released over weeks or even months. The device may be located at least adjacent the treatment site.

In a preferred embodiment, however, medicaments according to the invention may be administered to a subject by injection into the blood stream, muscle, skin or directly into a site requiring treatment. Injections may be intravenous (bolus or infusion), subcutaneous (bolus or infusion), intradermal (bolus or infusion), intramuscular (bolus or infusion), intrathecal (bolus or infusion), epidural (bolus or infusion) or intraperitoneal (bolus or infusion).

It will be appreciated that the amount of composition, the pharmaceutical composition or the vaccine or therapeutic RNA molecule that is required is determined by its biological activity and bioavailability, which in turn depends on the mode of administration, the physiochemical properties of the composition, the pharmaceutical composition or the vaccine and whether it is being used as a monotherapy or in a combined therapy.

The frequency of administration will also be influenced by the half-life of the active agent within the subject being treated. Optimal dosages to be administered may be determined by those skilled in the art, and will vary with the composition, the pharmaceutical composition or the vaccine in use, the strength of the pharmaceutical composition, the mode of administration, and the type of treatment. Additional factors depending on the particular subject being treated will result in a need to adjust dosages, including subject age, weight, gender, diet, and time of administration.

The required dose may depend upon a number of factors including, but not limited to, the active agent being administered, the disease being treated and/or vaccinated against, the subject being treated, etc.

Generally, a dose of between 0.001 μg/kg of body weight and 10 mg/kg of body weight, or between 0.01 μg/kg of body weight and 1 mg/kg of body weight, of the composition, the pharmaceutical composition or the vaccine or therapeutic RNA molecule of the invention may be used, depending upon the active agent used. A dose may be understood to relate to the quantity of the RNA molecule which is delivered.

Doses may be given as a single administration (e.g., a single injection). Alternatively, the composition, the pharmaceutical composition, biotherapeutic RNA molecule or the vaccine may require more than one administration. As an example, the composition, the pharmaceutical composition or the vaccine or therapeutic RNA molecule may be administered as an initial primer and a subsequent boost(s), or two boosts administered at between a week or monthly intervals (e.g. two or more doses of between 0.07 μg and 700 mg, i.e., assuming a body weight of 70 kg), or for a biotherapeutic repeat dosing on a daily, weekly or monthly basis. Alternatively, a slow-release device may be used to provide optimal doses of the composition, the pharmaceutical composition or the vaccine or therapeutic RNA molecule according to the invention to a patient without the need to administer repeated doses. Routes of administration may incorporate intravenous, intradermal subcutaneous, intramuscular, intrathecal, epidural or intraperitoneal routes of injection.

Known procedures, such as those conventionally employed by the pharmaceutical industry (e.g., in vivo experimentation, clinical trials, etc.), may be used to form specific formulations of the composition, the pharmaceutical composition or vaccine or therapeutic RNA molecule according to the invention and precise therapeutic regimes (such as doses of the agents and the frequency of administration).

A “subject” may be a vertebrate, mammal, or domestic animal. Hence, compositions and medicaments according to the invention may be used to treat any mammal, for example livestock (e.g., a horse), pets, or may be used in other veterinary applications. Most preferably, however, the subject is a human being.

A “therapeutically effective amount” of the composition, the pharmaceutical composition or the vaccine or therapeutic RNA molecule is any amount which, when administered to a subject, is the amount of the aforementioned that is needed to produce a therapeutic effect.

For example, a therapeutically effective amount of the composition, the pharmaceutical composition and the vaccine or therapeutic RNA molecule of the invention may comprise from about 0.0001 mg to about 800 mg of the payload molecule, preferably 0.001 mg to about 650 mg, and preferably from about 0.01 mg to about 500 mg of the payload molecule.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g., composition of the invention) may be mixed with a vehicle having the necessary compression properties in suitable proportions and compacted in the shape and size desired. The pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The composition according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and subcutaneous injection. The composition of the invention may be prepared as any appropriate sterile injectable medium.

The composition and/or the pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The composition of the invention and/or the pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

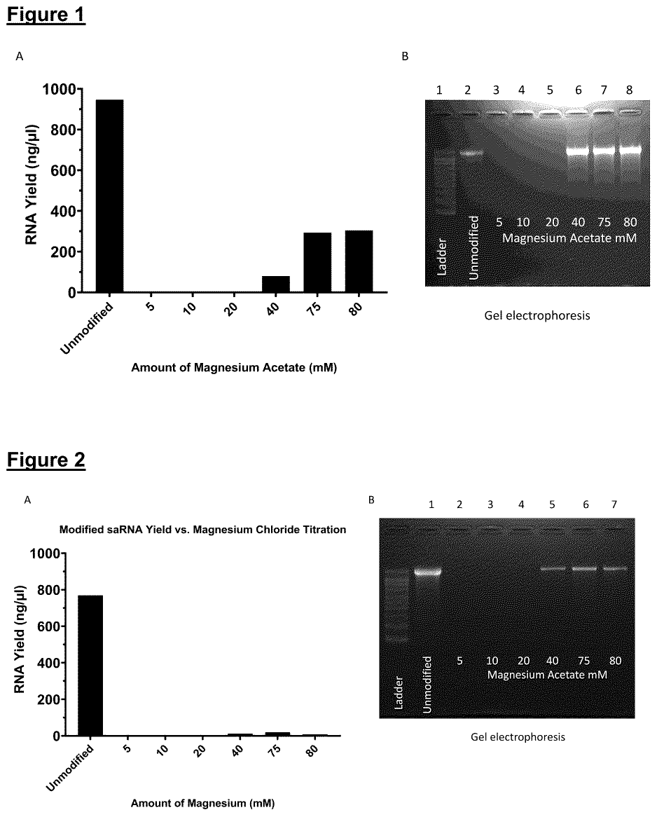

FIG. 1 shows that the efficient incorporation of 2′methyl-ribose modified NTPs only occurs at magnesium levels >20 nm when using magnesium acetate. A) RNA yield ng/ul, B) RNA product as assessed by gel-electrophoresis.

FIG. 2 shows that low or no incorporation of 2′methyl-ribose modified GTP occurs when using magnesium chloride. A) RNA yield ng/ul, B) RNA product as assessed by gel-electrophoresis.

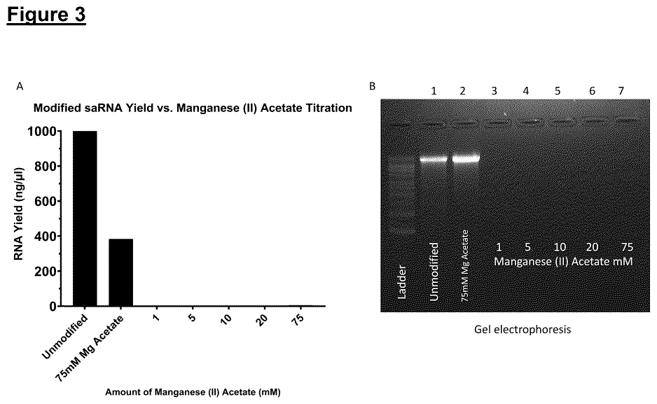

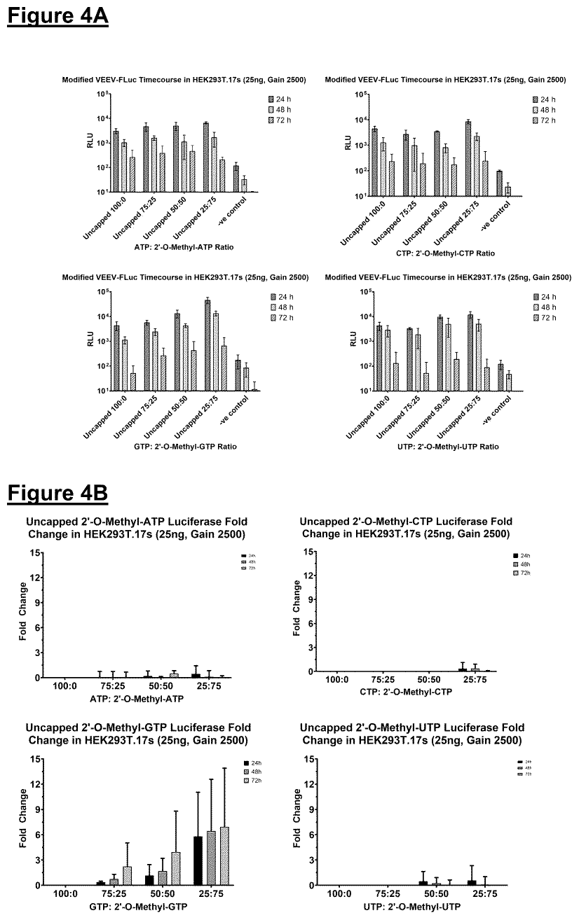

FIG. 3 illustrates that no detectable incorporation of 2′methyl-ribose modified GTP is observed when using magnesium acetate is replaced by manganese acetate. A) RNA yield ng/ul, B) RNA product as assessed by gel-electrophoresis.

FIG. 4 illustrates the impact of 2′-O-methyl NTP substitution on uncapped saRNA expression of fLuc. Uncapped saRNA encoding fLuc was generated by IVT, where individual nucleotides were substituted with 25, 50 or 75% 2′-O-methyl modified versions. HEK293T cells were transfected with 25 ng of saRNA formulated in lipofectamine. A). Luciferase expression was monitored over 72 h by measurement of relative light units (see methods). B) Fold change in expression relative to unmodified saRNA.

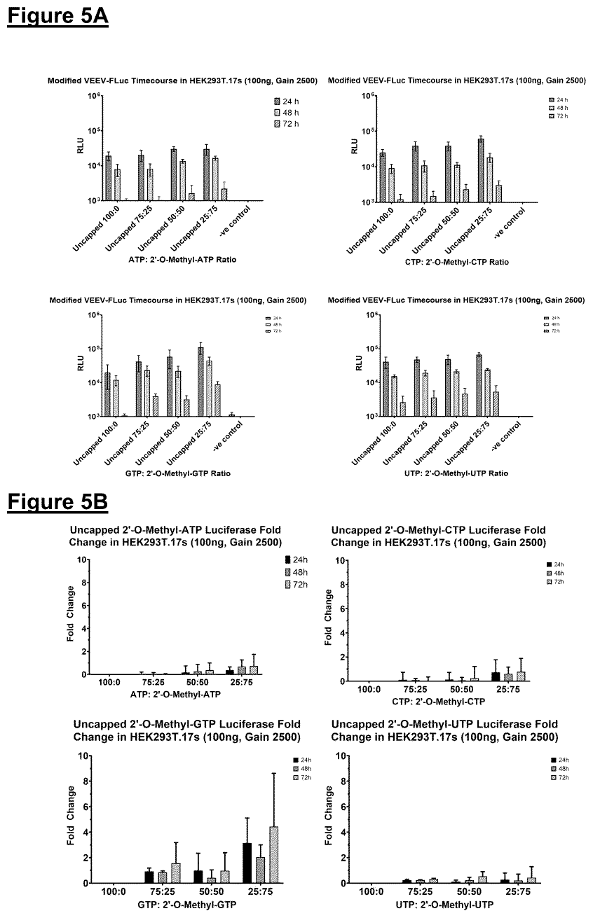

FIG. 5 shows the impact of 2′-O-methyl NTP substitution on uncapped saRNA expression of fLuc. Uncapped saRNA encoding fLuc was generated by IVT, where individual nucleotides were substituted with 25, 50 or 75% 2′-O-methyl modified versions. HEK293T cells were transfected with 100 ng of saRNA formulated in lipofectamine. A). Luciferase expression was monitored over 72 h by measurement of relative light units (see methods). B) Fold change in expression relative to unmodified saRNA.

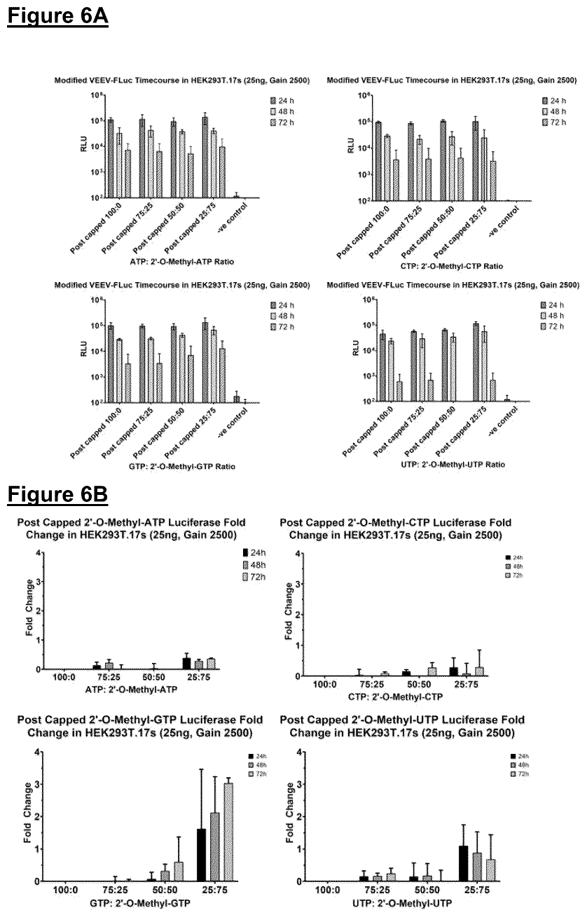

FIG. 6 illustrates the impact of 2′-O-methyl NTP substitution on 5′ capped saRNA expression of fLuc. 5′ capped saRNA encoding fLuc was generated by IVT, where individual nucleotides were substituted with 25, 50 or 75% 2′-O-methyl modified versions. HEK293T cells were transfected with 25 ng of saRNA formulated in lipofectamine. A). Luciferase expression was monitored over 72 h by measurement of relative light units (see methods). B) Fold change in expression relative to unmodified saRNA.

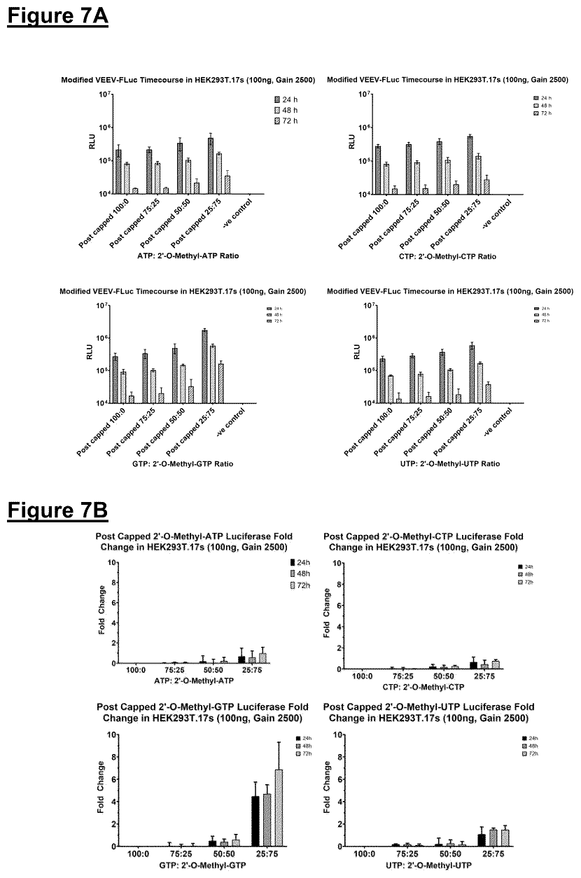

FIG. 7 illustrates the impact of 2′-O-methyl NTP substitution on 5′ capped saRNA expression of fLuc. Capped saRNA encoding fLuc was generated by IVT, where individual nucleotides were substituted with 25, 50 or 75% 2′-O-methyl modified versions. HEK293T cells were transfected with 100 ng of saRNA formulated in lipofectamine. A). Luciferase expression was monitored over 72 h by measurement of relative light units (see methods). B) Fold change in expression relative to unmodified saRNA.

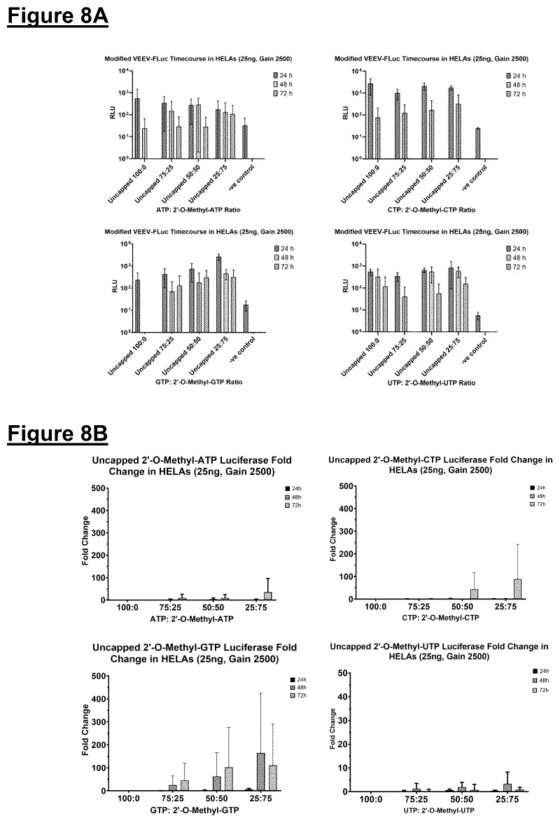

FIG. 8 shows the impact of 2′-O-methyl NTP substitution on uncapped saRNA expression of fLuc in HeLa cells. Uncapped saRNA encoding fLuc was generated by IVT, where individual nucleotides were substituted with 25, 50 or 75% 2′-O-methyl modified versions. HeLa cells were transfected with 25 ng of saRNA formulated in lipofectamine. A). Luciferase expression was monitored over 72 h by measurement of relative light units (see methods). B) Fold change in expression relative to unmodified saRNA.

FIG. 9 illustrates the impact of 2′-O-methyl NTP substitution on uncapped saRNA expression of fLuc in HeLa cells. Uncapped saRNA encoding fLuc was generated by IVT, where individual nucleotides were substituted with 25, 50 or 75% 2′-O-methyl modified versions. HeLa cells were transfected with 100 ng of saRNA formulated in lipofectamine. A). Luciferase expression was monitored over 72 h by measurement of relative light units (see methods). B) Fold change in expression relative to unmodified saRNA.

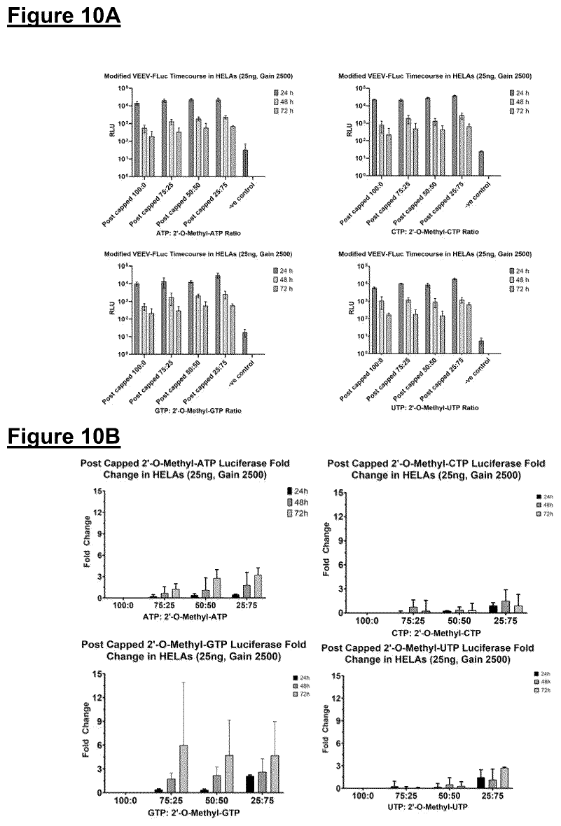

FIG. 10 illustrates the impact of 2′-O-methyl NTP substitution on 5′capped saRNA expression of fLuc in HeLa cells. 5′capped saRNA encoding fLuc was generated by IVT, where individual nucleotides were substituted with 25, 50 or 75% 2′-O-methyl modified versions. HeLa cells were transfected with 25 ng of saRNA formulated in lipofectamine. A). Luciferase expression was monitored over 72 h by measurement of relative light units (see methods). B) Fold change in expression relative to unmodified saRNA.

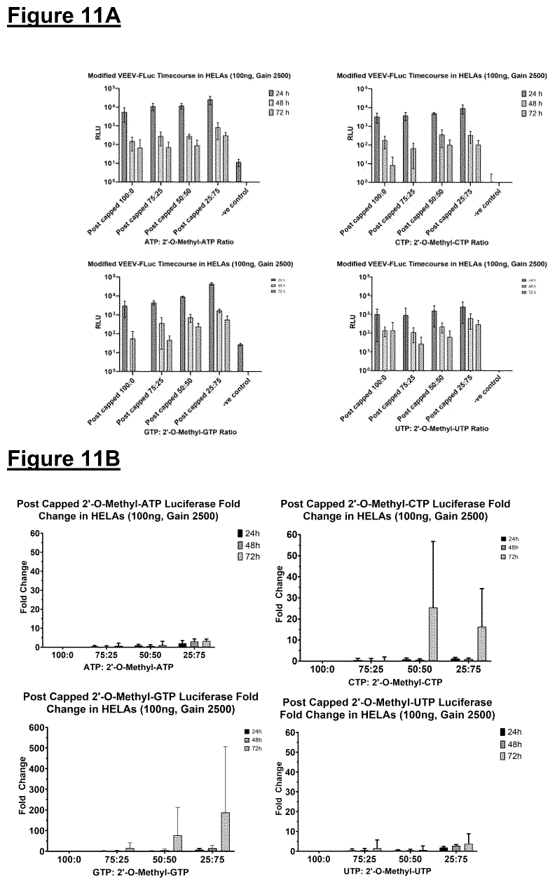

FIG. 11 illustrates the impact of 2′-O-methyl NTP substitution on 5′capped saRNA expression of fLuc in HeLa cells. 5′capped saRNA encoding fLuc was generated by IVT, where individual nucleotides were substituted with 25, 50 or 75% 2′-O-methyl modified versions. HeLa cells were transfected with 100 ng of saRNA formulated in lipofectamine. A). Luciferase expression was monitored over 72 h by measurement of relative light units (see methods). B) Fold change in expression relative to unmodified saRNA.

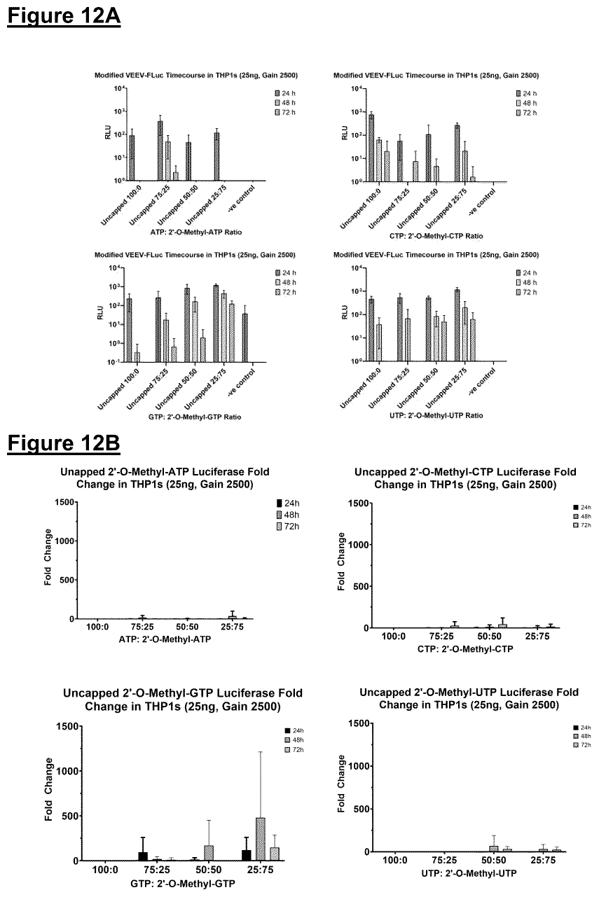

FIG. 12 shows the impact of 2′-O-methyl NTP substitution on uncapped saRNA expression of fLuc in THP-1 cells. Uncapped saRNA encoding fLuc was generated by IVT, where individual nucleotides were substituted with 25, 50 or 75% 2′-O-methyl modified versions. THP-1 cells were transfected with 25 ng of uncapped saRNA formulated in lipofectamine. A). Luciferase expression was monitored over 72 h by measurement of relative light units (see methods). B) Fold change in expression relative to unmodified saRNA.

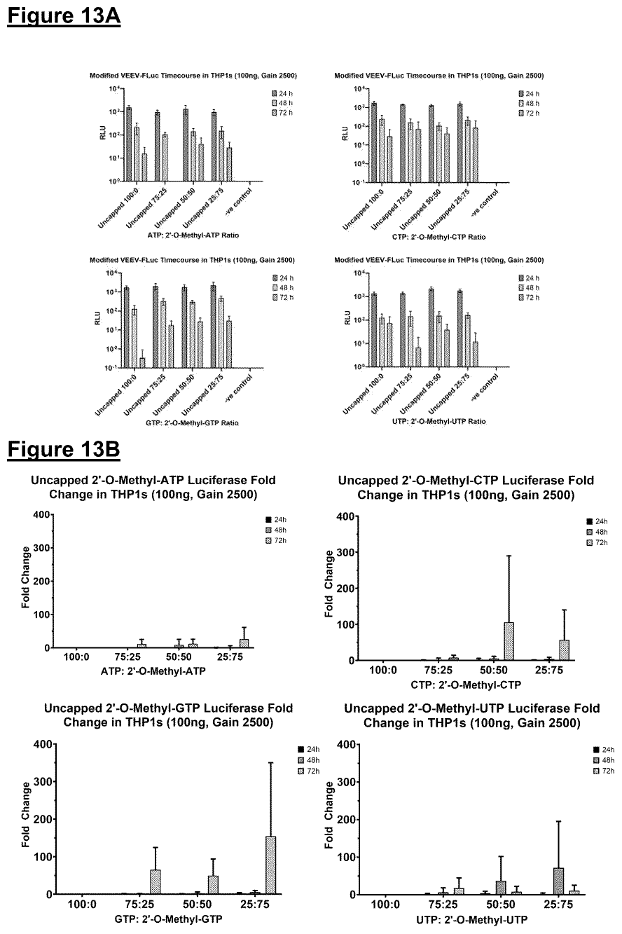

FIG. 13 shows the impact of 2′-O-methyl NTP substitution on uncapped saRNA expression of fLuc in THP-1 cells. Uncapped saRNA encoding fLuc was generated by IVT, where individual nucleotides were substituted with 25, 50 or 75% 2′-O-methyl modified versions. THP-1 cells were transfected with 100 ng of uncapped saRNA formulated in lipofectamine. A). Luciferase expression was monitored over 72 h by measurement of relative light units (see methods). B) Fold change in expression relative to unmodified saRNA.

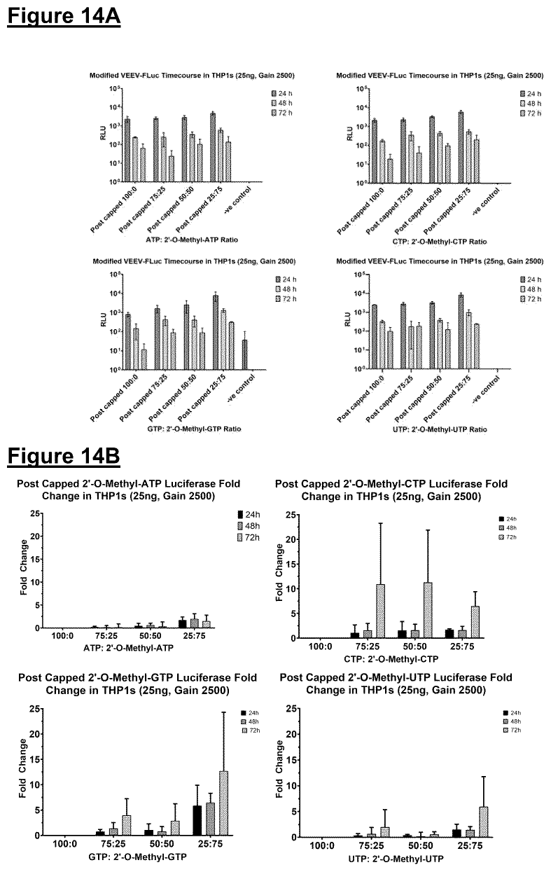

FIG. 14 shows the impact of 2′-O-methyl NTP substitution on 5′capped saRNA expression of fLuc in THP-1 cells. 5′capped saRNA encoding fLuc was generated by IVT, where individual nucleotides were substituted with 25, 50 or 75% 2′-O-methyl modified versions. THP-1 cells were transfected with 25 ng of uncapped saRNA formulated in lipofectamine. A). Luciferase expression was monitored over 72 h by measurement of relative light units (see methods). B) Fold change in expression relative to unmodified saRNA.

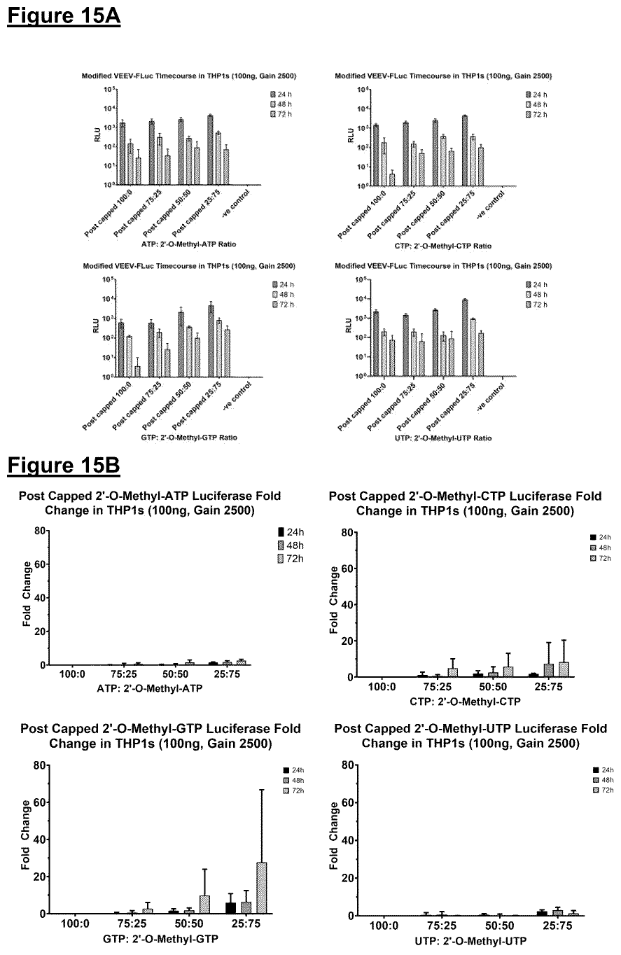

FIG. 15 shows the impact of 2′-O-methyl NTP substitution on 5′capped saRNA expression of fLuc in THP-1 cells. 5′capped saRNA encoding fLuc was generated by IVT, where individual nucleotides were substituted with 25, 50 or 75% 2′-O-methyl modified versions. THP-1 cells were transfected with 25 ng of uncapped saRNA formulated in lipofectamine. A). Luciferase expression was monitored over 72 h by measurement of relative light units (see methods). B) Fold change in expression relative to unmodified saRNA.

FIG. 16 shows the impact of N1 methyl pseudouridine substitution of uncapped and 5′capped saRNA expression of fLuc in A) HEK293T cells and B) HeLa cells. Uncapped and 5′capped saRNA encoding fLuc was generated by IVT, where unmodified UTP was substituted with 25, 50 or 75% N1 methyl pseudouridine. Cells were transfected with 100 ng of uncapped or capped saRNA formulated in lipofectamine and luciferase expression was monitored over 72 h by measurement of relative light units (see methods).

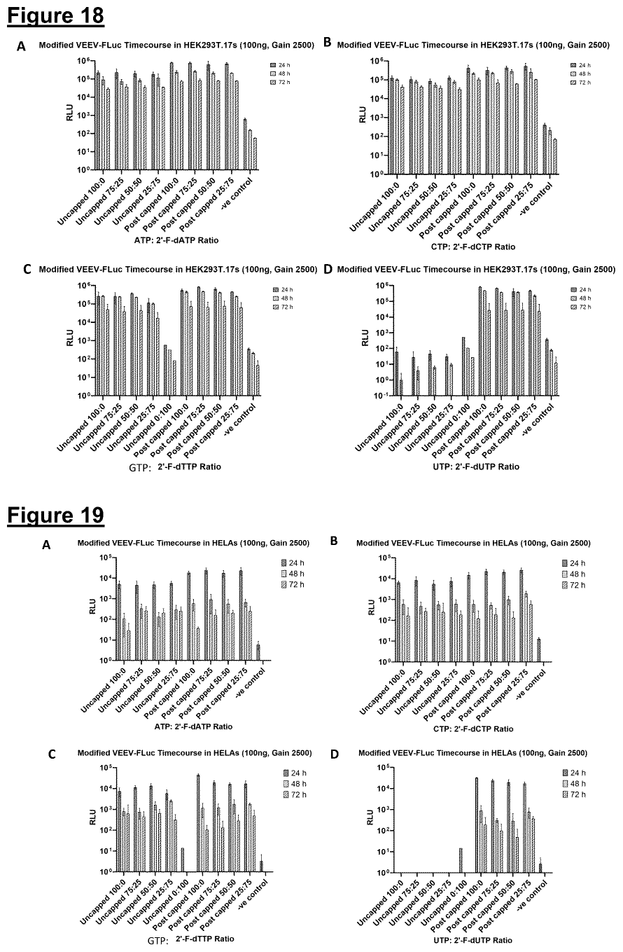

FIG. 17 shows the impact of 7-deaza-GTP substitution of uncapped and 5′capped saRNA expression of fLuc in A) HEK293T cells and B) HeLa cells. Uncapped and 5′capped saRNA encoding fLuc was generated by IVT, where unmodified GTP was substituted with 25, 50 or 75% 7-deaza-GTP. Cells were transfected with 100 ng of uncapped or capped saRNA formulated in lipofectamine and luciferase expression was monitored over 72 h by measurement of relative light units (see methods).

FIG. 18 shows the impact of 2′-fluoro substitutions of uncapped and 5′capped saRNA expression of fLuc in HEK293T cells. Uncapped and 5′capped saRNA encoding fLuc was generated by IVT, where unmodified bases were substituted with 25, 50 or 75% 2′Fluoro-ATP, CTP, or UTP. Cells were transfected with 100 ng of uncapped or capped saRNA formulated in lipofectamine and luciferase expression was monitored over 72 h by measurement of relative light units (see methods).

FIG. 19 shows the impact of 2′-fluoro substitutions of uncapped and 5′capped saRNA expression of fLuc in HeLa cells. Uncapped and 5′capped saRNA encoding fLuc was generated by IVT, where unmodified bases were substituted with 25, 50 or 75% 2′Fluoro-ATP, CTP, or UTP. Cells were transfected with 100 ng of uncapped or capped saRNA formulated in lipofectamine and luciferase expression was monitored over 72 h by measurement of relative light units (see methods).

FIG. 20 shows the impact of 2′-O-methyl NTP substitution on 5′capped saRNA and mRNA expression of fLuc in HEK293T cells. 5′capped saRNA and mRNA encoding fLuc was generated by IVT. Expression of unmodified saRNA and mRNA was compared to saRNA and mRNA where 75% of GTP was substituted with 75% 2′-O-methyl GTP versions. In addition, “combo” mRNA was generated by substitution of 75% of GTP and ATP and 90% of CTP and UTP with 2′-O-methyl versions. HEK293T cells were transfected with 100 ng of 5′capped saRNA and mRNA formulated in lipofectamine. A). Luciferase expression was monitored over 72 h by measurement of relative light units. B) Fold change in expression relative to unmodified saRNA.

FIG. 21 shows the impact of 2′-O-methyl NTP substitution on 5′capped saRNA and mRNA expression of fLuc in Hela cells. 5′capped saRNA and mRNA encoding fLuc was generated by IVT. Expression of unmodified saRNA and mRNA was compared to saRNA and mRNA where 75% of GTP was substituted with 75% 2′-O-methyl GTP versions. In addition, “combo” mRNA was generated by substitution of 75% of GTP and ATP and 90% of CTP and UTP with 2′-O-methyl versions. Hela cells were transfected with 100 ng of 5′capped saRNA and mRNA formulated in lipofectamine. A). Luciferase expression was monitored over 72 h by measurement of relative light units. B) Fold change in expression relative to unmodified saRNA.

FIG. 22 shows the impact of 2′-O-methyl NTP substitution on 5′capped saRNA and mRNA expression of fLuc in THP-1 cells. 5′capped saRNA and mRNA encoding fLuc was generated by IVT. Expression of unmodified saRNA and mRNA was compared to saRNA and mRNA where 75% of GTP was substituted with 75% 2′-O-methyl GTP versions. In addition, “combo” mRNA was generated by substitution of 75% of GTP and ATP and 90% of CTP and UTP with 2′-O-methyl versions. THP-1 cells were transfected with 100 ng of 5′capped saRNA and mRNA formulated in lipofectamine. A). Luciferase expression was monitored over 72 h by measurement of relative light units. B) Fold change in expression relative to unmodified saRNA.

FIG. 23 shows the impact of 2′-O-methyl NTP substitution on capped mRNA expression of fLuc in HEK293T cells. Capped mRNA encoding fLuc was generated by IVT, where nucleotides were substituted with ≥75% 2′-O-methyl modified versions. Combo condition is where all four NTPs were substituted. HEK293T cells were transfected with 25 ng of capped mRNA formulated in lipofectamine. Luciferase expression was monitored over 72 h by measurement of relative light units (see methods).

FIG. 24 shows the impact of 2′-O-methyl NTP substitution on capped mRNA expression of fLuc in HEK293T cells. Capped mRNA encoding fLuc was generated by IVT, where nucleotides were substituted with ≥75% 2′-O-methyl modified versions. Combo condition is where all four NTPs were substituted. HEK293T cells were transfected with 25 ng of capped mRNA formulated in lipofectamine. Luciferase expression was monitored over 72 h by measurement of relative light units and is represented as fold change in expression relative to unmodified saRNA.

FIG. 25 shows the impact of 2′-O-methyl NTP substitution on capped mRNA expression of fLuc in HeLa cells. Capped mRNA encoding fLuc was generated by IVT, where nucleotides were substituted with ≥75% 2′-O-methyl modified versions. Combo condition is where all four NTPs were substituted. HeLa cells were transfected with 25 ng of capped mRNA formulated in lipofectamine. Luciferase expression was monitored over 72 h by measurement of relative light units (see methods).

FIG. 26 shows the impact of 2′-O-methyl NTP substitution on capped mRNA expression of fLuc in HeLa cells. Capped mRNA encoding fLuc was generated by IVT, where nucleotides were substituted with ≥75% 2′-O-methyl modified versions. Combo condition is where all four NTPs were substituted. HeLa cells were transfected with 25 ng of capped mRNA formulated in lipofectamine. Luciferase expression was monitored over 72 h by measurement of relative light units and is represented as fold change in expression relative to unmodified saRNA.

FIG. 27 shows the impact of 2′-O-methyl NTP substitution on capped mRNA expression of fLuc in THP-1 cells. Capped mRNA encoding fLuc was generated by IVT, where nucleotides were substituted with ≥75% 2′-O-methyl modified versions. Combo condition is where all four NTPs were substituted. THP-1 cells were transfected with 25 ng of capped mRNA formulated in lipofectamine. Luciferase expression was monitored over 72 h by measurement of relative light units (see methods).

FIG. 28 shows the impact of 2′-O-methyl NTP substitution on capped mRNA expression of fLuc in THP-1 cells. Capped mRNA encoding fLuc was generated by IVT, where nucleotides were substituted with ≥75% 2′-O-methyl modified versions. Combo condition is where all four NTPs were substituted. THP-1 cells were transfected with 25 ng of capped mRNA formulated in lipofectamine. Luciferase expression was monitored over 72 h by measurement of relative light units and is represented as fold change in expression relative to unmodified saRNA.

FIG. 29 shows the impact of 2′-O-methyl NTP substitution on capped mRNA expression of fLuc in HEK293T cells. Capped mRNA encoding fLuc was generated by IVT, where individual nucleotides were substituted with ≥75% 2′-O-methyl modified versions. Combo condition is where all four NTPs were substituted. HEK293T cells were transfected with 25 ng of capped mRNA formulated in lipofectamine. Luciferase expression was monitored over 72 h by measurement of relative light units (see methods).

FIG. 30 shows the impact of 2′-O-methyl NTP substitution on capped mRNA expression of fLuc in HEK293T cells. Capped mRNA encoding fLuc was generated by IVT, where individual nucleotides were substituted with ≥75% 2′-O-methyl modified versions. Combo condition is where all four NTPs were substituted. HEK293T cells were transfected with 100 ng of capped mRNA formulated in lipofectamine. Luciferase expression was monitored over 72 h by measurement of relative light units (see methods).

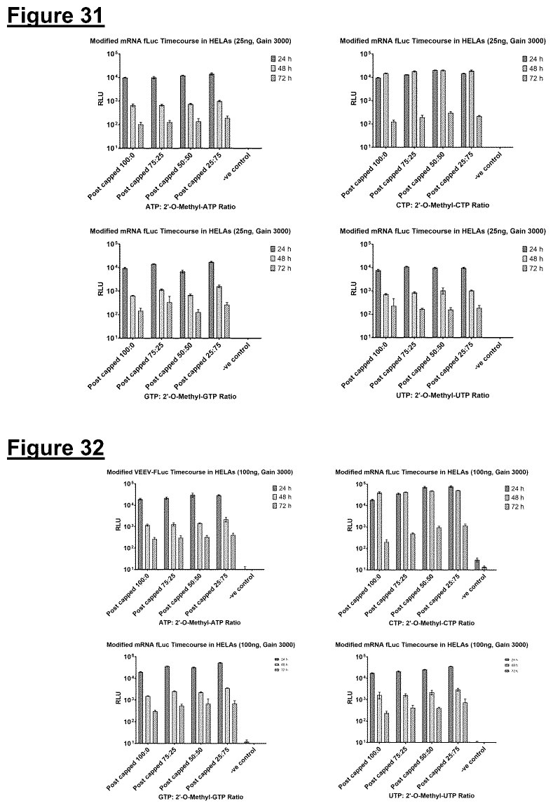

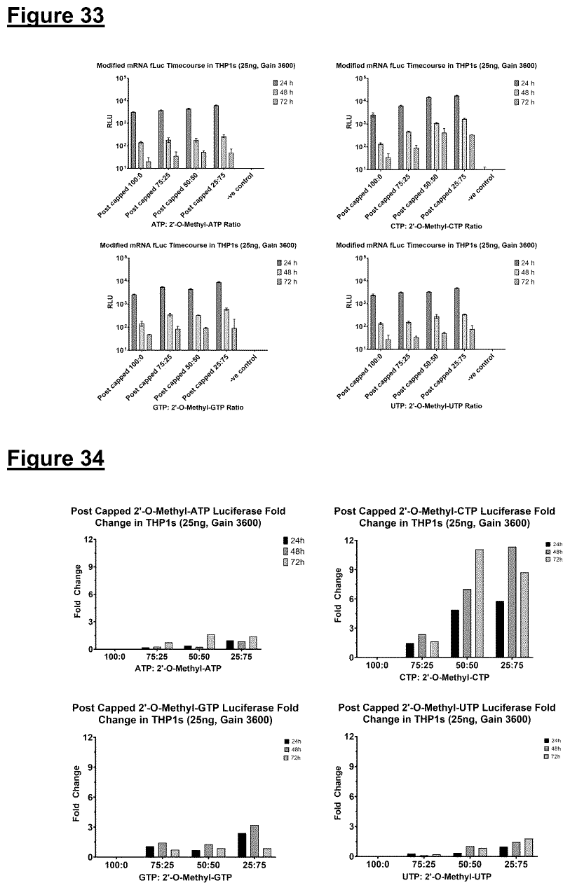

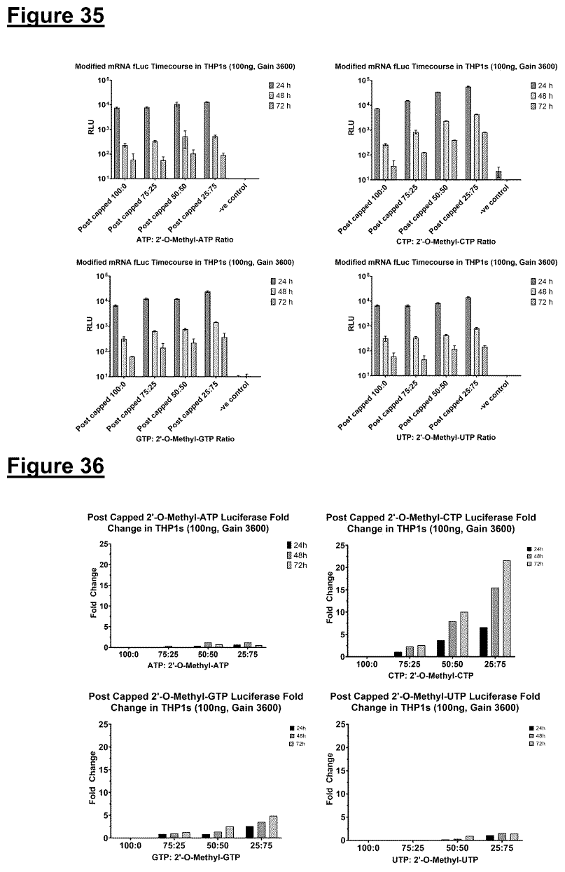

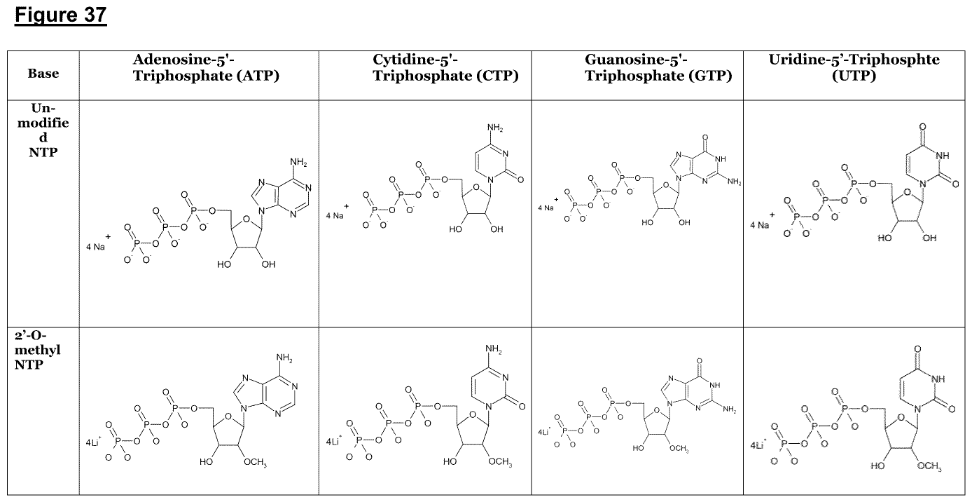

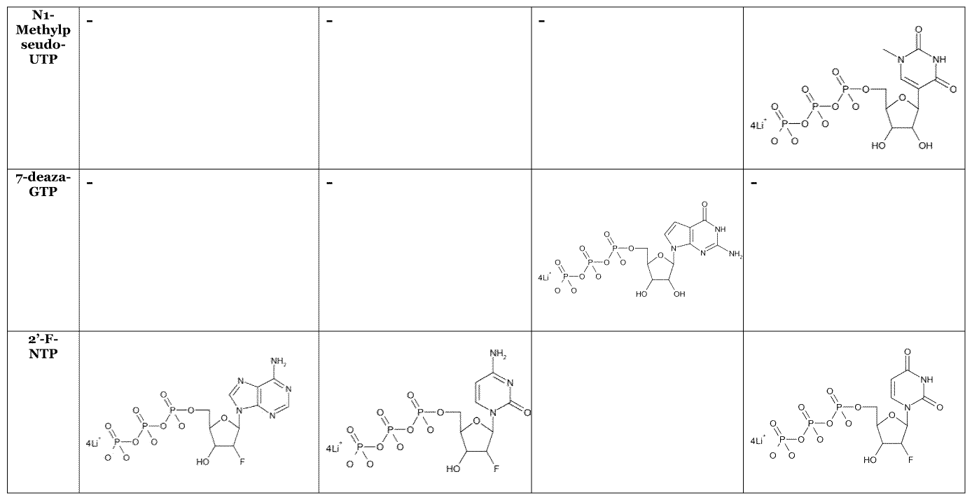

FIG. 31 shows the impact of 2′-O-methyl NTP substitution on capped mRNA expression of fLuc in HeLa cells. Capped mRNA encoding fLuc was generated by IVT, where individual nucleotides were substituted with ≥75% 2′-O-methyl modified versions. Combo condition is where all four NTPs were substituted. HeLa cells were transfected with 25 ng of capped mRNA formulated in lipofectamine. Luciferase expression was monitored over 72 h by measurement of relative light units (see methods).

FIG. 32 shows the impact of 2′-O-methyl NTP substitution on capped mRNA expression of fLuc in HeLa cells. Capped mRNA encoding fLuc was generated by IVT, where individual nucleotides were substituted with ≥75% 2′-O-methyl modified versions. Combo condition is where all four NTPs were substituted. HeLa cells were transfected with 100 ng of capped mRNA formulated in lipofectamine. Luciferase expression was monitored over 72 h by measurement of relative light units (see methods).

FIG. 34 shows the impact of 2′-O-methyl NTP substitution on capped mRNA expression of fLuc in THP-1 cells. Capped mRNA encoding fLuc was generated by IVT, where individual nucleotides were substituted with ≥75% 2′-O-methyl modified versions. Combo condition is where all four NTPs were substituted. THP-1 cells were transfected with 25 ng of capped mRNA formulated in lipofectamine. Luciferase expression was monitored over 72 h by measurement of relative light units (see methods) and is represented as fold change in expression relative to unmodified saRNA.

FIG. 35 shows the impact of 2′-O-methyl NTP substitution on capped mRNA expression of fLuc in THP-1 cells. Capped mRNA encoding fLuc was generated by IVT, where individual nucleotides were substituted with ≥75% 2′-O-methyl modified versions. Combo condition is where all four NTPs were substituted. THP-1 cells were transfected with 100 ng of capped mRNA formulated in lipofectamine. Luciferase expression was monitored over 72 h by measurement of relative light units (see methods).

FIG. 36 shows the impact of 2′-O-methyl NTP substitution on capped mRNA expression of fLuc in THP-1 cells. Capped mRNA encoding fLuc was generated by IVT, where individual nucleotides were substituted with ≥75% 2′-O-methyl modified versions. Combo condition is where all four NTPs were substituted. THP-1 cells were transfected with 100 ng of capped mRNA formulated in lipofectamine. Luciferase expression was monitored over 72 h by measurement of relative light units, shown as fold change over unmodified mRNA.

FIG. 37 is a table showing the structures of ATP, CTP, GTP and UTP as unmodified bases, or 2-O-methyl NTP. Also shown is N1-methyl-pseudo-UTP, 7-deaza-GTP, 2′fluoro-NTPs, including 2′fluoro-dTTP.

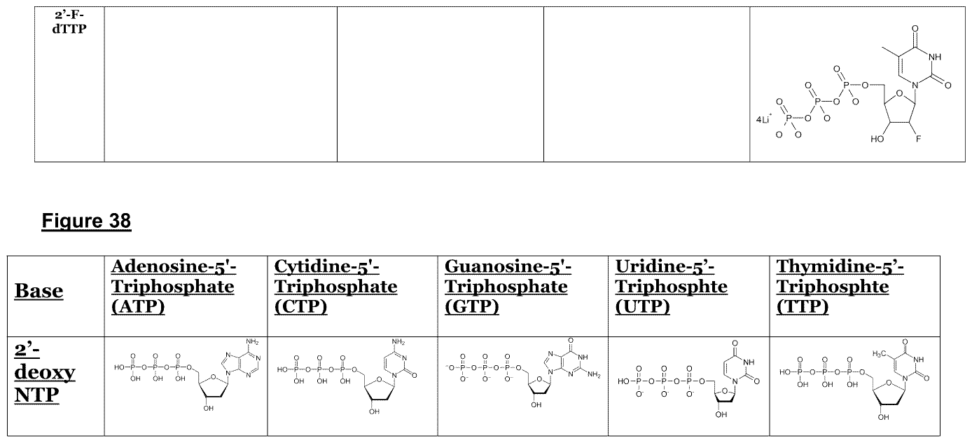

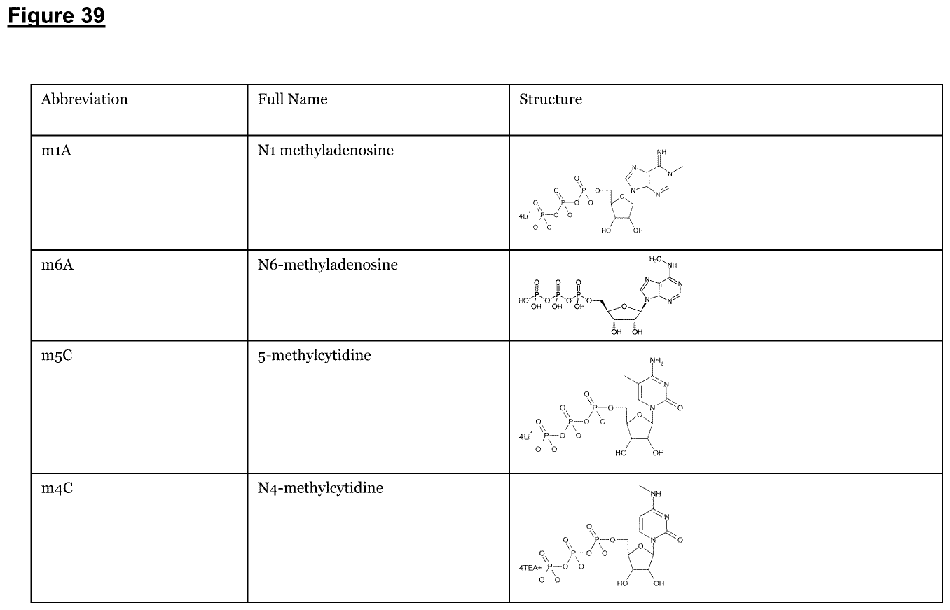

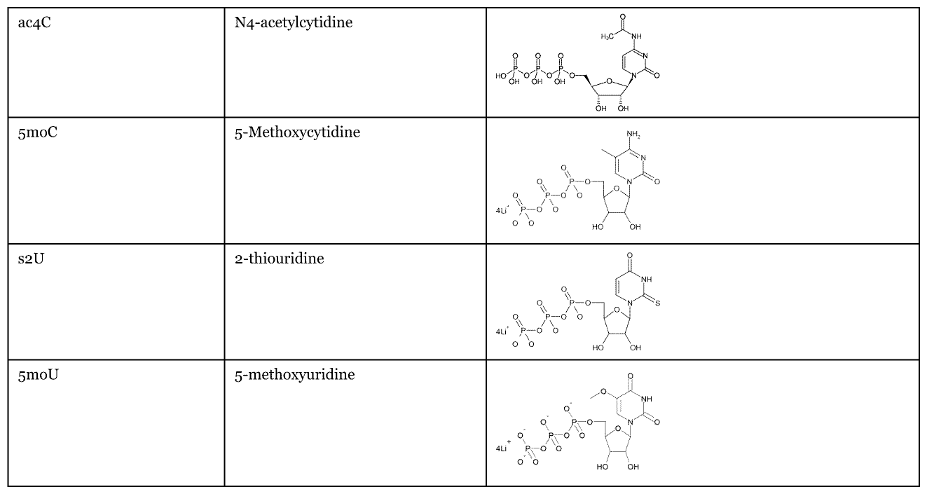

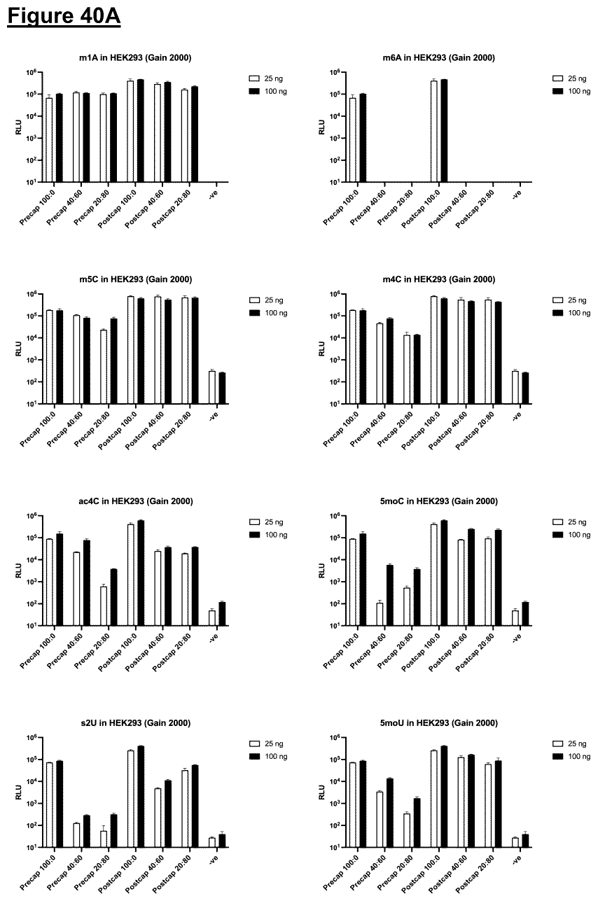

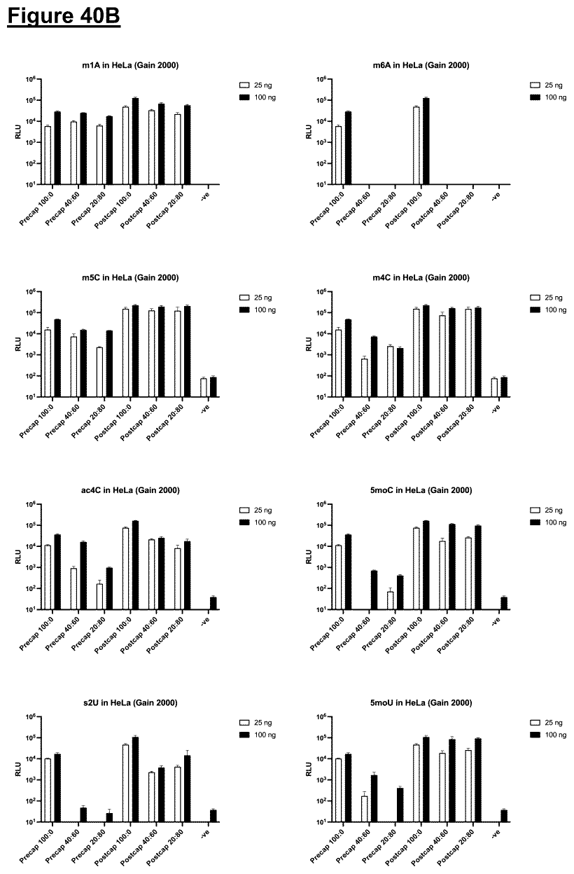

FIG. 38 is a table showing the structures of ATP, CTP, GTP and UTP as 2-deoxy NTPs, also including dTTP.

FIG. 39 shows the structures of alternative modified NTPs.

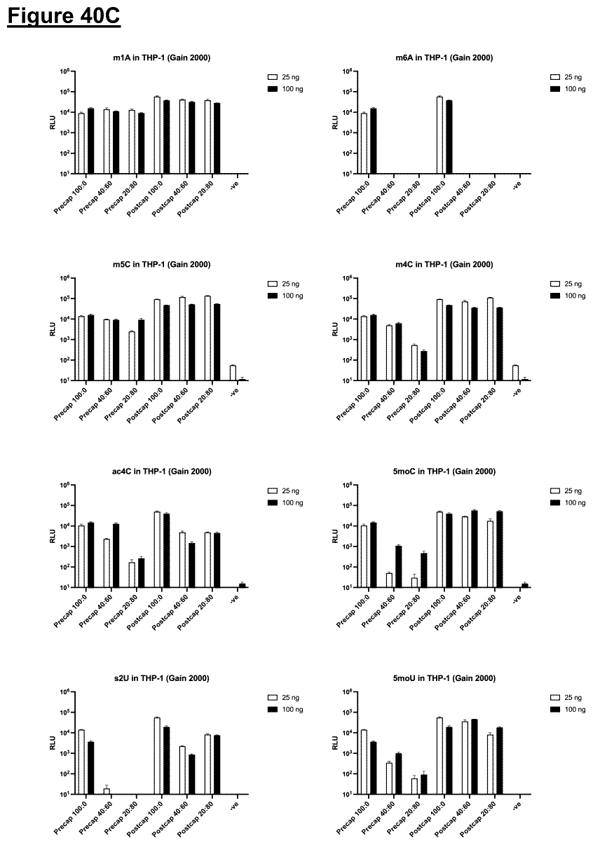

FIG. 40 shows the wide range of alternative modified NTPs that provide either no benefit or reduced expression when incorporated into saRNA and assessed for luciferase expression in Ao HEK293T, B) HeLa or C) THP-1 cells. Cells were transfected with 25 or 100 ng of uncapped (precapped) or capped saRNA formulated in lipofectamine. Luciferase expression was monitored over 24 h by measurement of relative light units, show as fold change over unmodified mRNA.

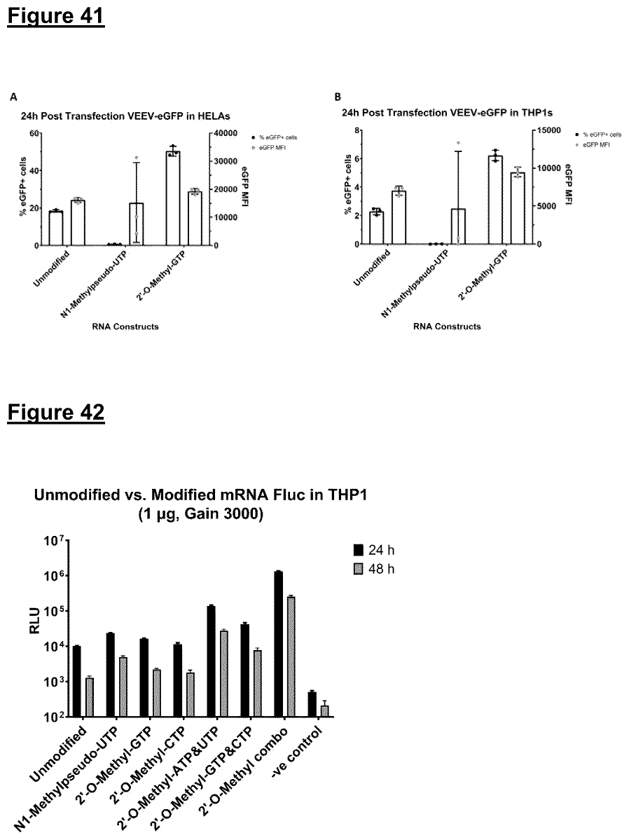

FIG. 41 shows the impact of 2′-O-methyl GTP substitution on capped saRNA expression of eGFP. Capped saRNA encoding eGFP was generated by IVT, using unmodified NTPs, where GTP was substituted with ≥75% 2′-O-methyl modified NTP, and where UTP was replaced with 100% N-1 methylpseudouridine. Unmodified and modified VEEV-eGFP saRNA were transfected in 24-well plates of (A) HELA and (B) THP1 cells at 1 μg in triplicates (Error bars represent n=3). 24 h post transfection, cells were harvested and stained before assessing for eGFP expression through flow cytometry. Data show the % of cells expressing eGFP (left vertical axis) and the Medium Fluorescent Intensity (right vertical axis).

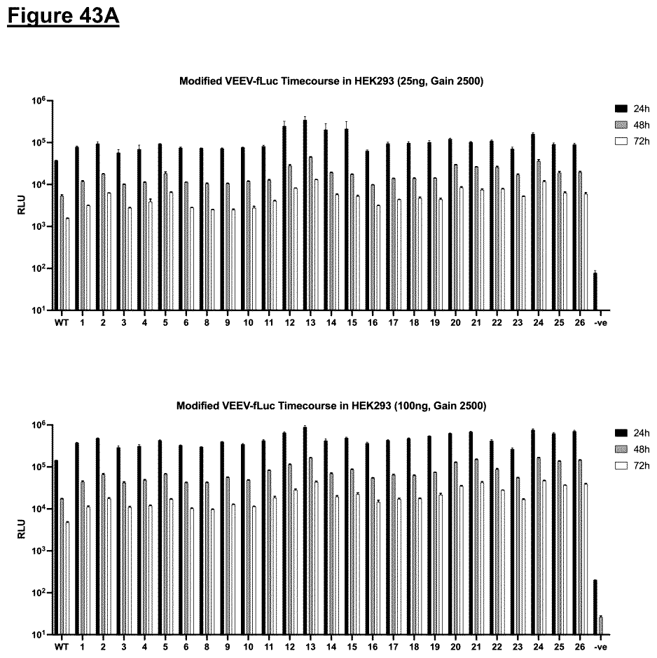

FIG. 42 shows the impact of individual and combined 2′-O-methyl NTP substitutions on capped mRNA expression of fLuc in THP-1 cells. Capped mRNA encoding fLuc was generated by IVT, where nucleotides were substituted with ≥75% 2′-O-methyl GTP, ≥75% 2′-O-methyl ATP, 90% 2′-O-methyl CTP or 90% 2′-O-methyl UTP alone or in different combinations. Substitution of all four NTPs is indicated by 2′-O-methyl combo. THP-1 cells were transfected with 25 ng of capped mRNA formulated in lipofectamine. Luciferase expression was monitored over 48 h by measurement of relative light units (see methods).

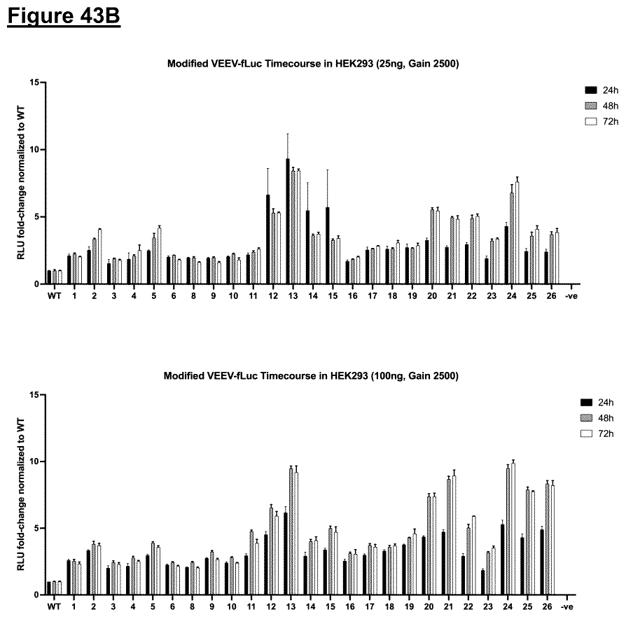

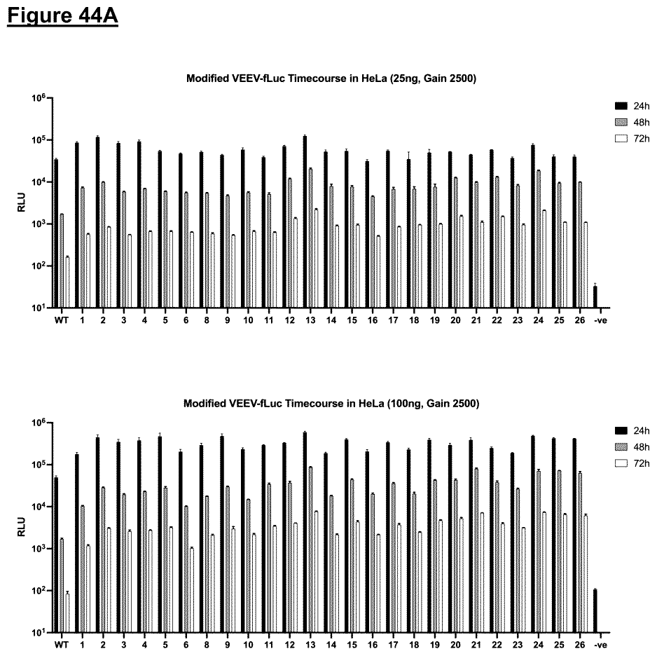

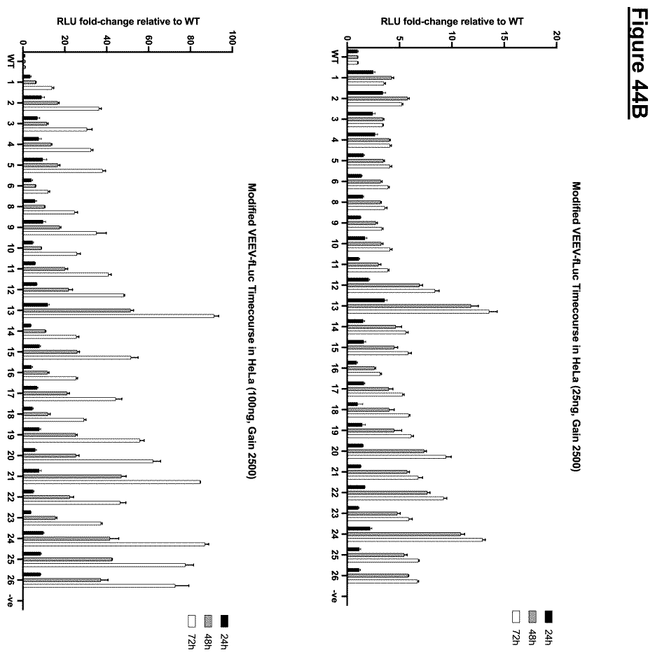

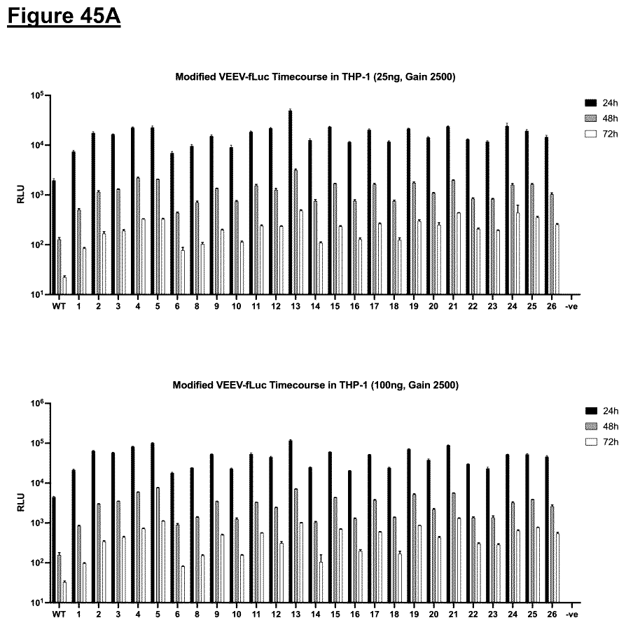

FIG. 43 shows the impact of combined 2′-O-methyl NTP substitutions on capped mRNA expression of fLuc in HEK293T cells. Conditions 1-26 are detailed in Table 6. Capped mRNA encoding fLuc was generated by IVT, where nucleotides were substituted according to Table 6. HEK293T cells were transfected with 25 ng and 100 ng of capped mRNA formulated in lipofectamine. A) Luciferase expression was monitored over 72 h by measurement of relative light units (see methods). B) Fold change in expression relative to unmodified mRNA.

FIG. 44 shows the impact of combined 2′-O-methyl NTP substitutions on capped mRNA expression of fLuc in Hela cells. Conditions 1-26 are detailed in Table 6. Capped mRNA encoding fLuc was generated by IVT, where nucleotides were substituted according to Table 6. HeLa cells were transfected with 25 ng and 100 ng of capped mRNA formulated in lipofectamine. A) Luciferase expression was monitored over 72 h by measurement of relative light units (see methods). B) Fold change in expression relative to unmodified mRNA.

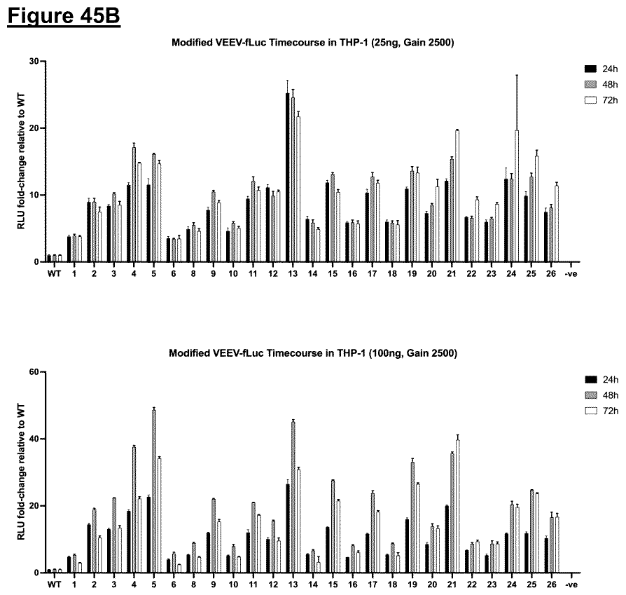

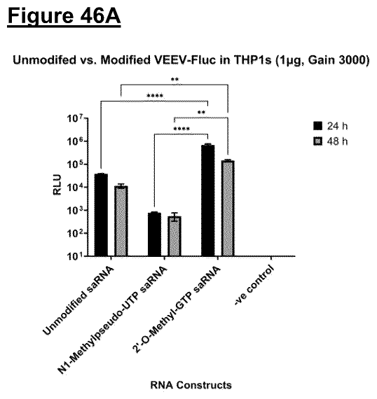

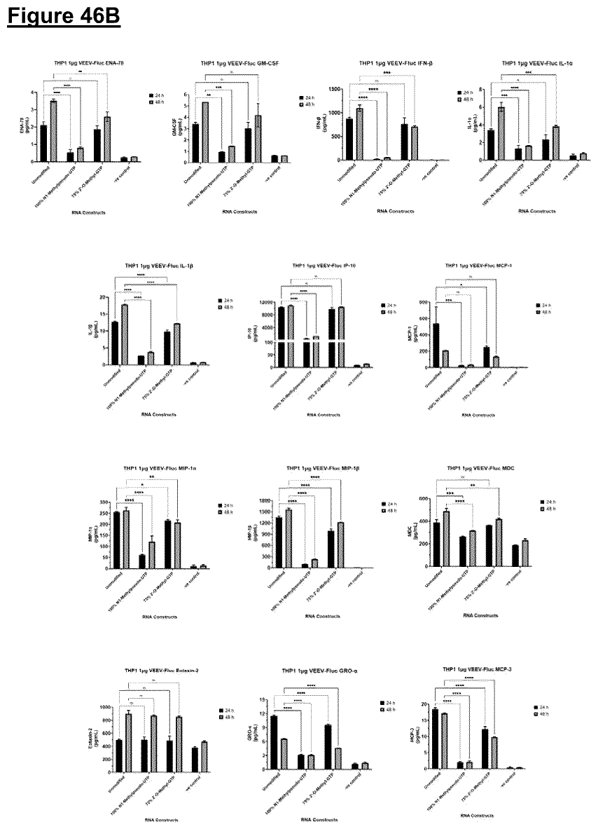

FIG. 45 shows the impact of combined 2′-O-methyl NTP substitutions on capped mRNA expression of fLuc in THp-1 cells. Conditions 1-26 are detailed in Table 6. Capped mRNA encoding fLuc was generated by IVT, where nucleotides were substituted according to Table 6. THP-1 cells were transfected with 25 ng and 100 ng of capped mRNA formulated in lipofectamine. A) Luciferase expression was monitored over 72 h by measurement of relative light units (see methods). B) Fold change in expression relative to unmodified mRNA.

FIG. 46 shows the impact of 2′-O-methyl GTP substitution on the inflammatory profile of saRNA in THP-1 cells. Capped saRNA encoding firefly luciferase was generated by IVT, using unmodified NTPs, where GTP was substituted with ≥75% 2′-O-methyl modified NTP, and where UTP was replaced with 100% N-1 methylpseudouridine. Unmodified and modified saRNA were transfected in 24-well plates of THP1 cells at 1 μg in triplicates (Error bars represent n=3). (A) At 24 and 48 h post transfection, half of the cell supernatant were collected before luciferase assay was performed to measure antigen expression. (B) Cell supernatants from each timepoint were then used to run an MSD assay to determine the different levels of cytokines stimulated by each RNA construct.

FIG. 47 shows the impact of 2′-O-methyl substitution on capped mRNA expression of eGFP. Capped mRNA encoding eGFP was generated by IVT, using unmodified NTPs, where UTP was replaced with 100% N-1 methylpseudouridine or where NTPs were substituted by 2′-O-methyl modified NTPs (75% Gm, 75% Am, 90% Cm, 90% Um) and labelled as 2′-O-methyl combo. Unmodified and modified mRNA were transfected in 24-well plates of (A) HELA and (B) THP1 cells at 1 μg in triplicates (Error bars represent n=3). 24 h post transfection, cells were harvested and stained before assessing for eGFP expression through flow cytometry. Data show the % of cells expressing eGFP (left vertical axis) and the Medium Fluorescent Intensity (right vertical axis).

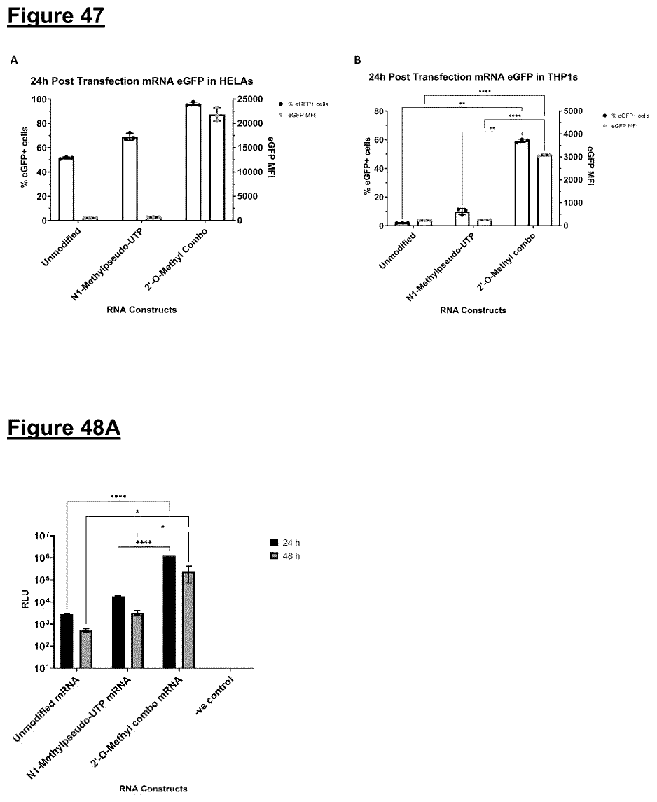

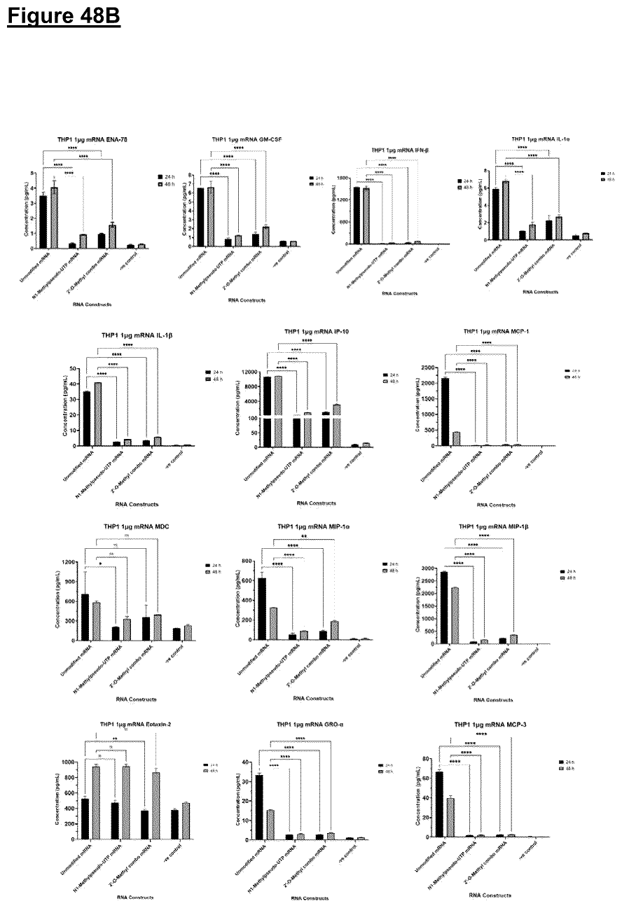

FIG. 48 shows the impact of 2′-O-methyl substitution on the inflammatory profile of mRNA in THP-1 cells. Capped mRNA encoding firefly luciferase was generated by IVT, using unmodified NTPs, where UTP was substituted for N1-Methylpseudouridine or where WT NTPs were substituted by 2′-O-methyl modified NTPs (75% Gm, 75% Am, 90% Cm, 90% Um) and labelled as 2′-O-methyl combo. Unmodified and modified mRNA were transfected in 24-well plates of THP1 cells at 1 μg in triplicates (Error bars represent n=3). (A) At 24 and 48 h post transfection, half of the cell supernatant were collected before luciferase assay was performed to measure antigen expression. (B) Cell supernatants from each timepoint were then used to run an MSD assay to determine the different levels of cytokines stimulated by each RNA construct.

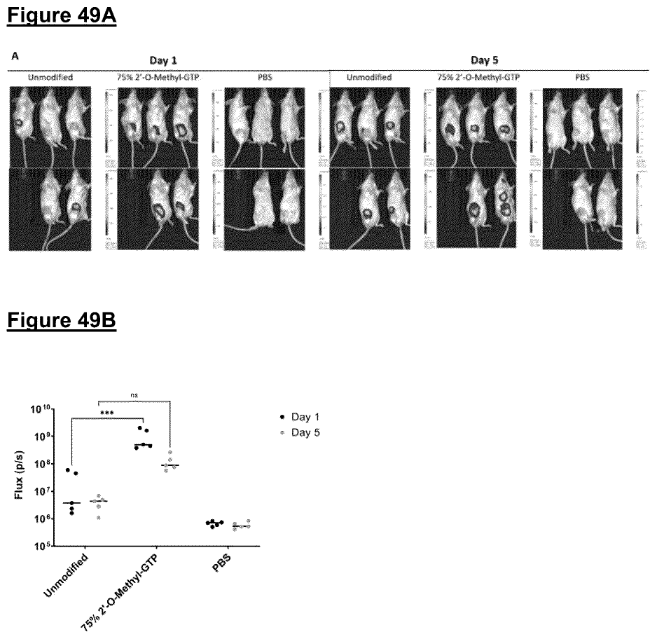

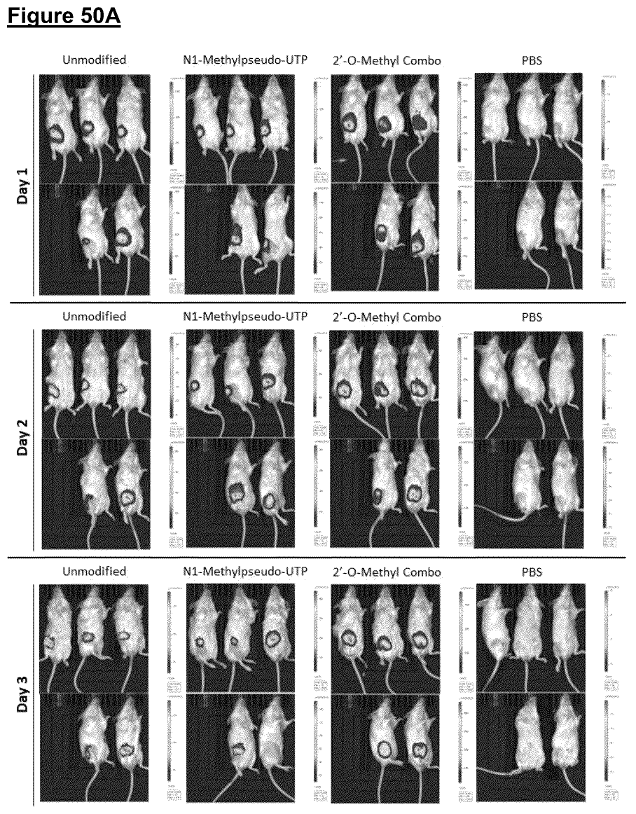

FIG. 49 shows the impact of 2′-O-methyl GTP substitution on saRNA expression in vivo. Capped saRNA encoding firefly luciferase was generated by IVT, using unmodified NTPs and where GTP was substituted with ≥75% 2′-O-methyl modified NTP. saRNA was formulated in a lipid nanoparticle composed of C12-200 ionizable lipid with DSPC, cholesterol and DMPE-PEG200. Groups of five female balb/c mice (n=5 per group) were given an I.M. administration of 10 μg LNP formulated unmodified or modified VEEV-fLuc saRNA. (A) Mice were imaged at day 1 and 5 using an IVIS Spectrum In Vivo Imaging System. (B) The luciferase expression was quantified as photons per second using an Aura Imaging Software. P=0.0005.

FIG. 50 shows the impact of 2′-O-methyl GTP substitution on mRNA expression in vivo. Capped mRNA encoding firefly luciferase was generated by IVT, using unmodified NTPs, where UTP was substituted for N1-Methylpseudouridine or where WT NTPs were substituted by 2′-O-methyl modified NTPs (75% Gm, 75% Am, 90% Cm, 90% Um) and labelled as 2′-O-methyl combo. mRNA was formulated in a lipid nanoparticle composed of C12-200 ionizable lipid with DSPC, cholesterol and DMPE-PEG200. Groups of five female balb/c mice (n=5 per group) were given an I.M. administration of 8 μg LNP formulated unmodified or modified mRNA. (A) Mice were imaged on days 1-3 using an IVIS Spectrum In Vivo Imaging System. (B) The luciferase expression was quantified as photons per second using an Aura Imaging Software. P<0.001.

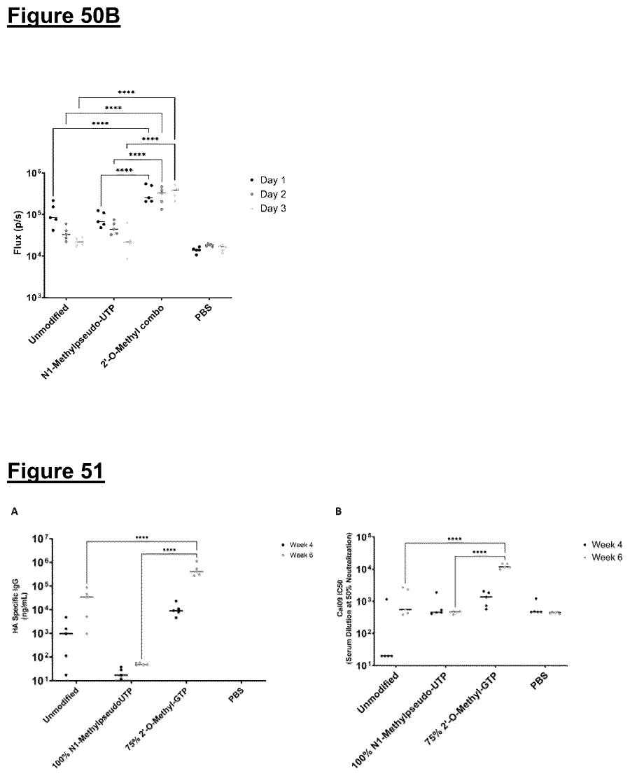

FIG. 51 shows that 2′-O-Methyl-GTP modified saRNA displays enhanced immunogenicity in vivo. VEEV saRNA encoding the hemagglutinin protein of influenza (VEEV-Ha) downstream of the sub-genomic promotor was generated by IVT: using unmodified NTPs; where GTP was substituted with ≥75% 2′-O-methyl modified NTP; or where UTP was replaced with 100% N-1 methylpseudouridine. Four groups of female balb/c mice (n=5 per group) were given a prime-boost regimen with an I.M. administration of 10 μg LNP formulated unmodified or modified VEEV-HA saRNA, where mice were bled and immunized at week o and 4, before being bled again at week 6. (A) Total HA specific IgG were quantified from the mice sera collected at week 4 and 6 by ELISA and (B) neutralising antibody responses were quantified by neutralization assay with the Influenza A California 2009 H1N1 virus. **** Two-way anova-Tukey's multiple comparisons test P<0.0001.

### EXAMPLES

### Materials and Methods

### In Vitro Transcription (IVT)

saRNA and mRNA encoding fLuc was produced by in-vitro transcription (IVT) using a linearised DNA template. The final volume for each IVT reaction was 50 uL, and the standardised IVT reaction mix was as follows:

TABLE 1
Standardised IVT reaction mix

0.5 μg DNA template
40 mM DTT
40 mM HEPES
4 U RNase Inhibitor per 1 μg DNA
100 U T7 RNA polymerase per 1 μg DNA
75 mM Mg(OAc)2
10 mM Na(OAc)
10 mM of each GTP, ATP, CTP, UTP
0.2 mM Spermidine

Where indicated, the concentration of magnesium acetate was varied (Mg(OAc)₂) or replaced by magnesium chloride, or manganese acetate. The total concentration of each NTP was kept at 10 nM, however, the relative proportion of unmodified to modified NTP was varied as indicated. For example, where it is described that 75% GTP was substituted with 2′ O-methyl-GTP, it indicates that the reaction contained 7.5 mM 2′-O-Methyl-GTP plus 2.5 mM GTP. IVT reactions were performed at 37° C. for 4 h. RNA yield was then measured right after IVT using the Qubit RNA Broad Range Assay kit with the Qubit Fluorometer (Thermo Fisher, UK) according to the manufacturer's protocol. Prior to post capping, RNA was purified using Lithium Chloride precipitation. Post capping of RNA was performed using ScriptCap™ Cap 1 Capping System (CellScript, UK) and reaction was incubated at 37° C. for 2 h. After post capping, RNA was purified again using lithium chloride precipitation and the final RNA concentration was measured using Nanodrop One (Thermo Scientific, UK). In order to assess the quality of the RNA, purified RNAs and the RNA Millennium Marker Ladder (Thermo Fisher, UK) were mixed with 2× NorthernMax-Gly Sample Loading Dye (Thermo Fisher, UK) and incubated at 50° C. for 30 min to denature the RNA. A 1.2% agarose gel with 1× NorthernMax Running Buffer (Thermo Fisher, UK) was prepared. After incubation, the denatured ladder and samples were loaded on to the gel and the gel was ran at 80 V for 45 min. The gel was then imaged on a GelDoc-It2 (UVP, UK).

### Cells and In Vitro Transfections

HEK293T.17 and HeLa cells (ATCC, USA) were cultured in complete Dulbecco's Modified Eagle's Medium (DMEM) (Gibco, Thermo Fisher, UK) containing 10% fetal bovine serum (FBS), 1% L-glutamine and 1% penicillin-streptomycin (Thermo Fisher, UK). THP-1 cells (ATCC, USA) were cultured in complete Roswell Park Memorial Institute (RPMI) 1640 (Gibco, Thermo Fisher, UK) containing 10% fetal bovine serum (FBS), 1% L-glutamine and 1% penicillin-streptomycin (Thermo Fisher, UK). Cells were plated in a 96-well plate 24 h prior to transfection at a density of 7×10⁴ cells per well for HEK293T.17 and HeLa cells; and 1×10⁵ cells per well THP-1 cells. Transfection of saRNA and mRNA encoding fLuc was performed using Lipofectamine MessengerMAX (Thermo Fisher, UK) according to the manufacturer's instructions. Firefly luciferase was measured ONE-Glo Luciferase Assay System (Promega, UK) according to the manufacturer's instructions and the luminescence signal was measured using FLUOstar Omega Plate Reader (BMG Labtech, UK).

### Example 1—the Incorporation of Modified Base 2′Methyl-NTPs (2-OMe-NTP) can be Achieved when Using Magnesium Ion Concentrations of Greater than 20 mM

The inventors have surprisingly demonstrated that incorporation of modified base 2′methyl-NTPs (2-OMe-NTP) can be achieved when using magnesium ion concentrations that are greater than 20 mM (see FIG. 1). In particular, the inventors have observed that magnesium acetate concentrations of greater than 20 mM are required for efficient incorporation of 2′methyl-GTPs, and ideally concentrations of ≤75 mM. However, the inventors believe that the maximum concentration of magnesium acetate could even be greater than 80 mM.

All experiments were performed with wild type Ty polymerase, however, the inventors expect that the reaction conditions described herein would also be advantageous for mutated versions to T7 polymerase shown to be more permissive for base substitutions, and also other RNA polymerases including T7, T3 and SP6.

### Example 2—Barely Detectable Incorporation Using Magnesium Chloride

Surprisingly, the inventors found that barely detectable incorporation was observed when using magnesium chloride, which is most commonly used for in vitro transcription reactions (see FIG. 2).

### Example 3—Manganese Acetate was Ineffective at Incorporating 2′Methyl-Ribose Modified GTP

Furthermore, replacement of magnesium with a different ion, manganese in the form of manganese acetate was also ineffective at incorporating 2′methyl-ribose modified GTP (FIG. 3).

### Example 4—Incorporation of 2′Methyl-GTP Increases Both the Magnitude and Expression of Uncapped saRNA in Cells that have Impaired Innate Sensing Mechanisms for Recognizing RNA (e.g. HEK293T Cells)

As described in the following examples, the inventors assessed the impact of incorporation of 2′methyl-ribose modified NTPs on the function of saRNA in three cell lines: Human embryonic kidney 293T (HEK293T) cells that have impaired innate sensing mechanisms that may lead to suppressed RNA expression, HeLa cells representative of epithelial cells and THP-1 cells representative of myeloid cells.

To determine whether potential advantages of incorporating 2′methyl-ribose modified NTPs were influenced by 5′ capping, the inventors assessed the impact on both uncapped and capped saRNA and mRNA. The saRNA construct used in these experiments encodes the sequence for Firefly luciferase (fLuc) downstream of the subgenomic promotor and expression of luciferase is detected by luciferase activity in cells overtime.

In transfection experiments performed in HEK293T cells when using 25 ug of uncapped saRNA formulated with lipofectamine, the inventors observed that incorporation of 2′methyl-GTP increased luciferase expression over 72 hs (FIG. 4) with a fold increase of ranging from 2-fold with 25% GM substitutions to 6-fold with 75% substitution (FIG. 4b). A similar trend was observed when using 100 ng of uncapped saRNA (FIGS. 5a & b). These data indicate that incorporation of 2′methyl-GTP (>25%) increases both the magnitude and expression of uncapped saRNA in cells that have impaired innate sensing mechanisms for recognizing RNA. This suggests that saRNA modified with 2′methyl-GTP may provide enhanced expression and stability even in cells with impaired RNA sensing mechanisms, and that such enhancement is not dependent upon RNA having a 5′ Cap.

### Example 5—Incorporating 2′Methyl-Ribose Modified NTPs Enhances the Magnitude and Duration of Capped saRNA Expression in Cells that have Impaired Innate RNA Sensing (e.g. HEK293T Cells)

The inventors then determined the impact of incorporation 2′methyl-ribose modified NTPs on the function of 5′ capped saRNA in HEK293T cells. In transfection experiments performed in HEK293T cells when using 25 ug of capped saRNA formulated with lipofectamine, the inventors observed that expression of unmodified saRNA was increased by one log relative to uncapped RNA (FIG. 6a). Nevertheless, when cells were transfected with 25 ng of saRNA, increased levels of expression over 72 h were observed when GTP was substituted with 2′-O-methyl GTP at a ratio ≥25% (FIG. 6b), with some modest increases in expression also seen for 2′-O-methyl ATP, CTP and UTP substitutions. A similar trend was observed with using 100 ng saRNA for transfection, with the greatest effect observed for substitution of GTP with 2′-O-methyl GTP (FIGS. 7a & 7b). These data indicate that incorporation 2′methyl-ribose modified NTPs and in particular Gm enhances the magnitude and duration of saRNA expression in cells that have impaired innate RNA sensing that may suppress RNA translation and increase degradation. The inventors anticipate that further gains may be realized when 2′-O-methyl GTP is used in combinations with 2′-O-methyl ATP, CTP and UTP substitutions.

For example, for saRNA these are:
1. 75% Gm
2, 75% Gm plus 90% Am and 90% Um
3. 75% Gm plus 75% Cm (with or without 90% Am+Um)
4. 75% Gm plus 50% Cm (with or without 90% Am+Um)
5. 50% Gm plus 50% Cm (with or without 90% Am+Um)

However, there are likely to be other iterations that may work, such as:
1. 50% Gm+30% Cm+50% Am+30% Um;
2. 50% Gm+90% Cm+50% Am+30% Um;
3. 50% Gm+30% Cm+50% Am+90% Um;
4. 50% Gm+90% Cm+50% Am+90% Um;
5. 25% Gm+30% Cm+25% Am+60% Um;
6. 25% Gm+90% Cm+50% Am+60% Um;
7. 75% Gm+30% Cm+50% Am+60% Um;
8. 75% Gm+60% Cm+50% Am+75% Um;
9. 75% Gm+90% Cm+50% Am+60% Um;
10. 50% Gm+30% Cm+25% Am+60% Um;
11. 50% Gm+90% Cm+25% Am+60% Um;
12. 50% Gm+30% Cm+75% Am+60% Um;
13. 50% Gm+90% Cm+75% Am+60% Um;
14. 25% Gm+60% Cm+50% Am+30% Um;
15. 25% Gm+60% Cm+50% Am+90% Um;
16. 75% Gm+60% Cm+50% Am+30% Um;
17. 75% Gm+60% Cm+50% Am+90% Um;
18. 50% Gm+60% Cm+25% Am+30% Um;
19. 50% Gm+60% Cm+25% Am+90% Um;
20. 50% Gm+60% Cm+75% Am+30% Um;
21. 50% Gm+60% Cm+75% Am+90% Um;
22. 25% Gm+60% Cm+25% Am+60% Um;
23. 75% Gm+60% Cm+25% Am+60% Um;
24. 25% Gm+60% Cm+75% Am+60% Um;
25. 75% Gm+60% Cm+75% Am+60% Um;
26. 50% Gm+60% Cm+50% Am+60% Um;
27. 50% Gm+60% Cm+75% Am+75% Um;
28. 50% Gm+60% Cm+60% Am+60% Um;
29. 50% Gm+50% Cm+50% Am+50% Um; or any other percentage ratio

For mRNA, certain combinations would include:
1. 75% Gm+90% Cm (with or without 90% Am+Um)
2. 75% Gm+75% Cm (with or without 90% Am+Um)
3. 50% Gm+50% Cm (with or without 90% Am+Um)
4. 25% Gm+25% Cm (with or without 90% Am+Um)

However, as with saRNA, there are likely to be other iterations of mRNA that could work, such as:
1. 50% Gm+30% Cm+50% Am+30% Um;
2. 50% Gm+90% Cm+50% Am+30% Um;
3. 50% Gm+30% Cm+50% Am+90% Um;
4. 50% Gm+90% Cm+50% Am+90% Um;
5. 25% Gm+30% Cm+25% Am+60% Um;
6. 25% Gm+90% Cm+50% Am+60% Um;
7. 75% Gm+30% Cm+50% Am+60% Um;
8. 75% Gm+60% Cm+50% Am+75% Um;
9. 75% Gm+90% Cm+50% Am+60% Um;
10. 50% Gm+30% Cm+25% Am+60% Um;
11. 50% Gm+90% Cm+25% Am+60% Um;
12. 50% Gm+30% Cm+75% Am+60% Um;
13. 50% Gm+90% Cm+75% Am+60% Um;
14. 25% Gm+60% Cm+50% Am+30% Um;
15. 25% Gm+60% Cm+50% Am+90% Um;
16. 75% Gm+60% Cm+50% Am+30% Um;
17. 75% Gm+60% Cm+50% Am+90% Um;
18. 50% Gm+60% Cm+25% Am+30% Um;
19. 50% Gm+60% Cm+25% Am+90% Um;
20. 50% Gm+60% Cm+75% Am+30% Um;
21. 50% Gm+60% Cm+75% Am+90% Um;
22. 25% Gm+60% Cm+25% Am+60% Um;
23. 75% Gm+60% Cm+25% Am+60% Um;
24. 25% Gm+60% Cm+75% Am+60% Um;
25. 75% Gm+60% Cm+75% Am+60% Um;
26. 50% Gm+60% Cm+50% Am+60% Um;
27. 50% Gm+60% Cm+75% Am+75% Um;
28. 50% Gm+60% Cm+60% Am+60% Um;
29. 50% Gm+50% Cm+50% Am+50% Um; or any other percentage ratio.

### Example 6—Incorporation of 2′Methyl-Ribose Modified NTPs Increases the Magnitude and Expression of Uncapped saRNA in Cells that have Intact Innate Sensing Mechanisms for Recognizing RNA (e.g. HeLa Cells)

Next, the inventors assessed the impact of incorporation of 2′methyl-ribose modified NTPs on the function of saRNA in Hela cells representative of epithelial cells. Unlike the HEK293T cells tested in the previous example, HeLa cells have intact epithelial innate sensing mechanisms able to respond to RNA triggering intracellular signaling pathways able to reduce RNA expression and enhance the rate of RNA degradation [6-8].

First, the inventors looked at the impact of incorporating 2′-O-methyl modified bases into uncapped saRNA. When using 25 ng saRNA for transfection, the inventors observed increase in expression and duration of fLuc expression over 72 h for 2′-O-methyl GTP (FIG. 8a), with fold increases of between 10- to 25-fold when using a ratio of 75% 2′-O-methyl GTP to 25% unmodified GTP (FIG. 8b). Modest increases in expression were observed for both 2′-O-methyl ATP and UTP substations at 48 h. When using 100 ng for transfection, however, the inventors observed increased expression when using 2′-O-methyl GTP substitution (FIG. 9a) at 72 h demonstrating up to 50-fold increase (FIG. 9b). Surprisingly, the inventors observed increased expression with 2′-methyl-ATP or CTP substitution at 72 h, and for 2′-methyl-ATP this demonstrated up to 100-fold increase in expression. These data indicate that different substitutions may have different impact on the kinetics of expression.

### Example 7—Incorporation of 2′Methyl-Ribose Modified NTPs Increases the Magnitude and Expression of Capped saRNA in Cells that have Intact Innate Sensing Mechanisms for Recognizing RNA (e.g. HeLa Cells)

The inventors then assessed the impact of incorporation 2′methyl-ribose modified NTPs on capped RNA. Expression of non-modified 5′ capped saRNA (25 ng) was one log higher in Hela cells than that seen for uncapped saRNA (FIG. 10a). Increased expression relative to unmodified saRNA was observed for 2′O-methyl GTP substitution, although the fold change was less pronounced to that seen with uncapped RNA (FIG. 10b). Modest, but detectable, changes were still observed for substitutions with 2′O-methyl ATP, CTP and UTP. A similar trend was observed when using 100 ng for transfection (FIG. 11a). Here again, 2′O-methyl GTP substitution displayed the strongest impact on the magnitude and duration of expression with up 20-fold increase observed at 48 h (FIG. 11b). Smaller, but detectable changes, were also seen for 2′-methyl-ATP or CTP substitutions.

These data indicate that substation of individual nucleotides and in particular, GTP with 2′O-methyl base modification, enhances saRNA expression. It is anticipated that the use of 2′O-methyl-GTP in combinations with 2′O-methyl ATP, CTP and GTP also lead to further gains in expression.

### Example 8—Incorporation of 2′Methyl-Ribose Modified NTPs Increases the Magnitude and Expression of Uncapped saRNA in Cells that have Sensitive Innate Sensing Mechanisms for Recognizing RNA (e.g. THP-1 Cells)

The inventors then assessed the impact of incorporation 2′methyl-ribose modified NTPs on the function of saRNA in THP-1 cells representative of myeloid cells, specifically monocytic cells. These cells are particularly resistant to transfection due to sensitive innate sensing mechanisms able to respond to RNA triggering intracellular signaling pathways able to reduce RNA expression and enhance the rate of RNA degradation.

The inventors first looked at the impact of incorporating 2′-O-methyl modified bases into uncapped saRNA. When using 25 ng saRNA for transfection, they observed a dramatic increase in fLuc expression for 2′-O-methyl GTP (FIG. 12a), with fold increase of greater than 1000-fold when using a ratio of 75% 2′-O-methyl GTP to 25% unmodified GTP at 48 h (FIG. 12b) and detectable expression at 72 h, where none was observed for unmodified saRNA. Some improvement in expression was observed for 2′ -O-methyl ATP, and UTP substitutions. When using 100 ng for transfection, the inventors again observed increased expression when using 2′-O-methyl GTP substitution (FIG. 13a) at 72 h demonstrating up to 80-fold increase (FIG. 13b). Substitutions with 2′-O-methyl ATP, CTP and UTP had less impact on expression than 2′-O-methyl GTP.

### Example 9—Incorporation of 2′Methyl-Ribose Modified NTPs Increases the Magnitude and Expression of Capped saRNA in Cells that have Sensitive Innate Sensing Mechanisms for Recognizing RNA (e.g. THP-1 Cells)

Subsequently, the inventors assessed the impact of incorporation of 2′methyl-ribose modified NTPs on capped RNA in THP-1 cells. Expression of non-modified 5′ capped saRNA (25 ng) was again one log higher in THP-1 cells than that seen for uncapped saRNA (FIG. 14a). Increased expression relative to unmodified saRNA was observed for 2′O-methyl GTP substitution, with fold change ranging from 5-10-fold over 72 h (FIG. 14b), however, this was less pronounced to that seen with uncapped RNA. Modest, but detectable, changes were observed for substitutions with 2′O-methyl ATP, CTP and UTP. A similar trend for 2′O-methyl GTP substitution was observed when using 100 ng for transfection (FIG. 15a). Here again 2′O-methyl GTP substitution displayed the strongest impact on the magnitude and duration of expression with up 40-fold increase observed at 72 h (FIG. 15b). There was little observable advantage discernable for substitutions with 2′O-methyl ATP, CTP and UTP.

These data indicate that substitution of GTP with 2′O-methyl base modification enhances saRNA expression in myeloid cells. It is possible that the use of 2′O-methyl-GTP in combinations with 2′O-methyl ATP, CTP and GTP may lead to further gains in expression.

### Example 10—N1 Methyl-Pseudo-UTP Provides No Advantage to saRNA Expression

Since N1 methyl-pseudouridine had been previously shown to increase expression of mRNA, the inventors determined whether this provided any advantage to the expression of saRNA. Here, the inventors looked at substitution of UTP with N1 methyl-pseudo-UTP from 25-100%. Transfection experiments were performed with 100 ng of uncapped or capped 100 ng saRNA formulated with lipofectamine. The inventors observed no apparent advantage with respect to fLuc expression in either HEK293T or HeLa cells (FIGS. 16A and B, respectively). These data therefore suggest that in contrast to 2′O-methyl GTP, N1 methyl-pseudo-UTP provides no advantage to saRNA expression.

### Example 11—2′O-Methyl GTP, 7-Deaza-GTP Substitution Increases the Magnitude and Duration of saRNA Expression

The inventors also assessed the impact of incorporating 7-deazaguanosine-5-triphosphate (7-deaza-GTP), which has been previously reported to reduce innate recognition of RNA by Toll Like receptor engagement [9]. Here, the inventors looked at substitution of GTP with 7-deaza-GTP from 25-100%. Transfection experiments were performed with 100 ng of uncapped or capped 100 ng saRNA formulated with lipofectamine. The inventors observed modest enhanced expression in HEK293T cells at 24, 48 and 72 h (FIG. 17A). They also observed enhanced expression in HeLa cells, particularly at 72 h (FIG. 17B) (2 fold) suggesting that 7-deaza-GTP substitution enhances the duration of expression in HeLa cells. These data suggest that similar to 2′O-methyl GTP, 7-deaza-GTP substitution increases the magnitude and duration of saRNA expression, although to a far lesser extent.

### Example 12—2′-Fluoro-Modified NTPs does not Affect saRNA Expression

As substitution of the ribose 2′ OH with a 2′ methyl group modulated saRNA expression, the inventors performed additional experiments to determine the impact of incorporating nucleotides with a smaller 2′ fluoro group, previously show to modulate innate immune activation [10]. Here, they looked at substitution of ATP, CTP, UTP and TTP with 2′-fluoro-modified NTPs from 25-100%. Transfection experiments were performed with 100 ng of uncapped or capped 100 ng saRNA formulated with lipofectamine. The inventors observed no change in expression of fLuc capped and uncapped saRNA in HEK293T cells (FIG. 18) or HeLa cells (FIG. 19) with different proportions of 2′ fluoro ATP, CTP, TTP or UTP Figure.

### Example 13—Summary of the Data

Tables 2 and 3 below summarise the impact of base modifications on saRNA expression.

TABLE 2
The impact of base modifications on saRNA expression
when using 25 ng saRNA with 75% substitution

| Base modification | HEK293T | HeLa | THP-1 |
|---|---|---|---|
| 2′ O methyl-GTP | + | ++ | ++ |
| 2′ O methyl-ATP | − | + | +/− |
| 2′ O methyl-CTP | − | + | + |
| 2′ O methyl-UTP | − | + | + |

TABLE 3
the impact of base modifications on saRNA
expression when using 100 ng saRNA

| Base modification | HEK293T | HeLa | THP-1 |
|---|---|---|---|
| 2′ O methyl-GTP | + | +++ | +++ |
| 2′ O methyl-ATP | − | +++ | +/− |
| 2′ O methyl-CTP | − | +++ | ++ |
| 2′ O methyl-UTP | − | ++ | + |
| N1 Me-pseudo-UTP | − | − | ND |
| 7-deaza-GTP | + | + | ND |
| 2′ Fluoro-ATP | − | − | ND |
| 2′ Fluoro-CTP | − | − | ND |
| 2′ Fluoro-TTP | − | − | ND |
| 2′ Fluoro-UTP | − | − | ND |

+ = 1-5 fold,
++ = 5-10 fold,
+++ = >10 fold,
ND = not determined.

### Example 14—Substitution of GTP with 2′ O-Methyl GTP Enhances the Expression of mRNA

Having determine that 2′ O-methyl NTP substitutions enhanced the expression of saRNA, the inventors then assessed its impact on expression of mRNA, also encoding firefly luciferase (fLuc). They compared the expression of unmodified mRNA to that where 75% of GTP was substituted with 2′ O-methyl GTP, and mRNA where 75% of each of GTP and ATP and 90% of CTP and UTP (Combo mRNA) were replaced with 2′ O-methyl counterparts.

They assessed the impact of 2′ O-methyl substitutions on fLuc expression in HEK293T, HeLa and THP-1 cells as before. Using 5′ capped saRNA and mRNA, the inventors observed that substitution of GTP with 2′ O-methyl GTP led to a modest increase in expression at 24 h. However, combined substitutions of all four NTPs (FIG. 20a) enhanced expression over 72 h by up to 10-fold (FIG. 20b). mRNA expression was approximately two-fold lower than that seen with saRNA that displayed a similar pattern of enhanced expression on substitution of 75% of GTP with 2′ O-methyl GTP as reported above.

The inventors then assessed the expression in interferon competent HeLa cells (FIG. 21a). saRNA expression was enhanced approximately 1 log on substitution of 75% of GTP with 2′ O-methyl GTP (FIG. 21b). Expression of mRNA was similarly enhanced by 75% of GTP with 2′ O-methyl GTP. Surprisingly, combined substitutions of all for NTPs with 2′ O-methyl modified NTPs increased expression of mRNA to equivalent levels to that seen for saRNA (FIG. 21a) with fold increases of ≥ to 100-fold over 72 h (FIG. 21b).

They observed a similar pattern in THP-1 cells representative of monocytic cells (FIG. 22a). As observed for HeLa cells, saRNA expression was enhanced approximately 1 log over 72 h on substitution of 75% of GTP with 2′ O-methyl GTP (FIG. 22b). When assessing mRNA, expression also enhanced expression by 5-fold over 72 h, however, combined substitutions of all for NTPs with 2′ O-methyl modified NTPs increased expression of mRNA to equivalent levels to that seen for saRNA (FIG. 22a) with fold increases of ≥ to 100-fold over 72 h (FIG. 22b).

These data surprisingly demonstrate that substitution of GTP with 2′ O-methyl GTP enhances the expression of both saRNA and mRNA. Further substitutions of additional NTPs appears to provide additional benefit to the expression of mRNA and it is anticipated that the same would be likely for saRNA.

TABLE 4
The impact of base modifications on mRNA
expression when using 100 ng mRNA

| Base modification | HEK293T | HeLa | THP-1 |
|---|---|---|---|
| 2′ O methyl-GTP | + | ++ | ++ |
| 2′ O methyl- | + | +++ | +++ |
| Combo | | | |

+ = 1-5 fold,
++ = 5-10 fold,
+++ = >10 fold,
ND = not determined.

### Example 15—Substitution of GTP and CTP with 2′ O-Methyl-GTP (Gm) and CTP Enhances the Expression of mRNA

Having determined that combined 2′ O-methyl of all four NTP substitutions enhanced the expression of mRNA (FIGS. 20-22), the inventors then assessed the impact on expression (fLuc) of Gm substitution in combination with each of the other three NTPs relative to the combined substitution of all four NTPs (mRNA combo). They compared the expression of unmodified mRNA to that where 75% of GTP was substituted with 2′ O-methyl GTP alone, 75% substitution of GTP (Gm) with 75% ATP (Am), 75% GTP (Gm) with 90% CTP (Cm), 75% GTP (Gm) with 90% UTP (Um), and 75% of GTP, 75% ATP, 90% of CTP and UTP (Combo mRNA) were replaced with 2′ O-methyl counterparts.

They assessed the impact of 2′ O-methyl substitutions on fLuc expression in HEK293T, HeLa and THP-1 cells as before. Using 5′ capped mRNA, the inventors observed that substitution of GTP and CTP with 2′ O-methyl GTP and 2′ O-methyl CTP provided a similar enhancement to that seen when all four NTPs were substituted by 2′ O-methyl NTPs. In HEK293T cells, deficient in innate interferon response pathways this led to up to 20 fold enhancement in expression at 24 hrs (FIGS. 23 and 24).

The inventors then assessed the expression in interferon competent HeLa cells (FIG. 25). Surprisingly, expression of mRNA was similarly enhanced by substitution of 75% of GTP and 90% CTP with 2′ O-methyl GTP and 2′ O-methyl CTP to that where all four NTPs were substituted by 2′ O-methyl NTPs (FIG. 26) with fold increases of ≥ to 70-fold over 72 h (FIG. 26).

They observed a similar pattern in THP-1 cells representative of monocytic cells (FIG. 27). As observed for HeLa cells, expression of mRNA was similarly enhanced by substitution of 75% of GTP and 90% CTP with 2′ O-methyl GTP and 2′ O-methyl CTP to that where all four NTPs were substituted by 2′ O-methyl NTPs (FIG. 28) with fold increases of ≥ to 1000-fold over 72 h (FIG. 28).

These data surprisingly demonstrate that substitution of GTP with 2′ O-methyl GTP together with CTP with 2′ O-methyl GTP and 2′ O-methyl CTP enhances the expression mRNA to a similar extent to that where all four NTPs were substituted by 2′ O-methyl NTPs. Surprisingly, the greatest increases in expression were seen in interferon competent cells (HeLa and THP-1 cells) relative to HEK293T cells that have impaired interferon responses and innate signalling pathways.

### Example 16—Substitution of Individual NTPs with 2′ O-Methyl-NTPs (Nm) Provides Little of No Benefit to Expression of mRNA in HEK293T and Hela Cells

Having determined that combined substitution of GTP and CTP with 2′ O-methyl-GTP (Gm)_and -CTP (Cm) or substitution of all four NTPs enhanced the expression of mRNA (FIGS. 23-28), the inventors then assessed the impact on expression (fLuc) of individual NTP substitutions. They compared the expression of unmodified mRNA to that where 75% of GTP was substituted with 2′ O-methyl GTP (Gm), 75% substitution of ATP with 2′ O-methyl ATP (Am), 90% substitution of CTP with 2′ O-methyl CTP (Cm), or 90% substitution of UTP with 2′ O-methyl UTP (Um).

They assessed the impact of 2′ O-methyl substitutions on fLuc expression in HEK293T, and HeLa cells as before. Using 5′ capped mRNA, the inventors observed that individual substitution of GTP, ATP, CTP or UTP with 2′ O-methyl-GTP (Gm),-ATP (Am),-CTP (Cm), or -UTP (Um) had little or no impact on mRNA expression as determined by measurement of fLuc expression in HEK293T cells when using 25 ng (FIG. 29) or 100 ng mRNA (FIG. 30). This was also observed in HeLa cells when using 25 ng (FIG. 31) or 100 ng of mRNA (FIG. 32).

These data surprisingly demonstrate that combined substitution of GTP with 2′ O-methyl GTP together with CTP with 2′ O-methyl GTP provides a dominant effect with respect to enhanced expression in both HEK293T and HeLa cells, where individual substitutions of either GTP or CTP with 2′ O-methyl-GTP or -CTP provide little or no benefit over un-modified mRNA.

### Example 17—Substitution of Individual CTPs with 2′ O-Methyl-CTPs (Cm) Enhances mRNA Expression in THP-1 Cells

Having assessed the impact of individual NTP substitutions on mRNA expression in HEK293T cells and HeLa cells, the inventors then assessed the impact on expression (fLuc) of individual NTP substitutions in THP-1 cells. These cells are monocytic cells, highly sensitive to innate activation that triggers robust RNA degradation. They compared the expression of unmodified mRNA to that where 75% of GTP was substituted with 2′ O-methyl GTP (Gm), 75% substitution of ATP with 2′ O-methyl ATP (Am), 90% substitution of CTP with 2′ O-methyl CTP (Cm), or 90% substitution of UTP with 2′ O-methyl UTP (Um).

They assessed the impact of 2′ O-methyl substitutions on fLuc expression in THP-1 cells as before. Using 5′ capped mRNA, the inventors observed that individual substitution of GTP, ATP, CTP or UTP with 2′ O-methyl-GTP (Gm),-ATP (Am),-CTP (Cm), or -UTP (Um) had differential impact on mRNA expression as determined by measurement of fLuc expression in THP1 cells when using 25 ng (FIG. 33) or 100 ng mRNA (FIG. 35). Substitution of CTP with 2′ O-methyl CTP (Cm) provided the greatest impact on expression showing up to 10-fold increase with 25 ng of mRNA (FIG. 34) and 20-fold increase with 100 ng of mRNA (FIG. 36) relative to un-modified mRNA. Substitution of GTP with 2′ O-methyl GTP (Gm) provided a modest increase in expression of up to 3-fold increase with 25 ng of mRNA (FIG. 34) and 5-fold increase with 100 ng of mRNA (FIG. 36) relative to un-modified mRNA. Substitution of ATP or UTP with 2′ O-methyl ATP (Am) or 2′ O-methyl UTP (Um) had little or no impact on expression.

These data surprisingly demonstrate that while individual substitution of CTP or GTP NTPs with 2′ O-methylated versions enhanced expression in THP-1 cells, the combined substitution of GTP with 2′ O-methyl GTP together with CTP with 2′ O-methyl GTP provides a synergistic improvement in expression which is more than additive predicted from individual substitutions.

### SUMMARY

The inventors have demonstrated that 2′-substituted ribose modified NTPs, in particular 2′-O-methyl modified NTPs, can be used to increase (i) expression and/or translation of an RNA molecule comprising the one or more modified NTP; (ii) enhancing the stability of an RNA molecule comprising the one or more modified NTP; and/or (iii) reducing the activation of innate sensing, interferon generation and/or degradation of an RNA molecule comprising the one or more modified NTP.

In summary:
1. The invention involves the use of 2′-modified RNA, specifically mRNA, and saRNA and non-coding RNA, with a significant percentage of nucleotides (≥25, up to 100%) being 2′-methyl-NTPs. The mRNA and saRNA sequence are of a sizeable length (>100 bp) and wild-type nucleotides are at least partially substituted by 2′-O-methyl-NTPs.
2. The modified RNAs are synthesised via in vitro transcription, and the reaction mixture optimally includes high concentrations of magnesium ions (i.e. >20 mM magnesium ions, ideally 75 nM) and high concentrations of nucleotides (i.e. >10 mM nucleotides).
3. The inventors have shown that magnesium ions presented in magnesium acetate rather than magnesium chloride results in enhanced expression and incorporation of the modified NTPs.
4. Targeted partial substitution of wild-type GTP, ATP, CTP and/or UTP with respective 2′-methyl-NTP led to enhanced expression and stability of modified mRNA or saRNA, by ≤100-fold, in interferon-responsive competent cells.

### Example 18—Substitution of Modified NTPs Other than 2-O-Methyl NTP Fail to Provide Any Benefit with Respect to saRNA Expression

A wider range of modified NTP have been screened (see FIG. 39) with respect to their ability to enhance saRNA expression in human cells in vitro, as demonstrated in FIGS. 16, 17, 18, 19 and 40. These failed to significantly enhance and, in many cases, impaired fLuc saRNA expression. Of note, a number of modified NTPs previously shown to enhance mRNA express provided no benefit or reduced expression e.g. pseudouridine, N1-methyl pseudouridine, 5-methylcytidine (m5C), N6-methyladenosine (m6A), and 2-thiouridine (s2U) etc [30, 31].

These data indicate that saRNA requires specific modifications for enhanced expression relative to mRNA, and the data are summarised in Table 5 below.

TABLE 5
The impact of base modifications on saRNA expression

| | Abbrevi- | | | |
|---|---|---|---|---|
| NTP modification | ation | HEK293T | HeLa | THP-1 |
| N1 methyladenosine | m1A | None | None | None |
| N6-methyladenosine | m6A | Negative | Negative | Negative |
| 2′-Fluoro-2′-deoxy- | 2′-F-dA | None | None | None |
| adenosine | | | | |
| 5-methylcytidine | m5C | None | None | None |
| N4-methylcytidine | m4C | None | None | None |
| N4-acetylcytidine | ac4C | Negative | Negative | Negative |
| 5-Methoxycytidine | 5moC | None | None | None |
| 2′-Fluoro-2′-deoxy- | 2′-F-dC | None | None | None |
| cytidine | | | | |
| 7-deazaguanosine | 7-Deaza-G | + | + | ND |
| 2′-Fluoro-2′-deoxy- | 2′-F-dG | None | None | None |
| guanosine | | | | |
| Pseudouridine | ψ | Negative | Negative | Negative |
| N1-methylpseudouridine | m1ψ | Negative | Negative | Negative |
| 2-thiouridine | s2U | Negative | Negative | Negative |
| 5-methoxyuridine | 5moU | None | None | None |
| 2′-Fluoro-2′-deoxy- | 2′-F-dU | None | None | None |
| uridine | | | | |

### Example 19—2′-O-Methyl-GTP Enhances Expression of saRNA Encoding eGFP

Having determined that substitution of GTP with 2′-O-Methyl-GTP alone provided a major impact on fLuc expression, the inventors determined whether the same effect was observed if the GOI encoded in the saRNA was changed. VEEV saRNA encoding eGFP downstream of the sub-genomic promotor was generated by IVT: using unmodified NTPs; where GTP was substituted with ≥75% 2′-O-methyl modified NTP; and where UTP was replaced with 100% N-1 methylpseudouridine. These constructs were used to transfect HELA and THP1 cells, and at 24 h, cells were harvested, stained and eGFP expression assessed by flow cytometry. 2′-O-Methyl-GTP modified VEEV-eGFP saRNA elicits a higher number of eGFP positive cells and increased eGFP median florescent intensity (MFI) compared to unmodified and N1-Methypseudo-UTP modified VEEV-eGFP in both HELA (see FIG. 41A) and THP1 (see FIG. 41B) cells. These data indicate that the beneficial effects of 2′-O-Methyl-GTP are applicable to different encoded GOIs. Importantly, N1-Methylpseudo-UTP modified VEEV-eGFP did not produce any eGFP positive cells in both cell lines in contrast to published findings for conventional mRNA where inclusion of N1-Methylpseudo-UTP provides enhanced expression. These data highlight the surprising observation that different modifications are required for beneficial modifications of saRNA relative to mRNA.

### Example 20—Combined 2′-O-Methyl-NTP Substitutions Enhance Expression of mRNA Encoding fLuc

Having determined that substitution of GTP with 2′-O-Methyl-GTP alone provided a major impact on fLuc expression, the inventors determined whether further gains in expression could be delivered by combined 2′-O-Methyl-NTP substitutions. FLuc mRNA was generated by IVT: using unmodified NTPs; 100% N-1 methylpseudouridine substitution and where nucleotides were substituted with ≥75% 2′-O-methyl GTP, ≥75% 2′-O-methyl ATP, 90% 2′-O-methyl CTP or 90% 2′-O-methyl UTP alone or in different combinations. These constructs were used to transfect THP1 cells, and Luciferase expression was monitored over 48 h by measurement of relative light units. Combined substitution with 2′-O-methyl ATP plus UTP or 2′methyl GTP plus CTP provided enhanced expression relative to substitution of individual bases alone. However, combined substitution of all for NTPs with 2-O-methyl version provided the greatest enhancement.

### Example 21—Combined 2′-O-Methyl-NTP Substitutions Enhance Expression of mRNA Encoding fLuc

Having determined that combined substitution of 2′-O-Methyl-NTPs at a fixed ratio (2′-O-methyl combo (see FIG. 42) a major impact on fLuc expression, the inventors determined how the ratio of 2-Omethy-NTP substitution using all four NTPs influenced expression. FLuc mRNA was generated by IVT: using unmodified NTPs and a wide range of different 2-Omethy-NTP substitutions as detailed in Table 6 below.

TABLE 6
Percentage substitutions used for 2-O-methyl modified
NTPs. Condition 1-26 are evaluated in FIGS. 43-45

| ID | Cm | Um | Gm | Am |
|---|---|---|---|---|
| 1 | 30 | 30 | 50 | 50 |
| 2 | 90 | 30 | 50 | 50 |
| 3 | 30 | 90 | 50 | 50 |
| 4 | 90 | 90 | 50 | 50 |
| 5 | 90 | 90 | 50 | 50 |
| 6 | 30 | 60 | 25 | 50 |
| 7 | 90 | 60 | 25 | 50 |
| 8 | 30 | 60 | 75 | 50 |
| 9 | 90 | 60 | 75 | 50 |
| 10 | 30 | 60 | 50 | 25 |
| 11 | 90 | 60 | 50 | 25 |
| 12 | 30 | 60 | 50 | 75 |
| 13 | 90 | 60 | 50 | 75 |
| 14 | 60 | 30 | 25 | 50 |
| 15 | 60 | 90 | 25 | 50 |
| 16 | 60 | 30 | 75 | 50 |
| 17 | 60 | 90 | 75 | 50 |
| 18 | 60 | 30 | 50 | 25 |
| 19 | 60 | 90 | 50 | 25 |
| 20 | 60 | 30 | 50 | 75 |
| 21 | 60 | 90 | 50 | 75 |
| 22 | 60 | 60 | 25 | 25 |
| 23 | 60 | 60 | 75 | 25 |
| 24 | 60 | 60 | 25 | 75 |
| 25 | 60 | 60 | 75 | 75 |
| 26 | 60 | 60 | 50 | 50 |

These constructs were used to transfect HEK293T, HeLa and THP1 cells, and Luciferase expression was monitored over 72 h by measurement of relative light units (see FIGS. 43-45). Combined substitution with all four 2′-O-methyl NTPs across a wide range of ratios provided enhanced expression relative to unmodified mRNA. These data indicate that substitution of all four NTPs with 2-O-methyl NTPs across a wide range of substitution ratios is beneficial for enhanced mRNA expression.

### Example 22—2′-O-Methyl-GTP Modified saRNA is Less Inflammatory than Unmodified saRNA

To determine whether the 2′-O-Methyl modification could reduce the activation of innate sensing, interferon generation and/or degradation of saRNA in host cells, THP1 cells were transfected with unmodified vs. modified saRNA and the cell supernatant harvested at 24 and 48 h post transfection to characterize the induced cytokine profile before performing a luciferase assay to determine levels of antigen expression. THP1 cells were chosen for this analysis because they represent monocyte cells with very robust innate responses. A Meso Scale Discovery (MSD) assay was used to measure the cytokine levels where the cytokine panel selected for this experiment included ENA-78, GM-CSF, IFN-β, IL-1α, IL-1β, IP-10, MCP-1, MIP-1α, MIP-1β, MDC, Eotaxin-2, GRO-α and MCP-3. As before, luciferase expression was higher with 2′-O-Methyl-GTP modified saRNA compared to both unmodified and N1-Methylpseudo-UTP modified saRNA (see FIG. 46A). Assessment of secreted cytokines and chemokines from THP1 cells transfected with unmodified vs. modified saRNA showed that all modified saRNA had displayed an appreciably muted inflammatory responses compared to unmodified saRNA. Interestingly, this was modest evident for N1-MethylpseudoUTP modified saRNA even though N1-MethylpseudoUTP modification failed to show and enhancement in antigen expression (see FIG. 46B). However, 2′-O-Methyl-GTP modified saRNA also demonstrated a diminished inflammatory response in comparison to unmodified saRNA, although this was less pronounced than with N1-MethylpseudoUTP. This suggests that the enhanced antigen expression of saRNA elicited by 2′-O-Methyl-GTP modification may not be solely due to the reduced innate response, but other mechanisms such as slower degradation.

### Example 23—2′-O-Methyl-Modification Enhances Expression of mRNA Encoding eGFP

Having determined that 2′-O-Methyl-modification provided a major impact on fLuc expression, the inventors determined whether the same effect was observed if the encoded GOI was changed. mRNA encoding eGFP was generated by IVT: using unmodified NTPs; where UTP was replaced with 100% N-1 methylpseudouridine, or where NTPs were substituted by 2′-O-methyl modified NTPs (75% Gm, 75% Am, 90% Cm, 90% Um) and labelled as 2′-O-methyl combo. These constructs were used to transfect HELA and THP1 cells, and at 24 h, cells were harvested, stained and eGFP expression assessed by flow cytometry. 2′-O-Methyl-modified mRNA elicits a higher number of eGFP positive cells and increased eGFP median florescent intensity (MFI) compared to unmodified and N1-Methypseudo-UTP modified mRNA in both HELA (see FIG. 47A) and THP1 (see FIG. 47B) cells. These data indicate that the beneficial effects of 2′-O-Methyl-modification are applicable to different encoded GOIs. Importantly, N1-Methylpseudo-UTP modified mRNA did enhance eGFP express in both cell lines in line with published findings for conventional mRNA, although to a lesser extent than 2′O′methyl combo modification. These data highlight the surprising observation that different modifications are required for beneficial modifications of saRNA relative to mRNA (see also FIG. 41 and Example 19).

### Example 24—2′-O-Methyl-Modified mRNA is Less Inflammatory than Unmodified mRNA

To determine whether the 2′-O-Methyl modification could reduce the activation of innate sensing, interferon generation and/or degradation of mRNA in host cells, THP1 cells, were transfected with unmodified vs. modified mRNA and the cell supernatant harvested at 24 and 48 h post transfection to characterize the induced cytokine profile before performing a luciferase assay to determine levels of antigen expression. THP1 cells were chosen for this analysis because they represent monocyte cells with very robust innate responses. A Meso Scale Discovery (MSD) assay was used to measure the cytokine levels where the cytokine panel selected for this experiment included ENA-78, GM-CSF, IFN-β, IL-1α, IL-1β, IP-10, MCP-1, MIP-1α, MIP-1β, MDC, Eotaxin-2, GRO-α and MCP-3. As before, luciferase expression was higher with 2′-O-Methyl-modified mRNA compared to both unmodified and N1-Methylpseudo-UTP modified mRNA (see FIG. 48A). However, N1-methylpseudouridine modified mRNA was higher that unmodified mRNA in line with previous reports for mRNA Assessment of secreted cytokines and chemokines from THP1 cells transfected with unmodified vs. modified mRNA showed that all modified mRNA had displayed an appreciably muted inflammatory responses compared to unmodified mRNA (see FIG. 48B). Interestingly, this was similar for both N1-MethylpseudoUTP and modified mRNA even though fLuc expression was lower for N1-MethylpseudoUTP modified mRNA than for 2′-O-methyl modified mRNA (see FIG. 48A). These data indicate that the enhanced antigen expression of mRNA elicited by 2′-O-Methyl-modification may not be solely due to the reduced innate response, but other mechanisms such as slower degradation.

### Example 25—2′-O-Methyl-GTP Enhances saRNA Expression In Vivo

Small animal studies were performed to determine the impact of 2′-O-methyl modified saRNA on the duration of expression in vivo. Groups of five female Balb/c mice were injected with 10 μg of LNP formulated VEEV-Fluc saRNA generated with either unmodified NTPs or substituting 75% 2′-O-Methyl-GTP. A PBS group was run as a comparative control. The LNP used for these studies was formulated using C12-200 ionizable lipid with DSPC, cholesterol and DMPE-PEG200. Mice were imaged on day 1 and 5 (day of peak saRNA expression) using an IVIS Spectrum In Vivo Imaging System (FIG. 49A). Expression was observed as early as day 1 with 75% 2′-O-Methyl-GTP modified group, eliciting higher expression than the unmodified group. Stronger levels of expression were also observed by day 5 with similar trends seen on day 1. An Aura Imaging Software was used to analyse and quantify the total expression as photons per second (p/s) (FIG. 49B), in which 2′-O-Methyl-GTP modified VEEV-Fluc showed significantly higher expression compared to unmodified GTP at day 1 where expression gradually decreased over time. These data demonstrate the influence of 2′-O-Methyl-GTP modification alone, further gains in express would be anticipated when using combinations of 2-O-methyl NTPs (Gm+Cm+Am+Um) as predicted by in vitro studies.

### Example 26—2′-O-Methyl-NTPs Enhance mRNA Expression In Vivo

Small animal studies were performed to determine the impact of 2′-O-methyl modified mRNA on the duration of expression in vivo. Groups of five female Balb/c mice were injected with 10 μg of LNP formulated mRNA generated either using unmodified NTPs, where UTP was substituted for N1-Methylpseudouridine or where WT NTPs were substituted by 2′-O-methyl modified NTPs (75% Gm, 75% Am, 90% Cm, 90% Um) and labelled as 2′-O-methyl combo (FIG. 50). The LNP used for these studies was formulated using C12-200 ionizable lipid with DSPC, cholesterol and DMPE-PEG200. Mice were imaged on days 1-3 using an IVIS Spectrum In Vivo Imaging System (FIG. 50A). Expression was observed as early as day 1 with the 2′-O-Methyl-combo modified group, eliciting higher expression than both the unmodified and N1-methylpseudouridine groups. Stronger levels of expression were also observed at days 2 and 3 with similar trends seen on day 1. An Aura Imaging Software was used to analyse and quantify the total expression as photons per second (p/s) (FIG. 50B), in which 2′-O-Methyl-combo modified mRNA showed significantly higher expression compared to unmodified mRNA at days 1-3. These data demonstrate the positive influence of 2′-O-Methyl-modification on mRNA expression.

### Example 27—2′-O-Methyl-GTP Enhances the Immunogenicity of saRNA In Vivo

Small animal studies were performed to determine the impact of 2′-O-methyl modified saRNA on immunogenicity in vivo. VEEV saRNA encoding the hemagglutinin protein of influenza (VEEV-Ha) downstream of the sub-genomic promotor was generated by IVT: using unmodified NTPs; where GTP was substituted with ≥75% 2′-O-methyl modified NTP; or where UTP was replaced with 100% N-1 methylpseudouridine. Four groups of five female Balb/c mice were immunised intramuscularly with 10 μg of LNP formulated VEEV-Ha saRNA at week o and 4 with 1) unmodified saRNA, 2) 100% N1-MethylpseudoUTP modified saRNA, 3) 75% 2′-O-Methyl-GTP modified saRNA and 4) PBS (FIG. 51A). At week 6, mice were bled and euthanised. An HA ELISA was performed to assess total HA specific IgG. The 2′-O-Methyl-GTP modified group had significantly higher antibody responses after the boost compared to both unmodified and N1-MethylpseudoUTP modified groups (FIG. 51A). Microneutralization assay was also performed on the mice sera using Influenza A California 2009 H1N1 virus strain (FIG. 51B) and results were in accordance with the ELISA data, whereby the neutralizing antibody responses were significantly higher in the 2′-O-Methyl-GTP modified group after boost compared to all other groups. These data demonstrate the influence of 2′-O-Methyl-GTP modification alone on immunogenicity, additional influence would be anticipated when using combinations of 2-O-methyl NTPs (Gm+Cm+Am+Um) as predicted by in vitro studies.

### REFERENCES

1. Morais P, Adachi H, Yu Y T. The Critical Contribution of Pseudouridine to mRNA COVID-19 Vaccines. Front Cell Dev Biol. 2021 Nov. 4; 9:789427. doi: 10.3389/fcell.2021.789427. PMID: 34805188; PMCID: PMC8600071.
2. Kariko K, Buckstein M, Ni H, Weissman D. Suppression of RNA recognition by Toll-like receptors: the impact of nucleoside modification and the evolutionary origin of RNA. Immunity. 2005 August; 23 (2): 165-75. doi: 10.1016/j.immuni.2005.06.008. PMID: 16111635.
3. Andries O, Mc Cafferty S, De Smedt S C, Weiss R, Sanders N N, Kitada T. N (1)-methylpseudouridine-incorporated mRNA outperforms pseudouridine-incorporated mRNA by providing enhanced protein expression and reduced immunogenicity in mammalian cell lines and mice. J Control Release. 2015 Nov. 10; 217:337-44. doi: 10.1016/j.jconrel.2015.08.051. Epub 2015 Sep. 3. PMID: 26342664.
4. Mccown P J, Ruszkowska A, Kunkler C N, Breger K, Hulewicz J P, Wang M C, Springer N A, Brown J A. Naturally occurring modified ribonucleosides. Wiley Interdiscip Rev RNA. 2020 September; 11 (5): e1595. doi: 10.1002/wrna.1595. Epub 2020 Apr. 16. PMID: 32301288; PMCID: PMC7694415.
5. Burmeister P E, Lewis S D, Silva R F, Preiss J R, Horwitz L R, Pendergrast P S, McCauley T G, Kurz J C, Epstein D M, Wilson C, Keefe A D. Direct in vitro selection of a 2′-O-methyl aptamer to VEGF. Chem Biol. 2005 January; 12 (1): 25-33. doi: 10.1016/j.chembiol.2004.10.017. PMID: 15664512.
6. Freund I, Eigenbrod T, Helm M, Dalpke A H. RNA Modifications Modulate Activation of Innate Toll-Like Receptors. Genes (Basel). 2019 Jan. 29; 10 (2): 92. doi: 10.3390/genes10020092. PMID: 30699960; PMCID: PMC6410116.
7. Züst R, Cervantes-Barragan L, Habjan M, Maier R, Neuman B W, Ziebuhr J, Szretter K J, Baker S C, Barchet W, Diamond M S, Siddell S G, Ludewig B, Thiel V. Ribose 2′-O-methylation provides a molecular signature for the distinction of self and non-self mRNA dependent on the RNA sensor Mda5. Nat Immunol. 2011 February; 12 (2): 137-43. doi: 10.1038/ni.1979. Epub 2011 Jan. 9. PMID: 21217758; PMCID: PMC3182538.
8. Robbins M, Judge A, Liang L, Mcclintock K, Yaworski E, MacLachlan I. 2′-O-methyl-modified RNAs act as TLR7 antagonists. Mol Ther. 2007 September; 15 (9): 1663-9. doi: 10.1038/sj.mt.6300240. Epub 2007 Jun. 19. PMID: 17579574.
9. Lee J, Chuang T H, Redecke V, She L, Pitha P M, Carson D A, Raz E, Cottam H B. Molecular basis for the immunostimulatory activity of guanine nucleoside analogs: activation of Toll-like receptor 7. Proc Natl Acad Sci USA. 2003 May 27; 100 (11): 6646-51. doi: 10.1073/pnas.0631696100. Epub 2003 May 8. PMID: 12738885; PMCID: PMC164501.
10. Lee Y, Urban J H, Xu L, Sullenger B A, Lee J. 2′Fluoro Modification Differentially Modulates the Ability of RNAs to Activate Pattern Recognition Receptors. Nucleic Acid Ther. 2016 June; 26 (3): 173-82. doi: 10.1089/nat.2015.0575. Epub 2016 Jan. 20. PMID: 26789413; PMCID: PMC4900224.
11. Mu X, Greenwald E, Ahmad S, Hur S. An origin of the immunogenicity of in vitro transcribed RNA. Nucleic Acids Res. 2018 Jun. 1; 46 (10): 5239-5249.
12. Damase T R, Sukhovershin R, Boada C, Taraballi F, Pettigrew R I, Cooke J P. The Limitless Future of RNA Therapeutics. Front Bioeng Biotechnol. 2021 Mar. 18; 9:628137.
13. Ayadi L, et al. RNA ribose methylation (2′-O-methylation): Occurrence, biosynthesis and biological functions. Biochim Biophys Acta Gene Regul Mech. 2019; 1862:253-269.
14. Hoernes T P, et al. Nucleotide modifications within bacterial messenger RNAs regulate their translation and are able to rewire the genetic code. Nucleic Acids Res. 2016; 44:852-62.
15. Hoernes T P, et al. Eukaryotic Translation Elongation is Modulated by Single Natural Nucleotide Derivatives in the Coding Sequences of mRNAs. Genes (Basel). 2019; 10:84.
16. J. Choi, et al. 2′-O-methylation in mRNA disrupts tRNA decoding during translation elongation, Nat. Struct. Mol. Biol. 2018; 25:208-216.
17. Elliott B A, et al. Modification of messenger RNA by 2′-O-methylation regulates gene expression in vivo. Nat Commun. 2019; 10:3401.
18. Höfler S, Carlomagno T. Structural and functional roles of 2′-O-ribose methylations and their enzymatic machinery across multiple classes of RNAs. Curr Opin Struct Biol. 2020; 65:42-50.
19. Karikó K, et al. Incorporation of pseudouridine into mRNA yields superior nonimmunogenic vector with increased translational capacity and biological stability. Mol Ther. 2008; 16:1833-40.
20. Kratschmer C, Levy M. Effect of Chemical Modifications on Aptamer Stability in Serum. Nucleic Acid Ther. 2017; 27:335-344.
21. Burmeister P E, et al. Direct in vitro selection of a 2′-O-methyl aptamer to VEGF. Chem Biol. 2005; 12:25-33.
22. Abou Assi H, et al. 2′-O-Methylation can increase the abundance and lifetime of alternative RNA conformational states. Nucleic Acids Res. 2020; 48:12365-12379.
23. Gebhardt A, et al. Discrimination of Self and Non-Self Ribonucleic Acids. J Interferon Cytokine Res. 2017; 37:184-197.
24. Sioud M, et al. Suppression of immunostimulatory siRNA-driven innate immune activation by 2′-modified RNAs. Biochem Biophys Res Commun. 2007; 361 (1): 122-6.
25. Gao M, et al. Synthetic modified messenger RNA for therapeutic applications. Acta Biomater. 2021; 131:1-15.
26. Robbins M, et al., 2′-O-methyl-modified RNAs act as TLR7 antagonists. Mol Ther. 2007; 151663-9.
27. Lee Y, et al. 2′Fluoro Modification Differentially Modulates the Ability of RNAs to Activate Pattern Recognition Receptors. Nucleic Acid Ther. 2016; 26:173-82.
28. T. P. Hoernes, N. Clementi, K. Faserl, H. Glasner, K. Breuker, H. Lindner, A. Hüttenhofer, M. D. Erlacher, Nucleotide modifications within bacterial messenger RNAs regulate their translation and are able to rewire the genetic code, Nucleic Acids Res. 44 (2) (2016) 852-862.
29. Hertler J, Slama K, Schober B, Özrendeci Z, Marchand V, Motorin Y, Helm M. Synthesis of point-modified mRNA. Nucleic Acids Res. 2022 Nov. 11; 50 (20): e115. doi: 10.1093/nar/gkac719.
30. (Kariko, K. at al., (2005). Suppression of RNA recognition by toll-like receptors: The impact of nucleoside modification and the evolutionary origin of RNA. Immunity, 23, 165-175.
31. Pardi N. et al., Expression kinetics of nucleoside-modified mRNA delivered in lipid nanoparticles to mice by various routes. J Control Release. 2015 Nov. 10; 217:345-51).
32. Takahashi S, Okura H, Sugimoto N. Bisubstrate Function of RNA Polymerases Triggered by Molecular Crowding Conditions. Biochemistry 2019, 58, 1081-1093.

The following clauses describe embodiments of the invention:
1. A method of preparing a modified RNA molecule, wherein the method comprises contacting, in the presence of at least 20 mM magnesium ions, (i) a template nucleic acid sequence, (ii) an RNA polymerase, and (iii) a plurality of nucleotide triphosphates (NTPs), one or more of which is a modified nucleotide triphosphate (NTP), wherein the RNA polymerase transcribes the template nucleic acid sequence to form an RNA molecule comprising at least 20 nucleotides, and wherein at least 25% of the constituent nucleotides in the RNA molecule are modified.
2. Use of 20 mM magnesium ions in a transcription reaction to prepare a modified RNA molecule comprising at least 20 nucleotides, wherein at least 25% of the constituent nucleotides in the RNA molecule are modified.
3. A method or use according to any preceding clause, wherein the method comprises the use of:
(i) at least 30 mM magnesium ions, at least 40 mM magnesium ions, at least 50 mM magnesium ions, at least 60 mM magnesium ions, or at least 70 mM magnesium ions; or
(ii) between 50 mM and 100 mM magnesium ions, between 60 mM and 95 mM magnesium ions, between 65 mM and 90 mM magnesium ions, between 70 mM and 80 mM magnesium ions, or between 71 mM and 79 mM magnesium ions.
4. A method or use according to any preceding clause, wherein the magnesium ions are provided as Mg²⁺ ions.
5. A method or use according to any preceding clause, wherein the magnesium ions are provided as magnesium acetate.
6. A method or use according to any preceding clause, wherein the RNA is selected from a group consisting of: messenger RNA (mRNA), micro RNA (miRNA); short interfering RNA (siRNA); short hairpin RNA (shRNA); anti-sense RNA; RNA aptamers; self-amplifying RNA (saRNA); interference RNA (RNAi); non-coding RNA; circular RNA; and small RNA.
7. A method or use according to any preceding clause, wherein the RNA is self-amplifying RNA (saRNA) or messenger RNA (mRNA).
8. A method or use according to any preceding clause, wherein the RNA molecule is:
   (i) at least 20, 21, 22 or 23 bases in length;
   (ii) at least 24, 25, 26 or 27 bases in length;
   (iii) at least 28, 29, 30, or 31 bases in length; or
   (iv) at least 32, 33, 34 or 35 bases in length.
9. A method or use according to any preceding clause, wherein the RNA molecule is:
   (i) at least at least 50 bases in length, at least 60 bases in length, at least 75 bases in length, at least 100 bases in length, at least 200 bases in length, at least 300 bases in length, at least 400 bases in length, at least 500 bases in length, at least 600 bases in length, at least 700 bases in length, at least 800 bases in length, or at least 900 bases in length;
   (ii) at least 1000 bases in length, at least 2000 bases in length, at least 3000 bases in length, at least 4000 bases in length, at least 5000 bases in length, at least 6000 bases in length, at least 7000 bases in length, at least 8000 bases in length, at least 9000 bases in length, at least 10,000 bases in length, at least 11,000 bases in length or at least 12000 bases in length.
10. A method or use according to any preceding clause, wherein the method comprises the use of an RNA polymerase, which is selected from a group consisting of: T7; T3; SP6; KP34; Syn5; or other DNA-dependent RNA polymerase; or a mutated variant of any of these RNA polymerases.
11. A method or use according to any preceding clause, wherein the method comprises the use of the plurality of nucleotide triphosphates at a concentration of:
   (i) at least 1 mM, 2 mM, 3 mM or 4 mM;
   (ii) at least 5 mM, 6 mM or 7 mM; or
   (iii) at least 8 mM, 9 mM, 10 mM.
12. A method or use according to any preceding clause, wherein the one or more modified NTP is selected from a group consisting of: a modified adenosine-5′-triphosphate (ATP); a modified cytidine-5′-triphosphate (CTP); a modified guanosine-5′-triphosphate (GTP) and a modified uridine-5′-triphosphate (UTP).
13. A method or use according to any preceding clause, wherein the method comprises using at least one, two, three, four or five modified NTP selected from a group consisting of: a modified ATP, a modified CTP, a modified GTP, and/or a modified UTP.
14. A method or use according to any preceding clause, wherein the one or more modified NTP comprises a 2′-substituted group in which the OH group normally at the 2′ position is replaced with a halogen, an optionally substituted aromatic group, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.
15. A method or use according to any preceding clause, wherein the one or more modified NTP comprises a substituted nucleobase, wherein the nucleobase is substituted with a halogen, an optionally substituted aromatic group, a N₃, an OH, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl, optionally wherein, alternatively, or additionally, an oxo group in the nucleobase is replaced by a ═S group.
16. A method or use according to any one of clauses 14-15, wherein the alkyl, alkenyl or alkynyl is an optionally substituted C₁-C₂₀ alkyl, alkenyl or alkynyl NTP, wherein the alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl, preferably wherein the alkyl, alkenyl or alkynyl is an optionally substituted C₁-C₁₀ alkyl, alkenyl or alkynyl NTP, wherein the alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.
17. A method or use according to any one of clauses 14-16, wherein the alkyl, alkenyl or alkynyl is an optionally substituted C₁₋₆ alkyl, a C₂₋₆ alkenyl or a C₂₋₆ alkynyl, wherein the, or each alkyl, alkenyl or alkynyl is optionally substituted with halogen, OXO, OR, CN, NR₂ or SR, wherein R is H or C₁₋₆ alkyl, C₂₋₆ alkenyl or C₂₋₆ alkynyl, preferably wherein the alkyl, alkenyl or alkynyl is an optionally substituted C₁₋₃ alkyl, a C₂₋₃ alkenyl or a C₂₋₃ alkynyl, wherein the, or each alkyl, alkenyl or alkynyl is substituted with one or more substituents selected from the group consisting of an oxo, OH, OMe, NH₂ and NRH, wherein R is C₁₋₆ alkyl, C₂₋₆ alkenyl or C₂₋₆ alkynyl.
18. A method or use according to any preceding clause, wherein the one or more modified NTP comprises a 2′-methyl modified NTP.
19. A method or use according to any preceding clause, wherein the one or more modified NTP comprises a 2′-O-methyl modified NTP.
20. A method or use according to any preceding clause, wherein the one or more modified NTP is selected from a group consisting of: 2′-O-methyladenosine (Am); 1,2′-dimethyl-adenosine (m¹Am); N6,2′-O-dimethyl-adenosine (m⁶Am); N⁶,N⁶,2-O-trimethyl-adenosine (m^{6,6}Am); 2′-O-methylinosine (Im); 1,2′-O-dimethylinosine (m¹Im); 2′-O-methylguanosine (Gm); 1,2′-O-dimethylguanosine (m¹Gm); N2,2′-O-dimethylguanosine (m²Gm); N2,N2, 2′-O-trimethylguanosine (m^{2,2}Gm); N2,7,2′-O-trimethyl-guanosine (m^{2,7}Gm); 2′-O-methylcytidine; N⁴,2′-O-dimethyl cytidine (m⁴Cm); N⁴,N⁴,2-O-trimethyl-cytidine (m^{4,4} Cm); 5,2′-O-dimethyl-cytidine (m⁵Cm); N⁴-acetyl-2′-O-methyl-cytidine (ac⁴Cm); 2′-O-methyl-5-hydromethyl-cytidine (hm⁵Cm); 5-formyl-2′-O-methyl-cytidine (f⁵Cm); 2′-O-methyluridine (Um); 3,2′-O-dimethyluridine (m³Um); 5,2′-O-dimethyluridine (m⁵Um or Tm); 2-thio-2′-O-methyl-uridine (s²Um); 2′-O-methyl-pseudouridine (Ym); 5-carbamoylmethyl-2′-O-methyluridine (ncm⁵Um); 5-methoxycarbonylmethyl-2′-O-methyluridine (mcm⁵Um); 5-(carboxyhydroxymethyl)-2′-O-methyluridine methyl ester (mchm⁵Um); 5-carboxymethylaminomethyl-2′-O-methyluridine (cmbm⁵Um); 2′-O-methyluridine 5-oxyacetic acid methyl ester (mcmo⁵Um); 5-(isopentenylaminomethyl)-3′O-methyluridine (inm⁵Um); and any other synthetic NTP that contains a 2′-O-methylated ribose.
21. A method or use according to any preceding clause, wherein the one or more modified NTP comprises a 2′-O-methyl modified ATP, a 2′-O-methyl modified CTP, a 2′-O-methyl modified GTP, and/or a 2′-O-methyl modified UTP.
22. A method or use according to any preceding clause, wherein the method comprises using at least one, two, three or four modified NTP selected from a group consisting of: 2′-O-methyl-ATP, a 2′-O-methyl-CTP, a 2′-O-methyl-GTP, and a 2′-O-methyl-UTP.
23. A method or use according to any preceding clause, wherein when the modified RNA molecule is saRNA, the RNA molecule comprises modified GTP, preferably modified 2′-O-methyl-GTP.
24. A method or use according to any preceding clause, wherein when the modified RNA molecule is mRNA, the RNA molecule comprises modified CTP and modified GTP, preferably modified 2′-O-methyl CTP and modified 2′-O-methyl-GTP.
25. A method or use according to any preceding clause, wherein at least 30%, 35% or 40% of the constituent nucleotides in the resultant RNA molecule are modified, wherein the modified nucleotides comprise adenine, cytosine, guanine and/or uracil; preferably wherein at least 45%, 50% or 55% of the constituent nucleotides in the resultant RNA molecule are modified, wherein the modified nucleotides comprise adenine, cytosine, guanine and/or uracil.
26. A method or use according to any preceding clause, wherein the method comprises combining one or more 2-O-methyl modified NTP with one or more other modified NTP bases, which is not a 2-O-methyl modified NTP.
26. A method or use according to any preceding clause, wherein the method comprises combining:
   (i) one or more modified NTP comprising a 2′-substituted group in which the OH group normally at the 2′ position is replaced with a halogen, an optionally substituted aromatic group, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl; and
   (ii) one or more modified NTP, which is not a 2′-O-methyl modified NTP.
27. A method or use according to any preceding clause, wherein the template nucleic acid sequence comprises DNA, optionally the template nucleic acid comprises a plasmid.
28. A method or use according to any preceding clause, wherein the template nucleic acid is transcribed to create the resultant modified RNA molecule, which is selected from a group consisting of: messenger RNA (mRNA), micro RNA (miRNA); short interfering RNA (siRNA); short hairpin RNA (shRNA); anti-sense RNA; RNA aptamer; self-amplifying RNA (saRNA); interference RNA (RNAi); non-coding RNA; circular RNA; and small RNA.
29. A method or use according to any preceding clause, wherein the template nucleic acid sequence encodes an antigen which is derived from a virus, a bacteria, a mycoplasma, a fungus, an animal, a plant, an alga, a parasite, or a protozoan, or other organism which causes a disease in a subject, preferably a human or animal.
30. A method or use according to any preceding clause, wherein the template nucleic acid encodes an innate inhibitor protein, which counteracts the innate immune response in a subject administered with a vaccine comprising the resultant RNA molecule.
31. An RNA molecule obtained or obtainable by the method according to any one of clauses 1-30.
32. A pharmaceutical composition comprising the RNA molecule according to clause 31 and a pharmaceutically acceptable vehicle.
33. A method of preparing the pharmaceutical composition according to clause 32, the method comprising contacting the RNA molecule according to clause 31 and a pharmaceutically acceptable vehicle.
34. The RNA molecule according to clause 31, or the pharmaceutical composition according to clause 32, for use as a medicament.
35. The RNA molecule according to clause 31, or the pharmaceutical composition according to clause 32, for use in treating, preventing or ameliorating a disease in a subject.
36. A vaccine composition comprising the RNA molecule according to clause 31, or the pharmaceutical composition according to clause 32.
37. The RNA molecule according to clause 31, the pharmaceutical composition according to clause 32 or the vaccine according to clause 36, for use in stimulating an immune response in a subject.
38. One or more modified nucleotide triphosphate (NTP), for use in:
   (i) enhancing the expression and/or translation of an RNA molecule comprising the one or more modified NTP;
   (ii) enhancing the stability of an RNA molecule comprising the one or more modified NTP; and/or
   (iii) reducing the activation of innate sensing, interferon generation and/or degradation of an RNA molecule comprising the one or more modified NTP,
wherein the one or more modified NTP comprises a 2′-substituted group in which the OH group normally at the 2′ position is replaced with a halogen, an optionally substituted aromatic group, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.
39. One or more modified NTP, for use according to clause 38, wherein the use comprises contacting, in the presence of at least 20 mM magnesium ions, (i) a template nucleic acid sequence, (ii) an RNA polymerase, and (iii) a plurality of nucleotide triphosphates (NTPs), one or more of which is a modified nucleotide triphosphate (NTP), wherein the RNA polymerase transcribes the template nucleic acid sequence to form the RNA molecule.
40. One or more modified NTP, for use according to either clause 38 or 39, wherein the one or more modified NTP is incorporated into the RNA molecule using the method according to any one of clauses 1-30.
41. One or more modified NTP, for use according to any one of clauses 38-40, wherein the one or more modified NTP is as defined in any one of clauses 1-30.
42. One or more modified NTP, for use according to any one of clauses 38-41, wherein the one or more modified NTP comprises a 2′-O-methyl modified ATP, a 2′-O-methyl modified CTP, a 2′-O-methyl modified GTP, and/or a 2′-O-methyl modified UTP.
43. One or more modified NTP, for use according to any one of clauses 38-42, wherein the RNA is selected from a group consisting of: messenger RNA (mRNA); self-amplifying RNA (saRNA); micro RNA (miRNA); short interfering RNA (siRNA); short hairpin RNA (shRNA); anti-sense RNA; RNA aptamers; interference RNA (RNAi); non-coding RNA; circular RNA; and small RNA.
44. One or more modified NTP, for use according to any one of clauses 38-43, wherein the RNA molecule is saRNA, optionally wherein the RNA molecule comprises modified 2′-O-methyl-GTP.
45. One or more modified NTP, for use according to any one of clauses 38-44, wherein the RNA molecule is mRNA, optionally wherein the RNA molecule comprises modified 2′-O-methyl-GTP and modified 2′-O-methyl CTP.
46. One or more modified NTP, for use according to any one of clauses 38-45, wherein the length of the RNA molecule is as defined in either clause 8 or clause 9.
47. One or more 2′-O-methyl modified nucleotide triphosphate (NTP) in an RNA molecule, for use in enhancing the translation of the RNA molecule.

1. One or more modified nucleotide triphosphate (NTP), for use in:
(i) enhancing the expression and/or translation of an RNA molecule comprising the one or more modified NTP;
(ii) enhancing the stability of an RNA molecule comprising the one or more modified NTP; and/or
(iii) reducing the activation of innate sensing, interferon generation and/or degradation of an RNA molecule comprising the one or more modified NTP,
wherein the one or more modified NTP comprises a 2′-substituted group in which the OH group normally at the 2′ position is replaced with a halogen, an optionally substituted aromatic group, a NH₂, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.

2. One or more modified NTP, for use according to claim 1, wherein the RNA molecule is selected from a group consisting of: messenger RNA (mRNA), micro RNA (miRNA); short interfering RNA (siRNA); short hairpin RNA (shRNA); anti-sense RNA; RNA aptamers; self-amplifying RNA (saRNA); interference RNA (RNAi); non-coding RNA; circular RNA; and small RNA.

3. One or more modified NTP, for use according to either claim 1 or claim 2, wherein the RNA molecule is self-amplifying RNA (saRNA).

4. One or more modified NTP, for use according to either claim 1 or claim 2, wherein the RNA molecule is messenger RNA (mRNA).

5. One or more modified NTP, for use according to any preceding claim, wherein the one or more modified NTP is selected from a group consisting of: a modified adenosine-5′-triphosphate (ATP); a modified cytidine-5′-triphosphate (CTP); a modified guanosine-5′-triphosphate (GTP), a modified uridine-5′-triphosphate (UTP), and a modified thymidine-5′-triphosphate (TTP).

6. One or more modified NTP, for use according to any preceding claim, wherein the one or more modified NTP comprises at least two, three, four or five modified NTP selected from a group consisting of: a modified ATP, a modified CTP, a modified GTP, a modified UTP and/or a modified TTP.

7. One or more modified NTP, for use according to any preceding claim, wherein the one or more modified NTP comprises a substituted nucleobase, wherein the nucleobase is substituted with a halogen, an optionally substituted aromatic group, a NH₂, a N₃, an OH, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl, optionally wherein, alternatively, or additionally, an oxo group in the nucleobase is replaced by a ═S group.

8. One or more modified NTP, for use according to any preceding claim, wherein the alkyl, alkenyl or alkynyl is an optionally substituted C₁-C₂₀ alkyl, alkenyl or alkynyl NTP, wherein the alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl, preferably wherein the alkyl, alkenyl or alkynyl is an optionally substituted C₁-C₁₀ alkyl, alkenyl or alkynyl NTP, wherein the alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl.

9. One or more modified NTP, for use according to any preceding claim, wherein the alkyl, alkenyl or alkynyl is an optionally substituted C₁₋₆ alkyl, a C₂₋₆ alkenyl or a C₂₋₆ alkynyl, wherein the, or each alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl, preferably wherein the alkyl, alkenyl or alkynyl is an optionally substituted C₁₋₃ alkyl, a C₂₋₃ alkenyl or a C₂₋₃ alkynyl, wherein the, or each alkyl, alkenyl or alkynyl is substituted with one or more substituents selected from the group consisting of an oxo, OH, OMe, NH₂ and NRH, wherein R is C₁₋₆ alkyl, C₂₋₆ alkenyl or C₂₋₆ alkynyl.

10. One or more modified NTP, for use according to any preceding claim, wherein the one or more modified NTP comprises a 2′-methyl modified NTP.

11. One or more modified NTP, for use according to any preceding claim, wherein the one or more modified NTP comprises a 2′-O-methyl modified NTP.

12. One or more modified NTP, for use according to any preceding claim, wherein the one or more modified NTP is selected from a group consisting of: 2′-O-methyladenosine (Am); 1,2′-dimethyl-adenosine (m¹Am); N6,2′-O-dimethyl-adenosine (m⁶Am); N⁶,N⁶,2-O-trimethyl-adenosine (m^{6,6}Am); 2′-O-methylinosine (Im); 1,2′-O-dimethylinosine (m¹Im); 2′-O-methylguanosine (Gm); 1,2′-O-dimethylguanosine (m¹Gm); N2,2′-O-dimethylguanosine (m²Gm); N2,N2, 2′-O-trimethylguanosine (m^{2,2}Gm); N2,7,2′-O-trimethyl-guanosine (m^{2,7}Gm); 2′-O-methylcytidine; N⁴,2′-O-dimethyl cytidine (m⁴Cm); N⁴,N⁴,2-O-trimethyl-cytidine (m^{4,4} Cm); 5,2′-O-dimethyl-cytidine (m⁵Cm); N⁴-acetyl-2′-O-methyl-cytidine (ac⁴Cm); 2′-O-methyl-5-hydromethyl-cytidine (hm⁵Cm); 5-formyl-2′-O-methyl-cytidine (f⁵Cm); 2′-O-methyluridine (Um); 3,2′-O-dimethyluridine (m³Um); 5,2′-O-dimethyluridine (m⁵Um or Tm); 2-thio-2′-O-methyl-uridine (s²Um); 2′-O-methyl-pseudouridine (Ym); 5-carbamoylmethyl-2′-O-methyluridine (ncm⁵Um); 5-methoxycarbonylmethyl-2′-O-methyluridine (mcm⁵Um); 5-(carboxyhydroxymethyl)-2′-O-methyluridine methyl ester (mchm⁵Um); 5-carboxymethylaminomethyl-2′-O-methyluridine (cmbm⁵Um); 2′-O-methyluridine 5-oxyacetic acid methyl ester (mcmo⁵Um); 5-(isopentenylaminomethyl)-3′O-methyluridine (inm⁵Um); and any other synthetic NTP that contains a 2′-O-methylated ribose.

13. One or more modified NTP, for use according to any preceding claim, wherein the one modified NTP is selected from a group consisting of: 2′-O-methyl-ATP, a 2′-O-methyl-CTP, a 2′-O-methyl-GTP, and a 2′-O-methyl-UTP.

14. One or more modified NTP, for use according to any preceding claim, wherein at least two modified NTPs are selected from a group consisting of: 2′-O-methyl-ATP, a 2′-O-methyl-CTP, a 2′-O-methyl-GTP, and a 2′-O-methyl-UTP.

15. One or more modified NTP, for use according to any preceding claim, wherein at least three modified NTPs are selected from a group consisting of: 2′-O-methyl-ATP, a 2′-O-methyl-CTP, a 2′-O-methyl-GTP, and a 2′-O-methyl-UTP.

16. One or more modified NTP, for use according to any preceding claim, wherein at least four modified NTPs are selected from a group consisting of: 2′-O-methyl-ATP, a 2′-O-methyl-CTP, a 2′-O-methyl-GTP, and a 2′-O-methyl-UTP.

17. One or more modified NTP, for use according to any preceding claim, wherein when the RNA molecule is saRNA, the RNA molecule comprises modified GTP, preferably modified 2′-O-methyl-GTP.

18. One or more modified NTP, for use according to any preceding claim, wherein when the RNA molecule is mRNA, the RNA molecule comprises modified CTP and modified GTP, preferably modified 2′-O-methyl CTP and modified 2′-O-methyl-GTP.

19. One or more modified NTP, for use according to any preceding claim, wherein at least 30%, 35% or 40% of the constituent nucleotides in the RNA molecule are modified, wherein the modified nucleotides comprise adenine, cytosine, guanine and/or uracil; preferably wherein at least 45%, 50% or 55% of the constituent nucleotides in the RNA molecule are modified, wherein the modified nucleotides comprise adenine, cytosine, guanine and/or uracil.

20. One or more modified NTP, for use according to any preceding claim, wherein one or more 2-O-methyl modified NTP is combined with one or more other modified NTP bases, which is not a 2-O-methyl modified NTP.

21. One or more modified NTP, for use according to any preceding claim, wherein:
(i) the one or more modified NTP comprising a 2′-substituted group in which the OH group normally at the 2′ position is replaced with a halogen, an optionally substituted aromatic group, a NH₂, a N₃, a H, an optionally substituted O-alkyl, O-alkenyl or O-alkynyl group, or an optionally substituted alkyl, alkenyl or alkynyl group, wherein in each instance the aromatic group, alkyl, alkenyl or alkynyl is optionally substituted with halogen, oxo, OR, CN, NR₂ or SR, wherein R is H or C₁₋₆ alkyl, C₂₋₆ alkenyl or C₂₋₆ alkynyl; is combined with
(ii) one or more modified NTP, which is not a 2′-O-methyl modified NTP.

22. One or more modified NTP, for use according to claim 21, wherein:
(i) the one or more 2-O-methyl modified NTP is selected from a group consisting of a 2′-O-methyl modified ATP (2′-O-methyl-ATP): a 2′-O-methyl modified CTP (2′-O-methyl-CTP); a 2′-O-methyl modified GTP (2′-O-methyl-GTP); and a 2′-O-methyl modified UTP (2′-O-methyl-UTP); is combined with
(ii) one or more other modified NTP bases, which is not a 2-O-methyl modified NTP.

23. One or more modified NTP, for use according to either claim 21 or claim 22, wherein the one or more other modified NTP bases, which is not a 2-O-methyl modified NTP, is:
(i) N′ methyl-pseudouridine, fluorinated UTP, 2′-chloro NTP, 2′-ethyl NTP, 2′-bromo NTP, 2′-amino NTP, 2′-fluoro NTP, or 2′-deoxy NTP; or
(ii) an alternative 2′-modified nucleotide listed in Table 7.

24. One or more modified NTP, for use according to any preceding claim, wherein the RNA molecule is encoded by a template nucleic acid sequence, which comprises DNA.

25. One or more modified NTP, for use according to claim 24, wherein the template nucleic acid sequence encodes an antigen which is derived from a tumour, virus, a bacteria, a mycoplasma, a fungus, an animal, a plant, an alga, a parasite, or a protozoan, or other organism which causes a disease in a subject, preferably a human or animal.

26. One or more modified NTP, for use according to claim 24, wherein the template nucleic acid encodes a therapeutic protein, which is derived from an animal or a human, and which treats, prevents or ameliorates disease in a subject, preferably a human or animal subject.

27. One or more modified NTP, for use according to any one of claims 22-26, wherein the template nucleic acid encodes an innate inhibitor protein, which counteracts the innate immune response in a subject administered with a vaccine comprising the RNA molecule.

28. One or more modified NTP, for use according to any preceding claim, wherein the use comprises contacting, in the presence of at least 20 mM magnesium ions, (i) a template nucleic acid sequence, (ii) an RNA polymerase, and (iii) a plurality of nucleotide triphosphates (NTPs), one or more of which is a modified nucleotide triphosphate (NTP), wherein the RNA polymerase transcribes the template nucleic acid sequence to form the RNA molecule.

29. A method of preparing a modified RNA molecule, wherein the method comprises contacting, in the presence of at least 20 mM magnesium ions, (i) a template nucleic acid sequence, (ii) an RNA polymerase, and (iii) a plurality of nucleotide triphosphates (NTPs), one or more of which is a modified nucleotide triphosphate (NTP), wherein the RNA polymerase transcribes the template nucleic acid sequence to form an RNA molecule comprising at least 20 nucleotides, and wherein at least 25% of the constituent nucleotides in the RNA molecule are modified.

30. Use of 20 mM magnesium ions in a transcription reaction to prepare a modified RNA molecule comprising at least 20 nucleotides, wherein at least 25% of the constituent nucleotides in the RNA molecule are modified.

31. A method or use according to either claim 29 or 30, wherein the method comprises the use of:
(i) at least 30 mM magnesium ions, at least 40 mM magnesium ions, at least 50 mM magnesium ions, at least 60 mM magnesium ions, or at least 70 mM magnesium ions; or
(ii) between 50 mM and 100 mM magnesium ions, between 60 mM and 95 mM magnesium ions, between 65 mM and 90 mM magnesium ions, between 70 mM and 80 mM magnesium ions, or between 71 mM and 79 mM magnesium ions.

32. A method or use according to any one of claims 29-31, wherein the magnesium ions are provided as Mg²⁺ ions, preferably wherein the magnesium ions are provided as magnesium acetate.

33. A method or use according to any one of claims 29-32, wherein the RNA is as defined in any one of claims 2-4.

34. A method or use according to any one of claims 29-33, wherein the RNA molecule is:
(i) at least 20, 21, 22 or 23 bases in length;
(ii) at least 24, 25, 26 or 27 bases in length;
(iii) at least 28, 29, 30, or 31 bases in length; or
(iv) at least 32, 33, 34 or 35 bases in length.

35. A method or use according to any one of claims 29-34, wherein the RNA molecule is:
(i) at least at least 50 bases in length, at least 60 bases in length, at least 75 bases in length, at least 100 bases in length, at least 200 bases in length, at least 300 bases in length, at least 400 bases in length, at least 500 bases in length, at least 600 bases in length, at least 700 bases in length, at least 800 bases in length, or at least 900 bases in length;
(ii) at least 1000 bases in length, at least 2000 bases in length, at least 3000 bases in length, at least 4000 bases in length, at least 5000 bases in length, at least 6000 bases in length, at least 7000 bases in length, at least 8000 bases in length, at least 9000 bases in length, at least 10,000 bases in length, at least 11,000 bases in length or at least 12000 bases in length.

36. A method or use according to any one of claims 29-35, wherein the method comprises the use of an RNA polymerase, which is selected from a group consisting of: T7; T3; SP6; KP34; Syn5; or other DNA-dependent RNA polymerase; or a mutated variant of any of these RNA polymerases.

37. A method or use according to any one of claims 29-36, wherein the method comprises the use of the plurality of nucleotide triphosphates at a concentration of:
(i) at least 1 mM, 2 mM, 3 mM or 4 mM;
(ii) at least 5 mM, 6 mM or 7 mM; or
(iii) at least 8 mM, 9 mM, 10 mM.

38. A method or use according to any one of claims 29-37, wherein the one or more modified NTP is as defined in any one of claims 1-28.

39. A method or use according to any one of claims 29-38, wherein the template nucleic acid sequence is as defined as in any one of claims 24-27.

40. An RNA molecule obtained or obtainable by the method according to any one of claims 29-39.

41. A pharmaceutical composition comprising the RNA molecule according to claim 40, and a pharmaceutically acceptable vehicle.

42. A method of preparing the pharmaceutical composition according to claim 41, the method comprising contacting the RNA molecule according to claim 40 and a pharmaceutically acceptable vehicle.

43. The RNA molecule according to claim 40, or the pharmaceutical composition according to claim 41, for use as a medicament.

44. The RNA molecule according to claim 40, or the pharmaceutical composition according to claim 41, for use in treating, preventing or ameliorating a disease in a subject.

45. A vaccine composition comprising the RNA molecule according to claim 40, or the pharmaceutical composition according to claim 41.

46. The RNA molecule according to claim 40, the pharmaceutical composition according to claim 41, or the vaccine according to claim 45, for use in stimulating an immune response in a subject.

47. One or more 2′-O-methyl modified nucleotide triphosphate (NTP) in an RNA molecule, for use in enhancing the translation of the RNA molecule.
